(19) United States
(12) Patent Application Publication
(10) Pub. No.: US 2026/0015346 A1
(43) Pub. Date: Jan. 15, 2026

(54) GLUTATHIONE PEROXIDASE 4 (GPX4) INHIBITORS FOR THE TREATMENT OF CANCER

(71) Applicant: Ferro Therapeutics, Inc., Palo Alto, CA (US)

(72) Inventors: Chun Jiang, Hillsborough, CA (US); Rui Xu, Dallas, TX (US)

(21) Appl. No.: 18/994,575

(22) PCT Filed: Jul. 17, 2023
(86) PCT No.: PCT/US2023/027929

§ 371 Date: Jan. 14, 2025

Related U.S. Application Data
(60) Provisional application No. 63/389,584, filed on Jul. 15, 2022.

(51) Int. Cl. C07D 417/06; A61K 31/437; A61K 31/4725; C07D 471/04

(52) U.S. Cl. CPC C07D 417/06; A61K 31/437; A61K 31/4725; C07D 471/04

(57) ABSTRACT

The application relates to relates to glutathione peroxidase 4 (GPX4) inhibitors of the general Formulae I and II which induce ferroptosis in a cell and are useful for the treatment of cancer.

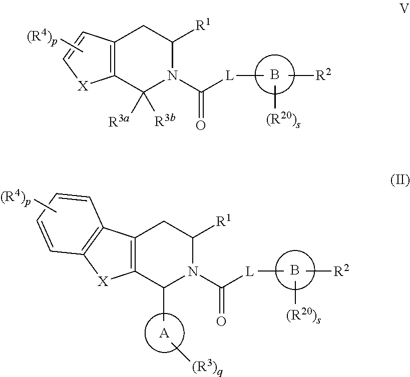

### CROSS-REFERENCE TO RELATED APPLICATIONS

This application claims the benefit under 35 U.S.C. § 119(e) of U.S. Provisional Application Ser. No. 63/389,584 filed Jul. 15, 2022, the contents of which are hereby incorporated by reference in its entirety.

### BACKGROUND

Glutathione peroxidase 4 (GPX4) can directly reduce phospholipid hydroperoxide. Depletion of GPX4 induces lipid peroxidation-dependent cell death. Cancer cells in a drug-induced, therapy-resistant state have an enhanced dependence on the lipid peroxidase activity of GPX4 to prevent undergoing ferroptotic cell death. Studies have shown that lipophilic antioxidants, such as Ferrostatin, can rescue cells from GPX4 inhibition-induced ferroptosis. For instance, mesenchymal state GPX4-knockout cells can survive in the presence of Ferrostatin, however, when the supply of Ferrostatin is terminated, these cells undergo ferroptosis (see, e.g., Viswanathan et al., Nature 547:453-7, 2017). It has also been experimentally determined that that GPX4i can be rescued by blocking other components of the ferroptosis pathways, such as lipid ROS scavengers (Ferrostatin, Liproxstatin), lipoxygenase inhibitors, iron chelators and caspase inhibitors, which an apoptotic inhibitor does not rescue. These findings are suggestive of non-apoptotic, iron-dependent, oxidative cell death (i.e., ferroptosis). Accordingly, a GPX4 inhibitor can be useful to induce ferroptotic cancer cell death and thus treat cancer.

### SUMMARY

The present disclosure relates to compounds having ferroptosis inducing activity, and methods of using the compounds for the treatment of cancer.

In an aspect, the present disclosure provides compositions comprising compounds according to Formula I or II:

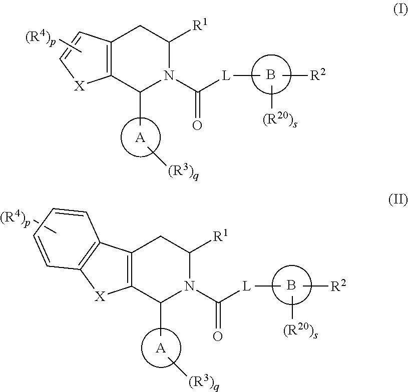

or a tautomer, stereoisomer, mixture of stereoisomers, isotopically enriched analog, or pharmaceutically acceptable salt thereof, wherein ring A, ring B, X, L, R¹, R², R³, R⁴, R²⁰, p, q, and s are as provided herein.

In some embodiments, a compound provided herein, or a tautomer, stereoisomer, mixture of stereoisomers, isotopically enriched analog, or pharmaceutically acceptable salt thereof, can modulate (e.g., inhibit) the activity of GPX4. In some embodiments, a compound provided herein, or a tautomer, stereoisomer, mixture of stereoisomers, isotopically enriched analog, or pharmaceutically acceptable salt thereof, can induce ferroptosis in a cell (e.g., a cancer cell). In some embodiments, a compound provided herein, or a tautomer, stereoisomer, mixture of stereoisomers, isotopically enriched analog, or pharmaceutically acceptable salt thereof, is useful in the treatment of a disease, disorder, or condition, such as a cancer.

In another aspect, the present disclosure provides pharmaceutical compositions comprising a compound provided herein, or a tautomer, stereoisomer, mixture of stereoisomers, isotopically enriched analog, or pharmaceutically acceptable salt thereof, and a pharmaceutically acceptable carrier.

In another aspect, the present disclosure provides a method of inhibiting GPX4 in a cell, comprising contacting a cell with an effective amount of a compound provided herein, or a tautomer, stereoisomer, mixture of stereoisomers, isotopically enriched analog, or pharmaceutically acceptable salt thereof. In some embodiments, the cell is located within a subject, such as a human. In some embodiments, the cell is a cancer cell.

In another aspect, the present disclosure provides a method of inducing ferroptosis in a cell, comprising contacting a cell with an effective amount of a compound provided herein, or a tautomer, stereoisomer, mixture of stereoisomers, isotopically enriched analog, or pharmaceutically acceptable salt thereof. In some embodiments, the cell is located within a subject, such as a human. In some embodiments, the cell is a cancer cell.

In a further aspect, the present disclosure provides a method of treating a disease, disorder, or condition in a subject in need thereof, comprising administering to the subject a therapeutically effective amount of a compound provided herein, or a tautomer, stereoisomer, mixture of stereoisomers, isotopically enriched analog, or pharmaceutically acceptable salt thereof. In some embodiments, the disease, disorder, or condition is a cancer. In some embodiments, the cancer is adrenocortical cancer, anal cancer, biliary cancer, bladder cancer, bone cancer, brain cancer, breast cancer, cervical cancer, colon cancer, endometrial cancer, esophageal cancer, head and neck cancer, intestinal cancer, liver cancer, lung cancer, oral cancer, ovarian cancer, pancreatic cancer, renal cancer, prostate cancer, salivary gland cancer, skin cancer, stomach cancer, testicular cancer, throat cancer, thyroid cancer, uterine cancer, vaginal cancer, sarcoma, or a soft tissue carcinoma. In some embodiments, the cancer is osteosarcoma, glioma, astrocytoma, neuroblastoma, cancer of the small intestine, bronchial cancer, small cell lung cancer, non-small cell lung cancer, basal cell carcinoma, or melanoma. In some embodiments, the cancer is a hematologic cancer. In some embodiments, the hematologic cancer is acute lymphoblastic leukemia (ALL), acute myeloid leukemia (AML), lymphoma (e.g., Hodgkin's lymphoma, Non-Hodgkin's lymphoma, Burkitt's lymphoma), chronic lymphocytic leukemia (CLL), chronic myelogenous leukemia (CML), Hairy Cell chronic myelogenous leukemia (CML), or multiple myeloma.

In some embodiments, a method provided herein further comprises administering a therapeutically effective amount of additional therapeutic agent. In some embodiments, the therapeutic agent is a platinating agent, an alkylating agent, an anti-cancer antibiotic, an antimetabolite, a topoisomerase I inhibitor, a topoisomerase II inhibitor, or an antimicrotubule agent.

### DETAILED DESCRIPTION

The present disclosure provides compounds (e.g., compounds of any one of Formulas I, I-a, IA, IB, IC, ID, IE, IF, IG, IH, IJ, IK, IL, IM, IN, IP, IQ, IR, II, IIA, IIB, IIC, IID, IIE, IIF, IIG, IIH, IIJ, IIK, IIL, IIM, IIN, or IlIP), which compounds may possess useful GPX4 inhibitory activity, and may be used in the treatment or prophylaxis of a disease, disorder, or condition in which GPX4 or ferroptosis plays a role. In particular, certain compounds provided herein may possess useful inhibitory activity of GPX4, and/or may be ferroptosis inducers or activators. The present disclosure also provides pharmaceutical compositions comprising one or more compounds provided herein together with a pharmaceutically acceptable carrier, as well as methods of making and using the compounds and compositions. The present disclosure also provides methods for inhibiting GPX4 and/or inducing or activating ferroptosis. In an aspect, the present disclosure provides a method for treating a disease, disorder, or condition in a subject in need of such treatment, which method comprises administering to the subject a therapeutically effective amount of a compound or composition provided herein. In some embodiments, the disease, disorder, or condition is ameliorated, treated, inhibited, or reduced by inhibition of GPX4 and/or inducement or activation of ferroptosis. Also provided herein is the use of certain compounds provided herein in the manufacture of a medicament for the treatment of a disease, disorder, or condition ameliorated, treated, inhibited, or reduced by inhibition of GPX4 and/or inducement or activation of ferroptosis. In some embodiments, the disease, disorder, or condition is ameliorated, treated, inhibited, or reduced by inhibition of GPX4 and/or inducement or activation of ferroptosis. In some embodiments, the disease, disorder, or condition is a cancer (e.g., as described herein).

As used in this specification and the appended claims, the singular forms “a,” “an” and “the” include plural referents unless the context clearly indicates otherwise. Thus, for example, reference to “a protein” includes more than one protein, and reference to “a compound” refers to more than one compound.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

It is to be understood that both the foregoing general description, including the drawings, and the following detailed description are exemplary and explanatory only and are not restrictive of this disclosure. The section headings used herein are for organizational purposes only and not to be construed as limiting the subject matter described.

### Definitions

In reference to the present disclosure, the technical and scientific terms used in the descriptions herein will have the meanings commonly understood by one of ordinary skill in the art, unless specifically defined otherwise. Accordingly, the following terms are intended to have the meanings as described below.

“Ferroptosis” refers to a form of cell death understood in the art as involving generation of reactive oxygen species mediated by iron, and characterized by, in part, lipid peroxidation.

“Ferroptosis inducer” or “ferroptosis activator” refers to an agent which induces, promotes or activates ferroptosis.

“GPX4 inhibitor” refers to any agent that inhibits the activity of the enzyme glutathione peroxidase 4 (GPX4). A GPX4 inhibitor can be either a direct or indirect inhibitor. GPX4 is a phospholipid hydroperoxidase that in catalyzing the reduction of hydrogen peroxide and organic peroxides, thereby protects cells against membrane lipid peroxidation, or oxidative stress. GPX4 has a selenocysteine in the active site that is oxidized to a selenenic acid by the peroxide to afford a lipid-alcohol. The glutathione acts to reduce the selenenic acid (—SeOH) back to the selenol (—SeH). Should this catalytic cycle be disrupted, cell death occurs through an intracellular iron-mediated process known as ferroptosis.

“Subject” as used herein refers to a mammal, for example a dog, a cat, a horse, or a rabbit. In certain embodiments, the subject is a non-human primate, for example a monkey, chimpanzee, or gorilla. In certain embodiments, the subject is a human, sometimes referred to herein as a patient.

“Treating” or “treatment” of a disease, disorder, or syndrome, as used herein, includes (i) preventing the disease, disorder, or syndrome from occurring in a subject, i.e. causing the clinical symptoms of the disease, disorder, or syndrome not to develop in an animal that may be exposed to or predisposed to the disease, disorder, or syndrome but does not yet experience or display symptoms of the disease, disorder, or syndrome; (ii) inhibiting the disease, disorder, or syndrome, i.e., arresting its development; and (iii) relieving the disease, disorder, or syndrome, i.e., causing regression of the disease, disorder, or syndrome. As is known in the art, adjustments for systemic versus localized delivery, age, body weight, general health, sex, diet, time of administration, drug interaction and the severity of the condition may be necessary, and will be ascertainable with routine experimentation by one of ordinary skill in the art, particularly in view of the guidance provided in the present disclosure.

“Therapeutically effective amount” refers to that amount which, when administered to an animal (e.g., human) for treating a disease, is sufficient to effect such treatment for the disease, disorder, or condition, or a symptom thereof. In certain embodiments, the treatment provides a therapeutic benefit such as amelioration of symptoms or slowing of disease progression. For example, a therapeutically effective amount may be an amount sufficient to decrease a symptom of a disease or condition of as described herein.

“Alkyl” refers to a straight or branched chain hydrocarbon group of 1 to 20 carbon atoms (C₁-C₂₀ or C_{1 20}), 1 to 12 carbon atoms (C₁-C₁₂ or C_{1 12}), or 1 to 8 carbon atoms (C₁-C₈ or C₁₈). Exemplary “alkyl” includes, but are not limited to, methyl, ethyl, n-propyl, i-propyl, n-butyl, s-butyl, t-butyl, n-pentyl, and s-pentyl, and the like.

“Alkenyl” refers to a straight or branched chain hydrocarbon group of 2 to 20 carbon atoms (C₂-C₂₀ or C₂₋₂₀), 2 to 12 carbon atoms (C₂-C₁₂ or C₂₋₁₂), or 2 to 8 carbon atoms (C₂-C₈ or C₂₋₈), having at least one double bond. Exemplary “alkenyl” includes, but are not limited to, vinyl, ethenyl, allyl, isopropenyl, 1-propenyl, 2-methyl-1-propenyl, 1-butenyl, 2-butenyl, 3-butenyl, 2-ethyl-1-butenyl, 3-methyl-2-butenyl, 1-pentenyl, 2-pentenyl, 3-pentenyl, 4-pentenyl, 4-methyl-3-pentenyl, 1-hexenyl, 2-hexenyl, 3-hexenyl, 4-hexenyl and 5-hexenyl, and the like.

“Alkynyl” refers to a straight or branched chain hydrocarbon group of 2 to 12 carbon atoms (C₂-C₁₂ or C₂-12), 2 to 8 carbon atoms (C₂-C₈ or C₂₋₈), containing at least one triple bond. Exemplary “alkynyl” includes ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl, and the like.

“Alkylene,” “alkenylene” and “alkynylene” refers to a straight or branched chain divalent hydrocarbon radical of the corresponding alkyl, alkenyl, and alkynyl, respectively. In certain embodiments, “alkyl,” “alkenyl,” and “alkynyl” can represent the corresponding “alkylene,” “alkenylene,” and “alkynylene,” such as, by way of example and not limitation, cycloalkylalkyl-, heterocycloalkylalkyl-, arylalkyl-, heteroarylalkyl-, cycloalkylalkenyl-, heterocycloalkylalkenyl-, arylalkenyl-, heteroarylalkenyl-, cycloalkylalkynyl-, heterocycloalkylalkynyl-, arylalkynyl-, heteroarylalkynyl-, and the like, wherein the cycloalkyl, heterocycloalkyl, aryl, and heteroaryl group is connected, as a substituent via the corresponding alkylene, alkenylene, or alkynylene group.

“Lower” in reference to substituents refers to a group having between one and six carbon atoms. For example, a lower alkyl group refers to an alkyl group including 1 to 6 carbon atoms (e.g., C₁₋₆alkyl), while a lower alkenyl group refers to an alkenyl group including 2 to 6 carbon atoms (e.g., C₂_alkenyl).

“Alkylhalo” or “haloalkyl” refers to a straight or branched chain hydrocarbon group of 1 to 20 carbon atoms (C₁-C₂₀ or C₁₋₂₀), 1 to 12 carbon atoms (C₁-C₁₂ or C₁₋₁₂), or 1 to 8 carbon atoms (C₁-C₈ or C₁₋₈) wherein one or more (e.g., one to three, or one) hydrogen atom is replaced by a halogen (e.g., Cl, F, etc.). In certain embodiments, the term “alkylhalo” refers to an alkyl group as defined herein, wherein one hydrogen atom is replaced by a halogen (e.g., Cl, F, etc.). In certain embodiments, the term “alkylhalo” refers to an alkylchloride.

“Alkenylhalo” or “haloalkenyl” refers to a straight or branched chain hydrocarbon group of 2 to 20 carbon atoms (C₂-C₂₀ or C₂₋₂₀), 2 to 12 carbon atoms (C₂-C₁₂ or C₂₋₁₂), or 2 to 8 carbon atoms (C₂-C₈ or C₂₋₈), having at least one double bond, wherein one or more (e.g., one to three, or one) hydrogen atom is replaced by a halogen (e.g., Cl, F, etc.). In certain embodiments, the term “alkenylhalo” refers to an alkenyl group as defined herein, wherein one hydrogen atom is replaced by a halogen (e.g., Cl, F, etc.). In certain embodiments, the term “alkenylhalo” refers to an alkenylchloride.

“Heteroalkyl” refers to a straight or branched chain hydrocarbon group of 1 to 20 carbon atoms (C₁-C₂₉ or C₁₋₂₀), 1 to 12 carbon atoms (C₁-C₁₂ or C₁₋₁₂), or 1 to 8 carbon atoms (C₁-C₈ or C₁₋₈), wherein 1 to 3 carbon atoms are replaced by a heteroatom. Heteroatoms and/or heteroatomic groups which can replace the carbon atoms include, but are not limited to, —O—, —S—, —NR⁴⁰—, —PH—, —C(O)—, —S(O)—, —S(O)₂—, —S(O)NR⁴⁰—, —S(O)₂NR⁴⁰—, and the like, including combinations thereof, wherein each R⁴⁰ is independently hydrogen or lower alkyl unless otherwise specified.

“Cycloalkyl” refers to any stable monocyclic or polycyclic system which consists of carbon atoms, any ring of which being saturated. “Cycloalkenyl” refers to any stable monocyclic or polycyclic system which consists of carbon atoms, with at least one ring thereof being partially unsaturated. Examples of cycloalkyls include, but are not limited to, cyclopropyl, cyclobutyl, cyclopentyl, cyclohexyl, cycloheptyl, cyclooctyl, bicycloalkyls and tricycloalkyls (e.g., adamantyl).

“Heterocycloalkyl” or “heterocyclyl” refers to a 4 to 14 membered, mono- or polycyclic (e.g., bicyclic), non-aromatic hydrocarbon ring, wherein 1 to 3 carbon atoms are replaced by a heteroatom. Heteroatoms and/or heteroatomic groups which can replace the carbon atoms include, but are not limited to, —O—, —S—, —S—O—, —NR⁴⁰—, —PH—, —C(O)—, —S(O)—, —S(O)₂—, —S(O)NR⁴⁰—, —S(O)₂NR⁴⁰—, and the like, including combinations thereof, where each R⁴⁰ is independently hydrogen or lower alkyl. Examples include thiazolidinyl, thiadiazolyl, triazinyl, morpholinyl, pyrrolidinonyl, pyrrolidinyl, piperidinyl, piperazinyl, 2,3-dihydrofuranyl, dihydropyranyl, hydantoinyl, valerolactamyl, oxiranyl, oxetanyl, tetrahydrofuranyl, tetrahydropyranyl, dihydropyridinyl, tetrahydropyridinyl, tetrahydropyrimidinyl, tetrahydrothiophenyl, tetrahydrothiopyranyl, and the like. In certain embodiments, the “heterocycloalkyl” or “heterocyclyl” is a substituted or unsubstituted 4 to 7 membered monocyclic ring, wherein 1 to 3 carbon atoms are replaced by a heteroatom as described above.

In certain embodiments, the “heterocycloalkyl” or “heterocyclyl” is a 4 to 10, or 4 to 9, or 5 to 9, or 5 to 7, or 5 to 6 membered mono- or polycyclic (e.g., bicyclic) ring, wherein 1 to 3 carbon atoms are replaced by a heteroatom as described above. In certain embodiments, when the “heterocycloalkyl” or “heterocyclyl” is a substituted or unsubstituted bicyclic ring, one ring may be aromatic, provided at least one ring is non-aromatic, regardless of the point of attachment to the remainder of the molecule (e.g., indolinyl, isoindolinyl, and the like).

“Aryl” refers to a 6 to 14-membered, mono- or bi-carbocyclic ring, wherein the monocyclic ring is aromatic and at least one of the rings in the bicyclic ring is aromatic. Unless stated otherwise, the valency of the group may be located on any atom of any ring within the radical, valency rules permitting. Examples of “aryl” groups include phenyl, naphthyl, indenyl, biphenyl, phenanthrenyl, naphthacenyl, and the like.

“Heteroaryl” means an aromatic heterocyclic ring, including monocyclic and polycyclic (e.g., bicyclic) ring systems, where at least one carbon atom of one or both of the rings is replaced with a heteroatom independently selected from nitrogen, oxygen, and sulfur, or at least two carbon atoms of one or both of the rings are replaced with a heteroatom independently selected from nitrogen, oxygen, and sulfur. In certain embodiments, the heteroaryl can be a 5 to 6 membered monocyclic, or 7 to 11 membered bicyclic ring systems. Examples of “heteroaryl” groups include pyrrolyl, pyrazolyl, imidazolyl, pyrazinyl, oxazolyl, isoxazolyl, thiazolyl, furyl, thienyl, pyridyl, pyrimidyl, benzothiazolyl, purinyl, benzimidazolyl, indolyl, isoquinolyl, quinoxalinyl, quinolyl, and the like.

“Bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In certain embodiments, a bridged bicyclic group has 5-12 ring members and 0-4 heteroatoms independently selected from nitrogen, oxygen, and sulfur. Such bridged bicyclic groups include those groups set forth below where each group is attached to the rest of the molecule at any substitutable carbon or nitrogen atom. Exemplary bridged bicyclics include, but are not limited to:

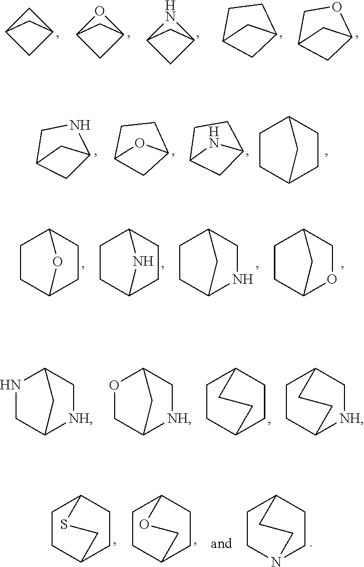

“Fused ring” refers a ring system with two or more rings having at least one bond and two atoms in common. A “fused aryl” and a “fused heteroaryl” refer to ring systems having at least one aryl and heteroaryl, respectively, that share at least one bond and two atoms in common with another ring.

“Halogen” or “halo” refers to fluorine, chlorine, bromine and iodine.

“Acyl” refers to —C(O)R⁴³, where R⁴³ is hydrogen, or an optionally substituted alkyl, heteroalkyl, cycloalkyl, cycloalkylalkyl, heterocycloalkyl, heterocycloalkylalkyl, aryl, arylalkyl, heteroaryl, or heteroarylalkyl as defined herein. Exemplary acyl groups include, but are not limited to, formyl, acetyl, cyclohexylcarbonyl, cyclohexylmethylcarbonyl, benzoyl, benzylcarbonyl, and the like.

“Alkyloxy” or “alkoxy” refers to —OR⁴⁴, wherein R⁴⁴ is an optionally substituted alkyl.

“Aryloxy” refers to —OR⁴⁵, wherein R⁴⁵ is an optionally substituted aryl.

“Carboxy” refers to —COO or COOM, wherein M is H or a counterion (e.g., a cation, such as Na⁺, Ca²⁺, Mg²⁺, etc.).

“Carbamoyl” refers to —C(O)NR⁴⁶R⁴⁶, wherein each R⁴⁶ is independently selected from H or an optionally substituted alkyl, cycloalkyl, cycloalkylalkyl, heterocycloalkyl, heterocylcoalkylalkyl, aryl, arylalkyl, heteroaryl, or heteroarylalkyl.

“Ester” refers to a group such as —C(═O)OR⁴⁷, alternatively illustrated as —C(O)OR⁴⁷, wherein R⁴⁷ is selected from an optionally substituted alkyl, cycloalkyl, cycloalkylalkyl, heterocycloalkyl, heterocyclolalkylalkyl, aryl, arylalkyl, heteroaryl, and heteroarylalkyl.

“Ether” refers to the group -alkyl-O-alkyl, where the term alkyl is as defined herein.

“Sulfanyl” refers to —SR⁴⁸, wherein R⁴⁸ is selected from an optionally substituted alkyl, cycloalkyl, cycloalkylalkyl, heterocycloalkyl, heterocycloalkylalkyl, aryl, arylalkyl, heteroaryl, and heteroarylalkyl. For example, —SR⁴⁸, wherein R⁴⁸ is an alkyl is an alkylsulfanyl.

“Sulfonyl” refers to —S(O)₂—, which may have various substituents to form different sulfonyl groups including sulfonic acids, sulfonamides, sulfonate esters, and sulfones. For example, —S(O)₂R⁴⁹, wherein R⁴⁹ is an alkyl refers to an alkylsulfonyl. In certain embodiments of —S(O)₂R⁴⁹, R⁴⁹ is selected from an optionally substituted alkyl, cycloalkyl, cycloalkylalkyl, heterocycloalkyl, heterocycloalkylalkyl, aryl, arylalkyl, heteroaryl, and heteroarylalkyl.

“Sulfinyl” refers to —S(O)—, which may have various substituents to form different sulfinyl groups including sulfinic acids, sulfinamides, and sulfinyl esters. For example, —S(O)R⁵⁰, wherein R⁵⁰ is an alkyl refers to an alkylsulfinyl. In certain embodiments of —S(O)R⁵⁰, R⁵⁰ is selected from an optionally substituted alkyl, cycloalkyl, cycloalkylalkyl, heterocycloalkyl, heterocycloalkylalkyl, aryl, arylalkyl, heteroaryl, and heteroarylalkyl.

“Silyl” refers to Si, which may have various substituents, for example —SiR⁵¹R⁵¹R⁵¹, where each R⁵¹ is independently selected from alkyl, cycloalkyl, cycloalkylalkyl, heterocycloalkyl, heterocycloalkylalkyl, aryl, arylalkyl, heteroaryl, and heteroarylalkyl. As defined herein, any heterocycloalkyl or heteroaryl group present in a silyl group has from 1 to 3 heteroatoms selected independently from O, N, and S.

“Amino” or “amine” refers to the group —NR⁵²R⁵² or —N⁺R⁵²R⁵²R⁵², wherein each R⁵² is independently selected from hydrogen and an optionally substituted alkyl, cycloalkyl, heterocycloalkyl, alkyloxy, aryl, heteroaryl, heteroarylalkyl, acyl, —C(O)—O-alkyl, sulfanyl, sulfinyl, sulfonyl, and the like. Exemplary amino groups include, but are not limited to, dimethylamino, diethylamino, trimethylammonium, triethylammonium, methylysulfonylamino, furanyl-oxy-sulfamino, and the like.

“Amide” refers to a group such as —C(═O)NR⁵³R⁵³, wherein each R⁵³ is independently selected from H and an optionally substituted alkyl, cycloalkyl, cycloalkylalkyl, heterocycloalkyl, heterocycloalkylalkyl, aryl, arylalkyl, heteroaryl, and heteroarylalkyl.

“Carbamate” refers to a group such as —O—C(═O)NR⁵³R⁵³ or —NR⁵³—C(═O)OR⁵³, wherein each R⁵³ is independently selected from H and an optionally substituted alkyl, cycloalkyl, cycloalkylalkyl, heterocycloalkyl, heterocycloalkylalkyl, aryl, arylalkyl, heteroaryl, and heteroarylalkyl.

“Sulfonamide” refers to —S(O)₂NR⁵⁴R⁵⁴, wherein each R⁵⁴ is independently selected from H and an optionally substituted alkyl, heteroalkyl, heteroaryl, heterocycle, alkenyl, alkynyl, arylalkyl, heteroarylalkyl, heterocyclylalkyl, alkylene-C(O)—OR⁵⁵, or alkylene-O—C(O)—OR⁵⁵, where R⁵⁵ is selected from H, alkyl, heteroalkyl, cycloalkyl, heterocyclyl, aryl, heteroaryl, alkenyl, alkynyl, arylalkyl, heterocycloalkyl, heteroarylalkyl, amino, and sulfinyl.

“Adamantyl” refers to tricyclo[3.3.1.1^{3,7}]decanyl, where bonding can be via a 3-coordinated carbon site or a 2-coordinated carbon site (i.e., 1-adamantyl or 2-adamantyl). In certain embodiments, “adamantyl” refers to a compound of structural formula:

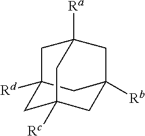

where optional substitutions can be present on one or more of R^{a}, R^{b}, R^{c}, and R^{d}. Adamantyl includes substituted adamantyl, e.g., 1- or 2-adamantyl, substituted by one or more substituents, including alkyl, halo, —OH, —NH₂, and alkoxy. Exemplary derivatives include methyladamatane, haloadamantane, hydroxyadamantane, and aminoadamantane (e.g., amantadine).

“N-protecting group” as used herein refers to those groups intended to protect a nitrogen atom against undesirable reactions during synthetic procedures. Exemplary N-protecting groups include, but is not limited to, acyl groups such acetyl and t-butylacetyl, pivaloyl, alkoxycarbonyl groups such as methyloxycarbonyl and t-butyloxycarbonyl (Boc), aryloxycarbonyl groups such as benzyloxycarbonyl (Cbz) and fluorenylmethoxycarbonyl (Fmoc) and aroyl groups such as benzoyl. N-protecting groups are described in Greene's Protective Groups in Organic Synthesis, 5th Edition, P. G. M. Wuts, ed., Wiley (2014).

“Optional” or “optionally” refers to a described event or circumstance may or may not occur, and that the description includes instances where the event or circumstance occurs and instances where the event or circumstance does not. For example, “optionally substituted alkyl” refers to an alkyl group that may or may not be substituted and that the description encompasses both substituted alkyl group and unsubstituted alkyl group.

“Substituted” as used herein means one or more hydrogen atoms of the group is replaced with a substituent atom or group commonly used in pharmaceutical chemistry. Each substituent can be the same or different. Examples of suitable substituents include, but are not limited to, alkyl, alkenyl, alkynyl, cycloalkyl, aryl, arylalkyl, heterocycloalkyl, heteroaryl, —OR⁵⁶ (e.g., hydroxyl, alkyloxy (e.g., methoxy, ethoxy, and propoxy), ether, ester, carbamate, etc.), hydroxyalkyl, —C(O)O-alkyl, —O-alkyl-O-alkyl, haloalkyl, alkyl-O-alkyl, SR⁵⁶ (e.g., —SH, —S-alkyl, —S-aryl, —S-heteroaryl, arylalkyl-S—, etc.), S⁺R⁵⁶₂, S(O)R⁵⁶, SO₂R⁵⁶, NR⁵⁶R⁵⁷ (e.g., primary amine (i.e., NH₂), secondary amine, tertiary amine, amide, carbamate, urea, etc.), hydrazide, halo, nitrile, nitro, sulfide, sulfoxide, sulfone, sulfonamide, —SH, carboxy, aldehyde, keto, carboxylic acid, ester, amide, imine, and imide (e.g., —C(O)NR⁵⁶C(O)R⁵⁷), including seleno and thio derivatives thereof, wherein each R⁵⁶ and R⁵⁷ are independently alkyl, alkenyl, alkynyl, heteroalkyl, cycloalkyl, cycloalkylalkyl, heterocycloalkyl, heterocycloalkylalkyl, aryl, arylalkyl, heteroaryl, or heteroarylalkyl, and wherein each of the substituents can be optionally further substituted. In embodiments in which a functional group with an aromatic carbon ring is substituted, such substitutions will typically number less than about 10 substitutions, or about 1 to 5, with about 1 or 2 substitutions in certain embodiments.

“Pharmaceutically acceptable salt” is meant to include salts of the active compounds which are prepared with relatively nontoxic acids or bases, depending on the particular substituents found on the compounds described herein. When compounds as disclosed herein contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds as disclosed herein contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, phosphoric, partially neutralized phosphoric acids, sulfuric, partially neutralized sulfuric, hydroiodic, or phosphorous acids and the like, as well as the salts derived from relatively nontoxic organic acids like acetic, propionic, isobutyric, maleic, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present disclosure may contain both basic and acidic functionalities that allow the compounds to be converted into either base or acid addition salts. Lists of suitable salts are found in Remington's Pharmaceutical Sciences, 17th Ed., Mack Publishing Company, Easton, Pa., (1985) and Journal of Pharmaceutical Science, 66:2 (1977), each of which is incorporated herein by reference in its entirety.

“Pharmaceutically acceptable carrier” or “pharmaceutically acceptable excipient” refers to an excipient, carrier or adjuvant that can be administered to a subject, together with at least one compound, and which does not destroy the pharmacological activity thereof and is generally safe, nontoxic and neither biologically nor otherwise undesirable when administered in doses sufficient to deliver a therapeutic amount of the agent.

Any compound or structure given herein, is also intended to represent unlabeled forms as well as isotopically labeled forms of the compounds. These forms of compounds may also be referred to as “isotopically enriched analogs.” Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Various isotopically labeled compounds of the present disclosure, for example those into which radioactive isotopes such as ³H, and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The term “isotopically enriched analogs” includes “deuterated analogs” of compounds described herein in which one or more hydrogens is/are replaced by deuterium, such as a hydrogen on a carbon atom. Such compounds exhibit increased resistance to metabolism and are thus useful for increasing the half-life of any compound when administered to a mammal, e.g., a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F, ³H, ¹¹C labeled compound may be useful for PET or SPECT or other imaging studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in a compound described herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen,” the position is understood to have hydrogen at its natural abundance isotopic composition. Accordingly, in the compounds of this disclosure any atom specifically designated as a deuterium (D) is meant to represent deuterium.

Some of the compounds exist as tautomers. Tautomers are in equilibrium with one another. For example, amide containing compounds may exist in equilibrium with imidic acid tautomers. Regardless of which tautomer is shown and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

The compounds as disclosed herein, or their pharmaceutically acceptable salts include an asymmetric center and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers.

A “stereoisomer” refers to a compound made up of the same atoms bonded by the same bonds but having different three-dimensional structures, which are not interchangeable. The present disclosure contemplates various stereoisomers and mixtures thereof and includes “enantiomers,” which refers to two stereoisomers whose molecules are non-superimposable mirror images of one another.

“Diastereomers” are stereoisomers that have at least two asymmetric atoms, but which are not mirror-images of each other.

Relative centers of the compounds as depicted herein are indicated graphically using the “thick bond” style (bold or parallel lines) and absolute stereochemistry is depicted using wedge bonds (bold or parallel lines).

### Compounds

In an aspect, the present disclosure provides a compound of Formula I:

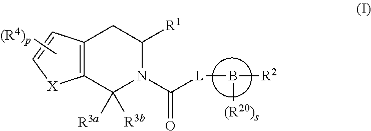

or a tautomer, stereoisomer, mixture of stereoisomers, isotopically enriched analog, or pharmaceutically acceptable salt thereof, wherein:
ring B is a heteroaryl;
L is absent or —NR²¹—;
X is —O—, —S—, —NR⁹—, —CR⁵═CR⁵—, or —CR⁵═N—;
p is 0, 1, or 2;
s is 0, 1, 2, or 3;
R¹ is C₁-C₆alkyl, C₂-C₆alkenyl, C₂-C₆alkynyl, C₁-C₆haloalkyl, C₃-C₁₀cycloalkyl, heteroaryl, —CN, —OR⁷, —C(O)OR⁶, —C(O)N(R⁷)₂, —OC(O)R⁶, —S(O)₂R⁸, —S(O)₂N(R⁷)₂, —S(O)N(R⁷)₂, —S(O)R⁸, —N(R⁷)₂, —NO₂, —C₁-C₆alkyl-OR⁷, or —Si(R¹⁵)₃; wherein the heteroaryl is unsubstituted or substituted with C₁-C₆alkyl;
R² is C₂alkynyl that is unsubstituted or substituted with C₁-C₆alkyl, C₂-C₆alkenyl, C₂-C₆alkynyl, C₃-C₁₀cycloalkyl, C₆-C₁₄aryl, C₇-C₁₅aralkyl, heteroaryl, or heterocyclyl;
R^{3a} is hydrogen and R^{3b} is

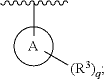

wherein q is 0, 1, 2, or 3; and ring A is C₄-C₁₀cycloalkyl, heterocyclyl, aryl, or heteroaryl; or
R^{3a} and R^{3b} together with the carbon atom to which they are attached form a C₄-C₁₀cycloalkyl or heterocyclyl; wherein the C₄-C₁₀cycloalkyl or heterocyclyl is unsubstituted or substituted with one to three R³;
each R³ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)N(R⁷)₂, —OC(O)R⁸, —C(O)R⁶, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R³ is independently unsubstituted or substituted with one to three R¹⁰;
each R⁴ is independently halo, —CN, —OH, —OR⁸, —NH₂, —NHR⁸, —N(R′)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R⁴ is independently unsubstituted or substituted with one to three R¹⁰;
each R⁵ is independently hydrogen, halo, —CN, —OH, —OR⁸, —NH₂, —NHR⁸, —N(R′)₂, —S(O)₂R⁸, —S(O)R⁸, —S(O)₂N(R⁷)₂, —S(O)N(R⁷)₂, —NO₂, —Si(R¹)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, or —C₂-C₆alkenylheteroaryl of R⁵ is independently unsubstituted or substituted with one to three R¹⁰;
each R⁶ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, or —C₂-C₆alkenylheteroaryl; wherein each R⁶ is independently unsubstituted or substituted with one to three R¹¹;
each R⁷ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₆cycloalkyl, —C₂-C₆alkenylC₃-C₆cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, —C₂-C₆alkenylheteroaryl, or two R⁷ together with the nitrogen atom to which they are attached, form a 4 to 7 membered heterocyclyl; wherein each R⁷ or ring formed thereby is independently unsubstituted or substituted with one to three R¹¹;
each R⁸ is independently C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, or —C₂-C₆alkenylheteroaryl; wherein each R⁸ is independently unsubstituted or substituted with one to three R¹¹;
R⁹ is hydrogen or C₁-C₆alkyl;
each R¹⁰ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR^{D}, —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, —P(O)(OR¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl, wherein each C₁-C₆alkyl, C₁-C₆haloalkyl, C₂-C₆alkenyl, C₂-C₆alkynyl, C₃-C₁₀cycloalkyl, heterocyclyl, aryl, or heteroaryl of R¹⁰ is independently unsubstituted or substituted with one to three R¹¹;
each R¹¹ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl;
each R¹² is independently hydrogen, C₁-C₆alkyl or C₃-C₁₀cycloalkyl;
each R¹³ is independently C₁-C₆alkyl or C₃-C₁₀cycloalkyl;
each R¹⁵ is independently C₁-C₆alkyl, C₂-C₆alkenyl, aryl, heteroaryl, —C₁-C₆alkylaryl, —C₂-C₆alkenylaryl, —C₁-C₆alkylheteroaryl, or —C₂-C₆alkenylheteroaryl;
each R²⁰ is independently halogen, C₁-C₆alkyl, or C₂-C₆alkenyl; and
R²¹ is hydrogen or C₁-C₆alkyl.

In some embodiments, when R^{3a} is hydrogen, and R^{3b} is

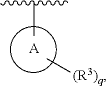

then at least one of the following is true:
1) R¹ is other than —C(O)OCH₃;
2) R² is —C₂alkynyl that is unsubstituted or substituted with one —CH₃; or
3) when R¹ is —C(O)OCH₃, R² is —CH₂Cl, then the moiety

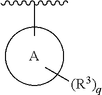

is other than 1,3-benzodioxol-5-yl, 4-nitrophenyl, 4-bromophenyl, cyclohexyl, furyl, or 4-methoxyphenyl.

In some embodiments, R^{3a} and R^{3b} together with the carbon atom to which they are attached form a C₄-C₁₀cycloalkyl or heterocyclyl; wherein the C₄-C₁₀cycloalkyl or heterocyclyl is unsubstituted or substituted with one to three R³. In some embodiments, R^{3a} and R^{3b} together with the carbon atom to which they are attached form a C₄-C₁₀cycloalkyl; wherein the C₄-C₁₀cycloalkyl is unsubstituted or substituted with one to three R³. In some embodiments, R^{3a} and R^{3b} together with the carbon atom to which they are attached form a heterocyclyl; wherein the heterocyclyl is unsubstituted or substituted with one to three R³. In some embodiments, R^{3a} and R^{3b} together with the carbon atom to which they are attached form a 4-6 membered heterocyclyl; wherein the 4-6 membered heterocyclyl is unsubstituted or substituted with one to three R³. In some embodiments, R^{3a} and R^{3b} together with the carbon atom to which they are attached form an azetidine or piperidine; wherein the azetidine or piperidine is unsubstituted or substituted with one to three R³. In some embodiments, R^{3a} and R^{3b} together with the carbon atom to which they are attached form an azetidine or piperidine; wherein the azetidine or piperidine is unsubstituted or substituted with methyl.

In an aspect, the present disclosure provides a compound of Formula I-a:

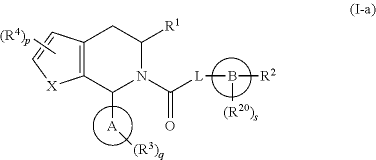

or a tautomer, stereoisomer, mixture of stereoisomers, isotopically enriched analog, or pharmaceutically acceptable salt thereof, wherein:
ring A is C₄-C₁₀cycloalkyl, heterocyclyl, aryl, or heteroaryl;
ring B is a heteroaryl;
L is absent or —NR²¹—;
X is —O—, —S—, —NR⁹—, —CR⁵═CR⁵—, or —CR⁵═N—;
p is 0, 1, or 2;
q is 0, 1, 2, or 3;
s is 0, 1, 2, or 3;
R¹ is C₁-C₆alkyl, C₂-C₆alkenyl, C₂-C₆alkynyl, C₁-C₆haloalkyl, C₃-C₁₀cycloalkyl, —CN, —OR⁷, —C(O)OR⁶, —C(O)N(R⁷)₂, —OC(O)R⁶, —S(O)₂R⁸, —S(O)₂N(R⁷)₂, —S(O)N(R⁷)₂, —S(O)R⁸, —N(R⁷)₂, —NO₂, —C₁-C₆alkyl-OR⁷, or —Si(R⁵)₃;
R² is C₂alkynyl that is unsubstituted or substituted with C₁-C₆alkyl, C₂-C₆alkenyl, C₂-C₆alkynyl, C₃-C₁₀cycloalkyl, C₆-C₁₄aryl, C₇-C₁₅aralkyl, heteroaryl, or heterocyclyl;
each R³ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)N(R⁷)₂, —OC(O)R⁸, —C(O)R⁶, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R³ is independently unsubstituted or substituted with one to three R¹⁰;
each R⁴ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R⁴ is independently unsubstituted or substituted with one to three R¹⁰;
each R⁵ is independently hydrogen, halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, or —C₂-C₆alkenylheteroaryl of R⁵ is independently unsubstituted or substituted with one to three R¹⁰;
each R⁶ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, or —C₂-C₆alkenylheteroaryl; wherein each R⁶ is independently unsubstituted or substituted with one to three R¹¹;
each R⁷ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₆cycloalkyl, —C₂-C₆alkenylC₃-C₆cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, —C₂-C₆alkenylheteroaryl, or two R⁷ together with the nitrogen atom to which they are attached, form a 4 to 7 membered heterocyclyl; wherein each R⁷ or ring formed thereby is independently unsubstituted or substituted with one to three R¹¹;
each R⁸ is independently C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, or —C₂-C₆alkenylheteroaryl; wherein each R⁸ is independently unsubstituted or substituted with one to three R¹¹;
R⁹ is hydrogen or C₁-C₆alkyl;
each R¹⁰ is independently halo, —CN, —OR¹², —NO₂, —N(R¹²)₂, —S(O)R¹³, —S(O)₂R¹³, —S(O)N(R¹²), —S(O)₂N(R¹²)₂, —Si(R¹²)₃, —C(O)R¹², —C(O)OR¹², —C(O)N(R¹²)₂, —NR²C(O)R¹², —OC(O)R¹², —OC(O)OR¹², —OC(O)N(R¹²)₂, —NR¹²C(O)OR¹², —OC(O)CHR¹²N(R¹²)₂, —P(O)(OR¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl, wherein each C₁-C₆alkyl, C₁-C₆haloalkyl, C₂-C₆alkenyl, C₂-C₆alkynyl, C₃-C₁₀cycloalkyl, heterocyclyl, aryl, or heteroaryl of R¹⁰ is independently unsubstituted or substituted with one to three R¹¹;
each R¹¹ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl;
each R¹² is independently hydrogen, C₁-C₆alkyl or C₃-C₁₀cycloalkyl;
each R¹³ is independently C₁-C₆alkyl or C₃-C₁₀cycloalkyl;
each R¹⁵ is independently C₁-C₆alkyl, C₂-C₆alkenyl, aryl, heteroaryl, —C₁-C₆alkylaryl, —C₂-C₆alkenylaryl, —C₁-C₆alkylheteroaryl, or —C₂-C₆alkenylheteroaryl;
each R²⁰ is independently halogen, C₁-C₆alkyl, or C₂-C₆alkenyl; and
R²¹ is hydrogen or C₁-C₆alkyl.

In some embodiments, at least one of the following is true:
1) R¹ is other than —C(O)OCH₃;
2) R² is —C₂alkynyl that is unsubstituted or substituted with one —CH₃; or
3) when R¹ is —C(O)OCH₃ and R² is —CH₂Cl, then the moiety

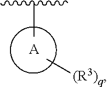

is other than 1,3-benzodioxol-5-yl, 4-nitrophenyl, 4-bromophenyl, cyclohexyl, furyl, or 4-methoxyphenyl.

In some embodiments, R¹ is other than —C(O)OR⁶ or R² is —C₂alkynyl that is unsubstituted or substituted with one —CH₃. In some embodiments, R¹ is other than —C(O)OCH₃ or R² is —C₂alkynyl that is unsubstituted or substituted with one —CH₃. In some embodiments, R¹ is other than —C(O)OR⁶ and R² is —C₂alkynyl that is unsubstituted or substituted with one —CH₃. In some embodiments, R¹ is other than —C(O)OCH₃ and R² is —C₂alkynyl that is unsubstituted or substituted with one —CH₃. In some embodiments, R¹ is other than —C(O)OR⁶. In some embodiments, R¹ is other than —C(O)OCH₃. In some embodiments, R² is —C₂alkynyl that is unsubstituted or substituted with one —CH₃. In some embodiments, R² is —C₂alkynyl.

In some embodiments, the compound is a compound according to Formula IA:

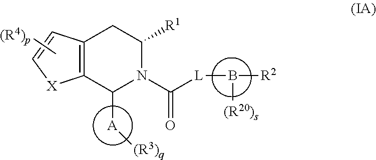

or a tautomer, stereoisomer, mixture of stereoisomers, isotopically enriched analog, or pharmaceutically acceptable salt thereof, wherein each of ring A, ring B, X, R¹, R², R³, R⁴, R²⁰, p, q, and s are independently as defined herein.

In some embodiments, the compound is a compound according to Formula IB:

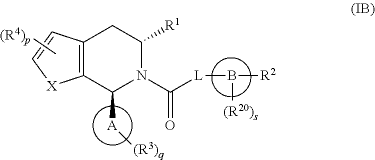

or a tautomer, stereoisomer, mixture of stereoisomers, isotopically enriched analog, or pharmaceutically acceptable salt thereof, wherein each of ring A, ring B, X, R¹, R², R³, R⁴, R²⁰, p, q, and s are independently as defined herein.

In some embodiments, for a compound of Formula I, I-a, IA, or IB, ring B includes a single heteroatom. In some embodiments, ring B includes at least one heteroatom, such as 1, 2, or 3 heteroatoms. In some embodiments, ring B includes 2 heteroatoms. In some embodiments, ring B includes one or more heteroatoms selected from S. O, and N. In some embodiments, ring B includes a heteroatom that is N. In some embodiments, ring B includes a heteroatom that is S. In some embodiments, ring B includes heteroatoms that are N and S. In some embodiments, ring B is a bicyclic heteroaryl. In some embodiments, ring B is a monocyclic heteroaryl. In some embodiments, ring B is a 5-10 membered heteroaryl. In some embodiments, ring B is a 5-6 membered heteroaryl. In some embodiments, ring B is a 5-6 membered heteroaryl including at least one heteroatom selected from S, O, and N. In some embodiments, ring B has the structure:

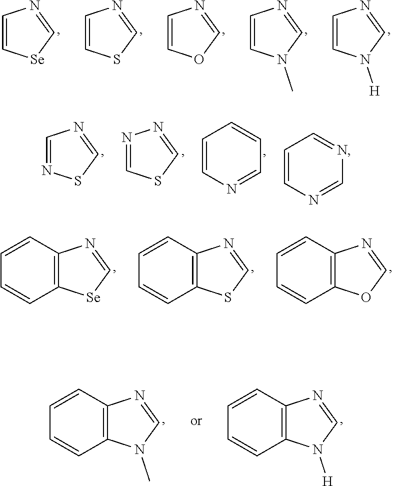

which structure is substituted with R² and s R²⁰s. In some embodiments, ring B has a structure shown above and is substituted with R² and 0 R²⁰s (e.g., s is 0). In some embodiments, ring B has the structure

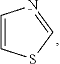

which structure is substituted with R² and s R²⁰s.

In some embodiments, the compound is a compound of Formula IC:

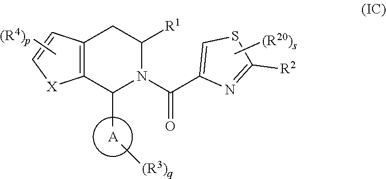

or a tautomer, stereoisomer, mixture of stereoisomers, isotopically enriched analog, or pharmaceutically acceptable salt thereof, wherein each of ring A, X, R¹, R², R³, R⁴, R²⁰, p, q, and s are independently as defined herein.

In some embodiments, the compound is a compound of Formula ID:

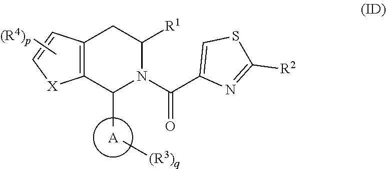

or a tautomer, stereoisomer, mixture of stereoisomers, isotopically enriched analog, or pharmaceutically acceptable salt thereof, wherein each of ring A, X, R¹, R², R³, R⁴, p, and q are independently as defined herein.

In some embodiments, the compound is a compound of Formula IE:

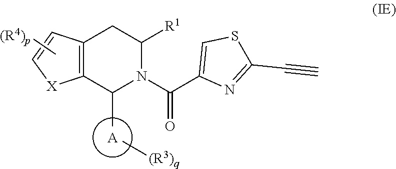

or a tautomer, stereoisomer, mixture of stereoisomers, isotopically enriched analog, or pharmaceutically acceptable salt thereof, wherein each of ring A, X, R¹, R³, R⁴, p, and q are independently as defined herein.

In some embodiments, the compound is a compound of Formula IF:

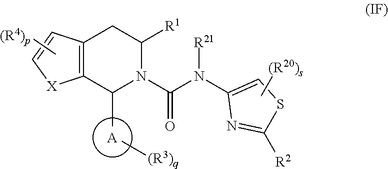

or a tautomer, stereoisomer, mixture of stereoisomers, isotopically enriched analog, or pharmaceutically acceptable salt thereof, wherein each of ring A, X, R¹, R², R³, R⁴, R²⁰, R²¹, p, q, and s are independently as defined herein.

In some embodiments, the compound is a compound of Formula IG:

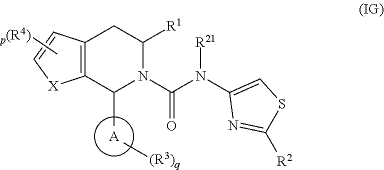

or a tautomer, stereoisomer, mixture of stereoisomers, isotopically enriched analog, or pharmaceutically acceptable salt thereof, wherein each of ring A, X, R¹, R², R³, R⁴, R²¹, p, and q are independently as defined herein.

In some embodiments, the compound is a compound of Formula TH:

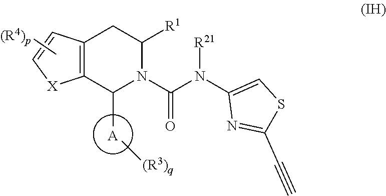

or a tautomer, stereoisomer, mixture of stereoisomers, isotopically enriched analog, or pharmaceutically acceptable salt thereof, wherein each of ring A, X, R¹, R², R³, R⁴, R²¹, p, and q are independently as defined herein.

In some embodiments, for a compound of Formula IF, IG, or IH, R²¹ is H. In some embodiments, R²¹ is C₁-C₆alkyl.

In some embodiments, for a compound of any one of Formulas I, I-a, IA, IB, IC, ID, IE, IF, IG, or IH:
ring A is aryl or heteroaryl;
X is —O—, —S—, —NH—, —CH═CH—, or —CH═N—;
p is 0, 1 or 2;
q is 1;
R¹ is C₁-C₆alkyl, —C(O)O—C₁-C₆alkyl, or —C(O)N(C₁-C₆alkyl)₂;
R³ is halo, —NHR⁸, —S(O)₂N(R⁷)₂, —C(O)OR⁶, —C(O)N(R⁷)₂, or heterocyclyl, wherein the heterocyclyl is independently unsubstituted or substituted with one to three R¹⁰;
each R⁴ is independently —OR⁸;
R⁶ is C₁-C₆alkyl;
each R⁷ is independently hydrogen, C₁-C₆alkyl, or C₃-C₁₀cycloalkyl, wherein each R⁷ is independently unsubstituted or substituted with one to three R¹¹;
each R⁸ is independently C₁-C₆alkyl or C₃-C₁₀cycloalkyl, wherein each R⁸ is independently unsubstituted or substituted with one to three R¹¹;
each R¹¹ is independently —O—C₁-C₆alkyl; and
R¹⁴ is halo.

In some embodiments, for a compound of any one of Formulas I, I-a, IA, IB, IC, ID, IE, IF, IG, or IH:
ring A is aryl or heteroaryl;
X is —O—, —S—, —NH—, —CH═CH—, or —CH═N—;
p is 0, 1 or 2;
q is 1;
R¹ is C₁-C₆alkyl or —C(O)N(C₁-C₆alkyl)₂;
R³ is halo, —NHR⁸, —S(O)₂N(R⁷)₂, —C(O)OR⁶, —C(O)N(R⁷)₂, or heterocyclyl, wherein the heterocyclyl is independently unsubstituted or substituted with one to three R¹⁰;
each R⁴ is independently —OR⁸;
R⁶ is C₁-C₆alkyl;
each R⁷ is independently hydrogen, C₁-C₆alkyl, or C₃-C₁₀cycloalkyl, wherein each R⁷ is independently unsubstituted or substituted with one to three R¹¹;
each R⁸ is independently C₁-C₆alkyl or C₃-C₁₀cycloalkyl, wherein each R⁸ is independently unsubstituted or substituted with one to three R¹¹;
each R¹¹ is independently —O—C₁-C₆alkyl; and
R¹⁴ is halo.

In some embodiments, for a compound of any one of Formulas I, I-a, IA, IB, IC, ID, IE, IF, IG, or IH, ring A is aryl or heteroaryl. In some embodiments, ring A is aryl. In some embodiments, ring A is a monocyclic aryl. In some embodiments, ring A is a bicyclic aryl. In some embodiments, ring A is phenyl. In some embodiments, ring A is heteroaryl. In some embodiments, ring A is a monocyclic heteroaryl. In some embodiments, ring A is pyridyl. In some embodiments, ring A is a bicyclic heteroaryl. In some embodiments, ring A is a quinoline.

In some embodiments, for a compound of any one of Formulas I, I-a, IA, IB, IC, ID, IE, IF, IG, or IH, X is —CH═CH—. In some embodiments, X is —O—. In some embodiments, X is —S—. In some embodiments, X is —NH—. In some embodiments, X is —CH═N—.

In some embodiments, for a compound of any one of Formulas I, I-a, IA, IB, IC, ID, IE, IF, IG, or IH, p is 1 or 2. In some embodiments, p is 1. In some embodiments, each R⁴ is independently —OR⁸. In some embodiments, p is 1 and R⁴ is —OCH₃.

In some embodiments, for a compound of any one of Formulas I, I-a, IA, IB, IC, ID, IE, IF, IG, or IH, p is 0 or 1. In some embodiments, R⁴ is —OR⁸, —C(O)N(R⁷)₂, —NR¹²C(O)R⁸, or C₁-C₆alkyl; wherein the C₁-C₆alkyl is independently unsubstituted or substituted with one to three R¹⁰.

In some embodiments, for a compound of any one of Formulas I, I-a, IA, IB, IC, ID, IE, IF, IG, or IH, q is 1. In some embodiments, R³ is halo, —NHR⁸, —S(O)₂N(R⁷)₂, —C(O)OR⁶, —C(O)N(R⁷)₂, or heterocyclyl, wherein the heterocyclyl is independently unsubstituted or substituted with one to three R¹⁰. In some embodiments, R³ is —NHR⁸, where R⁸ is C₁-C₆alkyl or C₃-C₁₀cycloalkyl, wherein R⁸ is unsubstituted or substituted with one to three R¹¹.

In some embodiments, for a compound of any one of Formulas I, I-a, IA, IB, IC, ID, IE, IF, IG, or IH, X is —O—, —S—, or —NR⁹—. In some embodiments, X is —O—, —S—, or —NH—. In some embodiments, X is —O—. In some embodiments, X is —S—. In some embodiments, X is —NR⁹—. In some embodiments, X is —NH—.

In some embodiments, for a compound of any one of Formulas I, I-a, IA, IB, IC, ID, IE, IF, IG, or IH, X is —CR⁵═CR⁵— or —CR⁵═N—. In some embodiments, X is —CH═CH— or —CH═N—. In certain embodiments, X is —CR⁵═CR⁵—. In some embodiments, X is —CR⁵═N—.

In some embodiments, for a compound of any one of Formulas I, I-a, IA, IB, IC, ID, IE, IF, IG, or IH, R⁵ is R⁴.

In some embodiments, for a compound of any one of Formulas I, I-a, IA, IB, IC, ID, IE, IF, IG, or IH, when X is —CH═CH—, p is 1 or 2, each R⁴ is methoxy, ring A is phenyl, and q is 1, then R³ is other than adamantylamine, fluoro, or —C(O)NH-cyclopropyl. In certain embodiments, when X is —CH═CH—, p is 1 or 2, each R⁴ is methoxy, R¹ is methyl, n-butyl or —C(O)OCH₃, ring A is phenyl, and q is 1, then R³ is other than adamantylamine, fluoro, and —C(O)NH-cyclopropyl. In certain embodiments, when X is —CH═CH—, p is 1 or 2, each R⁴ is methoxy, R¹ is methyl, n-butyl or —C(O)OCH₃, R² is —CH₂Cl or C₂alkynyl, ring A is phenyl, and q is 1, then R³ is other than adamantylamine, fluoro, or —C(O)NH— cyclopropyl.

In some embodiments, for a compound of any one of Formulas I, I-a, IA, IB, IC, ID, IE, IF, IG, or IH, when X is —CR⁵═CR⁵—, p is 1 or 2, ring A is phenyl, cyclohexyl, or furyl, and q is 0 or 1, then at least one R⁴ is other than methoxy.

In some embodiments, for a compound of any one of Formulas I, I-a, IA, IB, IC, ID, IE, IF, IG, or IH, ring A is aryl or heteroaryl; q is 0, 1, or 2; and p is 1. In some embodiments, ring A is aryl or heteroaryl; q is 1; and p is 1. In some embodiments, ring A is aryl or heteroaryl; q is 1; p is 1; and X is —CH═CH—. In some embodiments, ring A is aryl or heteroaryl; q is 1; p is 1; and R³ is —NHR⁸. In some embodiments, ring A is aryl or heteroaryl; q is 1; p is 1; X is —CH═CH—; and R³ is —NHR⁸. In some embodiments, ring A is aryl or heteroaryl; q is 1; p is 1; R³ is —NHR⁸; and R¹ is C₁-C₆alkyl. In some embodiments, ring A is aryl or heteroaryl; q is 1; p is 1; X is —CH═CH—; R³ is —NHR⁸; and R¹ is C₁-C₆alkyl. In some embodiments, ring A is phenyl; q is 0, 1, or 2; and p is 1. In some embodiments, ring A is phenyl; q is 0, 1, or 2; p is 1; and X is —CH═CH—. In some embodiments, ring A is phenyl; q is 0, 1, or 2; p is 1; R³ is —NHR⁸; and R¹ is C₁-C₆alkyl. In some embodiments, ring A is phenyl; q is 0, 1, or 2; p is 1; R³ is —NHR⁸; X is —CH═CH—; and R¹ is C₁-C₆alkyl. In some embodiments, ring A is phenyl; q is 1; p is 1; R³ is —NHR⁸; X is —CH═CH—; and R¹ is C₁-C₆alkyl. In some embodiments, ring A is phenyl; q is 1; p is 1; R³ is —NHR⁸; and R¹ is C₁-C₆alkyl.

In some embodiments, the compound is a compound of Formula IJ:

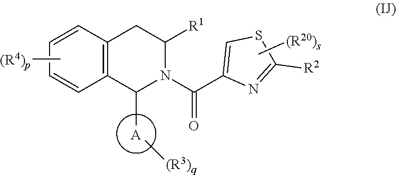

or a tautomer, stereoisomer, mixture of stereoisomers, isotopically enriched analog, or pharmaceutically acceptable salt thereof, wherein each of ring A, R¹, R², R³, R⁴, R²⁰, p, q, and s are independently as defined herein.

In some embodiments, the compound is a compound of Formula IK:

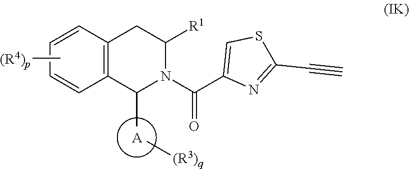

or a tautomer, stereoisomer, mixture of stereoisomers, isotopically enriched analog, or pharmaceutically acceptable salt thereof, wherein each of ring A, R¹, R³, R⁴, p, and q are independently as defined herein.

In some embodiments, the compound is a compound of Formula IL:

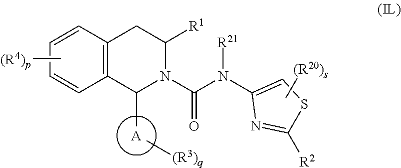

or a tautomer, stereoisomer, mixture of stereoisomers, isotopically enriched analog, or pharmaceutically acceptable salt thereof, wherein each of ring A, R¹, R², R³, R⁴, R²⁰, R²¹, p, q, and s are independently as defined herein.

In some embodiments, the compound is a compound of Formula IM:

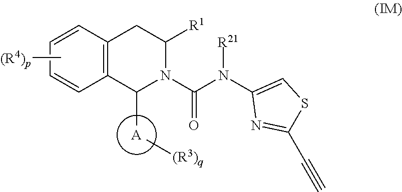

or a tautomer, stereoisomer, mixture of stereoisomers, isotopically enriched analog, or pharmaceutically acceptable salt thereof, wherein each of ring A, R¹, R³, R⁴, R²¹, p, and q are independently as defined herein.

In some embodiments, for a compound of Formula IL or IM, R²¹ is H. In some embodiments, R²¹ is C₁-C₆alkyl.

In some embodiments, for a compound of any one of Formulas IJ, IK, IL, or IM, ring A is aryl or heteroaryl. In some embodiments, ring A is aryl. In some embodiments, ring A is a monocyclic aryl. In some embodiments, ring A is a bicyclic aryl. In some embodiments, ring A is phenyl. In some embodiments, ring A is heteroaryl. In some embodiments, ring A is a monocyclic heteroaryl. In some embodiments, ring A is a bicyclic heteroaryl. In some embodiments, ring A is a quinoline.

In some embodiments, for a compound of any one of Formulas IJ, IK, IL, or IM, p is 1 or 2. In some embodiments, p is 1. In some embodiments, each R⁴ is independently —OR⁸. In some embodiments, p is 1 and R⁴ is —OCH₃.

In some embodiments, for a compound of any one of Formulas IJ, IK, IL, or IM, q is 1. In some embodiments, R³ is halo, —NHR⁸, —S(O)₂N(R⁷)₂, —C(O)OR⁶, —C(O)N(R⁷)₂, or heterocyclyl. wherein the heterocyclyl is independently unsubstituted or substituted with one to three R¹⁰. In some embodiments, R³ is —NHR⁸, where R⁸ is C₁-C₆alkyl or C₃-C₁₀cycloalkyl, wherein R⁸ is unsubstituted or substituted with one to three R¹¹.

In some embodiments, for a compound of any one of Formulas IJ, IK, IL, or IM,
ring A is aryl or heteroaryl;
p is 0, 1, or 2;
q is 1;
R¹ is C₁-C₆alkyl, —C(O)O—C₁-C₆alkyl, or —C(O)N(C₁-C₆alkyl)₂;
R³ is halo, —NHR⁸, —S(O)₂N(R⁷)₂, —C(O)OR⁶, —C(O)N(R⁷)₂, or heterocyclyl, wherein the heterocyclyl is independently unsubstituted or substituted with one to three R¹⁰;
each R⁴ is independently —OR⁸;
R⁶ is C₁-C₆alkyl;
each R⁷ is independently hydrogen, C₁-C₆alkyl, or C₃-C₁₀cycloalkyl, wherein each R⁷ is independently unsubstituted or substituted with one to three R¹¹;
each R⁸ is independently C₁-C₆alkyl or C₃-C₁₀cycloalkyl, wherein each R⁸ is independently unsubstituted or substituted with one to three R¹¹; and
each R¹¹ is independently —O—C₁-C₆alkyl.

In some embodiments, for a compound of any one of Formulas I, I-a, IA, IB, IC, ID, IE, IF, IG, IH, IJ, IK, IL, or IM, ring A, or the moiety

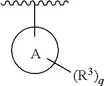

is:

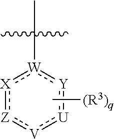

wherein 0 to 3 of U, V, W, X, Y, and Z is independently N, S, or O, and the remaining variables are CH or CR³, and each [symbol] independently represents a single or double bond, which comply with valency requirements based on U, V, W, X, Y and Z.

In some embodiments, for a compound of any one of Formulas I, I-a, IA, IB, IC, ID, IE, IF, IG, IH, IJ, IK, IL, or IM, ring A, or the moiety

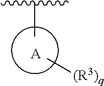

is:

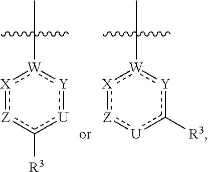

wherein 1 to 3 of U, W, X, Y, and Z is N, S, or O, and the remaining variables are CH or CR³ and [symbol] represents a single or double bond, which comply with valency requirements based on U, W, X, Y and Z.

In some embodiments, for a compound of any one of Formulas I, I-a, IA, IB, IC, ID, IE, IF, IG, IH, IJ, IK, IL, or IM, ring A is aryl or heteroaryl. In some embodiments, ring A is a monocyclic aryl or monocyclic heteroaryl. In some embodiments, ring A is heterocyclyl. In some embodiments, ring A is a 4 to 7 membered heterocyclyl. In some embodiments, ring A is aryl. In some embodiments, ring A is phenyl. In some embodiments, ring A is heteroaryl. In some embodiments, ring A is pyridyl. In some embodiments, ring A is pyrazolyl. In some embodiments, ring A is phenyl, pyridyl, piperidynyl, piperazinyl, or morpholinyl.

In some embodiments, for a compound of any one of Formulas I, I-a, IA, IB, IC, ID, IE, IF, IG, IH, IJ, IK, IL, or IM, ring A is aryl or heteroaryl, each of which is substituted by one to three R³. In some embodiments, ring A is aryl or heteroaryl, each of which is substituted by one to three R³, where at least one R³ is C₃-C₁₀cycloalkyl, heterocyclyl, aryl, or heteroaryl; wherein each C₃-C₁₀cycloalkyl, heterocyclyl, aryl, and heteroaryl of R³ is independently unsubstituted or substituted with one to three R¹⁰. In some embodiments, ring A is phenyl that is unsubstituted. In some embodiments, ring A is phenyl that is substituted by one to three R³. In some embodiments, ring A is phenyl that is substituted by one to three R³, where at least one R³ is C₃-C₁₀cycloalkyl, heterocyclyl, aryl, or heteroaryl; wherein each C₃-C₁₀cycloalkyl, heterocyclyl, aryl, and heteroaryl of R³ is independently unsubstituted or substituted with one to three R¹⁰.

In some embodiments, for a compound of any one of Formulas I, I-a, IA, IB, IC, ID, IE, IF, IG, IH, IJ, IK, IL, or IM:
ring A is aryl or heteroaryl, each of which is substituted by one to three R³, where at least one R³ is C₃-C₁₀cycloalkyl, heterocyclyl, aryl, or heteroaryl; and wherein each C₃-C₁₀cycloalkyl, heterocyclyl, aryl, and heteroaryl of R³ is independently unsubstituted or substituted with one to three R¹⁰;
each R¹⁰ is independently —OR¹², —N(R¹²)₂, —S(O)₂R¹³, —OC(O)CHR¹²N(R¹²)₂, or C₁-C₆alkyl, wherein the C₁-C₆alkyl, of R¹⁰ is independently unsubstituted or substituted with one to three R¹¹;
each R¹¹ is independently halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl, wherein the heterocyclyl is independently unsubstituted or substituted with one to three R¹⁰;
each R¹² is independently hydrogen, C₁-C₆alkyl, or C₃-C₁₀cycloalkyl; and
each R¹³ is independently C₁-C₆alkyl, or C₃-C₁₀cycloalkyl.

In some embodiments, for a compound of any one of Formulas I, I-a, IA, IB, IC, ID, IE, IF, IG, IH, IJ, IK, IL, or IM, ring A is bicyclo[1.1.1]pentan-1-yl, phenyl, piperidinyl, pyrazolyl, pyridyl, or quinolinyl, each of which is unsubstituted or substituted by one, two, or three R³. In some embodiments, ring A is bicyclo[1.1.1]pentan-1-yl, phenyl, piperidinyl, pyrazolyl, pyridyl, or quinolinyl, each of which is substituted by one, two or three R³. In some embodiments, ring A is bicyclo[1.1.1]pentan-1-yl, phenyl, piperidinyl, pyrazolyl, pyridyl, or quinolinyl, each of which is substituted by two or three R³.

In some embodiments, for a compound of any one of Formulas I, I-a, IA, IB, IC, ID, IE, IF, IG, IH, IJ, IK, IL, or IM, ring A is aryl or heteroaryl, each of which is substituted by two or three R³. In some embodiments, ring A is aryl or heteroaryl, each of which is substituted by two or three R³, wherein at least one R³ is halo.

In some embodiments, for a compound of any one of Formulas I, I-a, IA, IB, IC, ID, IE, IF, IG, IH, IJ, IK, IL, or IM, ring A is cyclohexyl. In some embodiments, ring A is C₄-C₁₀cycloalkyl. In some embodiments, ring A is a C₄-C₇cycloalkyl. In some embodiments, ring A is bicyclo[1.1.1]pentanyl. In some embodiments, ring A is selected from cyclobutyl, cyclopentyl, cyclohexyl, and cycloheptyl.

In some embodiments, for a compound of any one of Formulas I, I-a, IA, IB, IC, ID, IE, IF, IG, IH, IJ, IK, IL, or IM, ring A, or the moiety

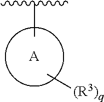

is:

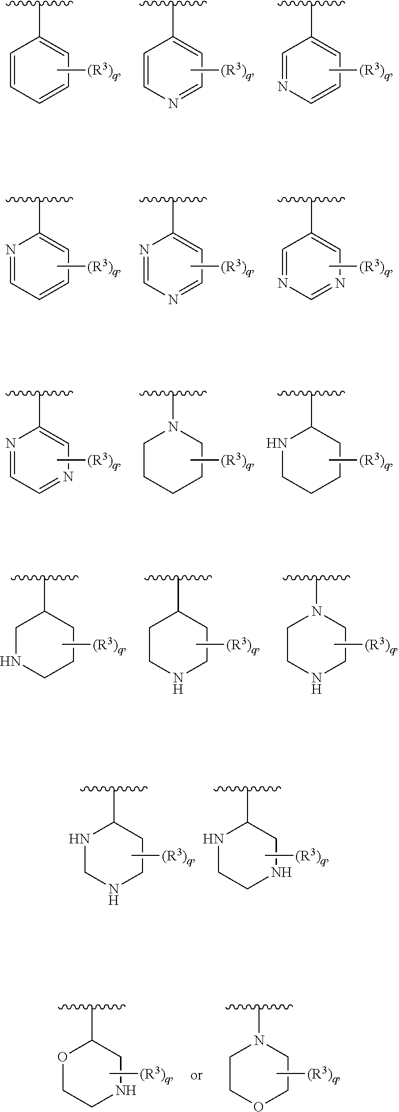

where q and each R³ is independently as defined herein.

In some embodiments, for a compound of any one of Formulas I, I-a, IA, IB, IC, ID, IE, IF, IG, IH, IJ, IK, IL, or IM, ring A, or the moiety

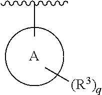

is:

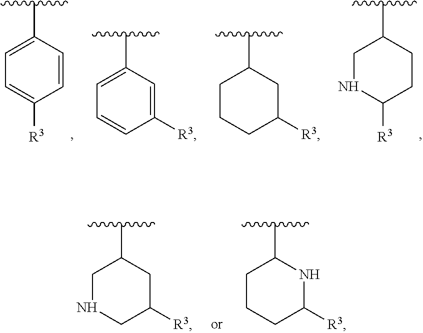

where R³ is independently as defined herein.

In some embodiments, for a compound of any one of Formulas I, I-a, IA, IB, IC, ID, IE, IF, IG, IH, IJ, IK, IL, or IM, ring A is a bridged bicyclic ring selected from:

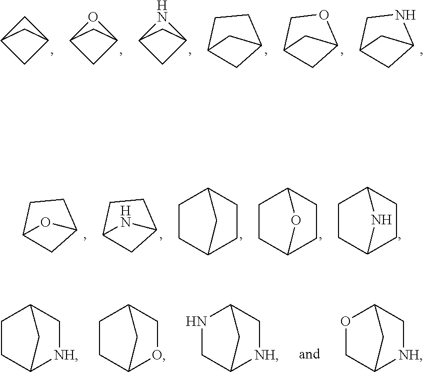

wherein each is substituted with one to three R³. In certain embodiments, ring A is a bridged bicyclic ring selected from:

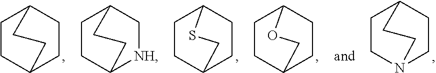

wherein each R³ is attached to a carbon atom on the bridged bicyclic ring.

In some embodiments, for a compound of any one of Formulas I, I-a, IA, IB, IC, ID, IE, IF, IG, IH, IJ, IK, IL, or IM, ring A, or the moiety

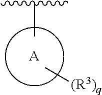

is:

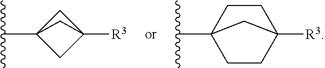

In some embodiments, the compound is a compound of Formula IN:

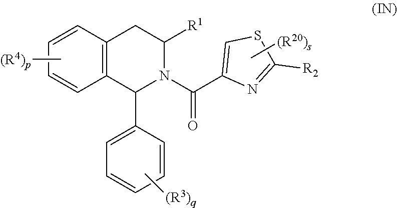

or a tautomer, stereoisomer, mixture of stereoisomers, isotopically enriched analog, or pharmaceutically acceptable salt thereof, wherein each of R¹, R², R³, R⁴, R²⁰, p, q, and s are independently as defined herein.

In some embodiments, the compound is a compound of Formula IP:

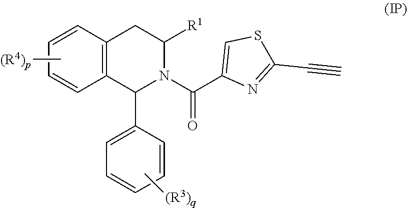

or a tautomer, stereoisomer, mixture of stereoisomers, isotopically enriched analog, or pharmaceutically acceptable salt thereof, wherein each of R¹, R³, R⁴, p, and q are independently as defined herein.

In some embodiments, the compound is a compound of Formula IQ:

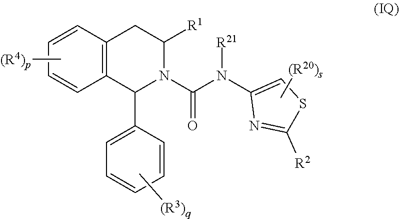

or a tautomer, stereoisomer, mixture of stereoisomers, isotopically enriched analog, or pharmaceutically acceptable salt thereof, wherein each of R¹, R², R³, R⁴, R²⁰, R²¹, p, q, and s are independently as defined herein.

In some embodiments, the compound is a compound of Formula IR:

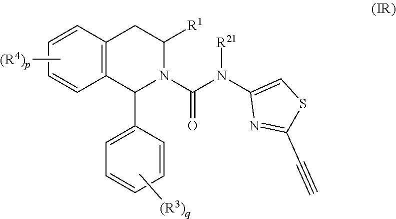

or a tautomer, stereoisomer, mixture of stereoisomers, isotopically enriched analog, or pharmaceutically acceptable salt thereof, wherein each of R¹, R³, R⁴, R²¹, p, and q are independently as defined herein.

In some embodiments, for a compound of Formula IQ or IR, R²¹ is H. In some embodiments, R²¹ is C₁-C₆alkyl.

In some embodiments, for a compound of any one of Formulas I, I-a, IA, IB, IC, ID, IE, IF, IG, IH, IJ, IK, IL, IM, IN, IP, IQ, or IR, R¹ is C₁-C₆alkyl, C₂-C₆alkenyl, C₂-C₆alkynyl, C₁-C₆haloalkyl, C₃-C₁₀cycloalkyl, heteroaryl, —CN, —C(O)OR⁶, —C(O)N(R⁷)₂, —N(R⁷)₂, —OR⁷, or —C₁-C₆alkyl-OR⁷; wherein the heteroaryl is unsubstituted or substituted with C₁-C₆alkyl.

In some embodiments, for a compound of any one of Formulas I, I-a, IA, IB, IC, ID, IE, IF, IG, IH, IJ, IK, IL, IM, IN, IP, IQ, or IR, R¹ is C₂-C₆alkyl, C₂-C₆alkenyl, C₂-C₆alkynyl, C₁-C₆haloalkyl, C₃-C₁₀cycloalkyl, heteroaryl, —CN, —C(O)OR⁶, —C(O)N(R⁷)₂, —N(R⁷)₂, —OR⁷, or —C₁-C₆alkyl-OR⁷; wherein the heteroaryl is unsubstituted or substituted with C₁-C₆alkyl.

In some embodiments, for a compound of any one of Formulas I, I-a, IA, IB, IC, ID, IE, IF, IG, IH, IJ, IK, IL, IM, IN, IP, IQ, or IR, R¹ is C₂-C₆alkyl, C₂-C₆alkenyl, C₂-C₆alkynyl, C₁-C₆haloalkyl, C₃-C₁₀cycloalkyl, heteroaryl, —CN, —C(O)N(R⁷)₂, —N(R⁷)₂, —OR⁷, or —C₁-C₆alkyl-OR⁷; wherein the heteroaryl is unsubstituted or substituted with C₁-C₆alkyl. In some embodiments, R¹ is C₂-C₆alkyl, C₂-C₆alkenyl, C₂-C₆alkynyl, C₁-C₆haloalkyl, C₃-C₁₀cycloalkyl, 5-membered heteroaryl, —CN, —C(O)N(R⁷)₂, —N(R⁷)₂, —OR⁷, or —C₁-C₆alkyl-OR⁷; wherein the 5-membered heteroaryl is unsubstituted or substituted with C₁-C₆alkyl.

In some embodiments, for a compound of any one of Formulas I, I-a, IA, IB, IC, ID, IE, IF, IG, IH, IJ, IK, IL, IM, IN, IP, IQ, or IR, R¹ is C₁-C₆alkyl, C₂-C₆alkenyl, C₂-C₆alkynyl, C₁-C₆haloalkyl, C₃-C₁₀cycloalkyl, —CN, —C(O)OR⁶, —C(O)N(R⁷)₂, —N(R⁷)₂, —OR⁷, or —C₁-C₆alkyl-OR⁷. In some embodiments, R¹ is —C(O)OR⁶ or —C(O)N(R⁷)₂. In some embodiments, R is C₁-C₆alkyl. In some embodiments, R¹ is C₂-C₆alkyl. In some embodiments, R¹ is C₃-C₆alkyl. In some embodiments, R¹ is C₅-C₆alkyl. In some embodiments, R¹ is C₂-C₃alkyl. In some embodiments, R¹ is C₄-C₆alkyl. In some embodiments, R¹ is methyl. In some embodiments, R¹ is n-butyl. In some embodiments, R¹ is —CH₂—R¹⁶, wherein R¹⁶ is C₁-C₅alkyl, C₂-C₅alkenyl, C₂-C₅alkynyl, C₁-C₅haloalkyl, or —C₁-C₅alkyl-OR⁷. In some embodiments, R¹ is C₂-C₆alkenyl, C₂-C₆alkynyl, C₁-C₆haloalkyl, C₃-C₁₀cycloalkyl, —CN, —OR⁷, —C(O)N(R⁷)₂, —OC(O)R⁶, —S(O)₂R⁸, —S(O)₂N(R⁷)₂, —S(O)N(R⁷)₂, —S(O)R⁸, —N(R⁷)₂, —NO₂, —C₁-C₆alkyl-OR⁷, or —Si(R¹⁵)₃.

In some embodiments, for a compound of any one of Formulas I, I-a, IA, IB, IC, ID, IE, IF, IG, IH, IJ, IK, IL, IM, IN, IP, IQ, or IR, R¹ is other than methyl. In certain embodiments, R¹ is other than n-butyl. In certain embodiments, R¹ is other than —C(O)OR⁶. In certain embodiments, R¹ is other than —C(O)OCH₃.

In some embodiments, for a compound of any one of Formulas I, I-a, IA, IB, IC, ID, IE, IF, IG, IH, IJ, IK, IL, IM, IN, IP, IQ, or IR, q is 1, 2, or 3 and at least one R³ is halo, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)R⁸, —C(O)R⁶, or —OC(O)CHR⁸N(R¹²)₂. In certain embodiments, q is 1, 2, or 3 and at least one R³ is halo.

In some embodiments, for a compound of any one of Formulas I, I-a, IA, IB, IC, ID, IE, IF, IG, IH, IJ, IK, IL, IM, IN, IP, IQ, or IR, q is 1, 2, or 3 and at least one R³ is —NHR⁸. In certain embodiments, q is 1, 2, or 3 and at least one R³ is —N(R⁸)₂. In certain embodiments, q is 2, and one R³ is halo and the other R³ is —N(R⁸)₂. In certain embodiments, q is 3, and two R³ are independently halo and one R³ is —N(R⁸)₂. In some embodiments, q is 1, 2, or 3 and at least one R³ is —NHR⁸, wherein R⁸ is adamantyl or adamantyl-(C₁-C₆aliphatic).

In some embodiments, for a compound of any one of Formulas I, I-a, IA, IB, IC, ID, IE, IF, IG, IH, IJ, IK, IL, IM, IN, IP, IQ, or IR, q is 1, 2, or 3 and at least one R³ is —C(O)OR⁶ or —C(O)R⁶. In certain embodiments, q is 1, 2, or 3 and at least one R³ is —S(O)₂N(R⁷)₂, —S(O)N(R⁷)₂, or —C(O)N(R⁷)₂. In certain embodiments, q is 1, 2, or 3 and at least one R³ is —S(O)₂R⁸, —S(O)R⁸, —NR¹²C(O)R⁸, —NR¹²C(O)OR⁸, —OC(O)R⁸, or —OC(O)CHR⁸N(R¹²)₂.

In some embodiments, for a compound of any one of Formulas I, I-a, IA, IB, IC, ID, IE, IF, IG, IH, IJ, IK, IL, IM, IN, IP, IQ, or IR, q is 1, 2, or 3 and each R³ is independently halo, —CN, —OR⁸, —NHR⁸, —S(O)₂R⁸, —S(O)₂N(R⁷)₂, —NO₂, —Si(R²)₃, —SF₅, —C(O)OR⁶, —C(O)N(R⁷)₂, —NR²C(O)R⁸, —NR²C(O)OR⁸, —OC(O)R⁸, —OC(O)CHR⁸N(R¹²)₂, C₁-C₆alkyl, C₃-C₁₀cycloalkyl, heterocyclyl, heteroaryl, or —C₁-C₆alkylheterocyclyl; wherein each C₁-C₆alkyl, C₃-C₁₀cycloalkyl, heterocyclyl, heteroaryl, or —C₁-C₆alkylheterocyclyl of R³ is independently unsubstituted or substituted with one to three R¹¹.

In some embodiments, for a compound of any one of Formulas I, I-a, IA, IB, IC, ID, IE, IF, IG, IH, IJ, IK, IL, IM, IN, IP, IQ, or IR, q is 1, 2, or 3 and each R³ is independently halo, —CN, —OR⁸, —NHR⁸, —S(O)₂R⁸, —S(O)₂N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)R⁸, —OC(O)CHR⁸N(R¹²)₂, C₁-C₆alkyl, C₃-C₁₀cycloalkyl, heterocyclyl, heteroaryl, or —C₁-C₆alkylheterocyclyl; wherein each C₁-C₆alkyl, C₃-C₁₀cycloalkyl, heterocyclyl, heteroaryl, or —C₁-C₆alkylheterocyclyl is independently unsubstituted or substituted with one to three substituents independently selected from —OR¹², —N(R¹²)₂, —S(O)₂R¹³, —OC(O)CHR¹²N(R¹²)₂, and C₁-C₆alkyl optionally substituted with one to three halo, —OR¹², —N(R¹²)₂, —Si(R¹²)₃, —C(O)OR¹², —NR²C(O)OR¹², —OC(O)CHR¹²N(R¹²)₂, C₁-C₆alkyl, or heterocyclyl; wherein each R¹² is independently hydrogen, C₁-C₆alkyl or C₃-C₁₀cycloalkyl; and each R¹³ is independently C₁-C₆alkyl or C₃-C₁₀cycloalkyl.

In some embodiments, for a compound of any one of Formulas I, I-a, IA, IB, IC, ID, IE, IF, IG, IH, IJ, IK, IL, IM, IN, IP, IQ, or IR, q is 1, 2, or 3 and each R³ is independently —NH₂, fluoro, methyl, pyridine-4-carboxamido, pyridin-3-amino, pentyloxycarbonylamino, N-(3-aminobicyclo[1.1.1]pentan-1-yl)amino, morpholin-4-yl, methoxycarbonyl, dimethylcarbamoyl, cyclopropylcarbamoyl, cyclohexyl, cyclobutylcarbamoyl, cyclobutylaminosulfonyl, adamantylanmino, (adamantan-1-ylanino)methyl, 3-methyl-1,2,4-oxadiazol-5-yl, 2-methylpyridine-4-carboxamido, (bicyclo[1.1.1]pentan-1-ylamino)methyl, (adamantan-1-yl)carbamoyl, or (2-methoxyethyl)carbamoyl.

In some embodiments, for a compound of any one of Formulas I, I-a, IA, IB, IC, ID, IE, IF, IG, IH, IJ, IK, IL, IM, IN, IP, IQ, or IR, q is 1, and R³ is —NH₂, fluoro, methyl, pyridine-4-carboxamido, pyridin-3-amino, pentyloxycarbonylamino, N-(3-aminobicyclo[1.1.1]pentan-1-yl)amino, morpholin-4-yl, methoxycarbonyl, dimethylcarbamoyl, cyclopropylcarbamoyl, cyclohexyl, cyclobutylcarbamoyl, cyclobutylaminosulfonyl, adamantylamino, (adamantan-1-ylamino)methyl, 3-methyl-1,2,4-oxadiazol-5-yl, 2-methylpyridine-4-carboxamido, (bicyclo[1.1.1]pentan-1-ylamino)methyl, (adamantan-1-yl)carbamoyl, or (2-methoxyethyl)carbamoyl.

In some embodiments, for a compound of any one of Formulas I, I-a, IA, IB, IC, ID, IE, IF, IG, IH, IJ, IK, IL, IM, IN, IP, IQ, or IR, p is 1, 2, or 3 and each R⁴ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, C₁-C₆alkyl, C₂-C₆alkenyl, C₂-C₆alkynyl, or C₃-C₁₀cycloalkyl; wherein each C₁-C₆alkyl, C₂-C₆alkenyl, C₂-C₆alkynyl, or C₃-C₁₀cycloalkyl of R⁴ is independently unsubstituted or substituted with one to three R¹⁰.

In some embodiments, for a compound of any one of Formulas I, I-a, IA, IB, IC, ID, IE, IF, IG, IH, IJ, IK, IL, IM, IN, IP, IQ, or IR, p is 1, 2, or 3 and each R⁴ is independently halo, —CN, —OR⁷, C₁-C₆alkyl, C₂-C₆alkynyl, or C₃-C₁₀cycloalkyl; wherein each C₁-C₆alkyl, C₂-C₆alkynyl, or C₃-C₁₀cycloalkyl of R⁴ is independently unsubstituted or substituted with one to three R¹⁰. In certain embodiments, p is 1, 2, or 3 and each R⁴ is independently halo, —CN, —OH, C₁-C₆alkyl, C₂-C₆alkynyl, or C₃-C₁₀cycloalkyl. In certain embodiments, p is 1, 2, or 3 and each R⁴ is independently halo, —CN, —OH, —OR⁸, C₁-C₆alkyl, or C₂-C₆alkynyl; wherein the C₁-C₆alkyl of R⁴ is unsubstituted or substituted with one to three R¹⁰.

In some embodiments, for a compound of any one of Formulas I, I-a, IA, IB, IC, ID, IE, IF, IG, IH, IJ, IK, IL, IM, IN, IP, IQ, or IR, p is 1, 2, or 3 and each R⁴ is independently halo, —CN, —OH, —OR⁸, C₁-C₆alkyl, C₂-C₆alkynyl; wherein the C₁-C₆alkyl of R⁴ is unsubstituted or substituted with one to three substituents independently selected from —OR¹², —N(R¹²)₂, —S(O)₂R¹³, —OC(O)CHR¹²N(R¹²)₂, and C₁-C₆alkyl optionally substituted with one to three halo, —OR¹², —N(R¹²)₂, —Si(R¹²)₃, —C(O)OR¹², —NR¹²C(O)OR², —OC(O)CHR¹²N(R¹²)₂, C₁-C₆alkyl, or heterocyclyl; wherein each R¹² is independently hydrogen, C₁-C₆alkyl or C₃-C₁₀cycloalkyl; and each R¹³ is independently C₁-C₆alkyl or C₃-C₁₀cycloalkyl.

In some embodiments, for a compound of any one of Formulas I, I-a, IA, IB, IC, ID, IE, IF, IG, IH, IJ, IK, IL, IM, IN, IP, IQ, or IR, each R⁵ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, C₁-C₆alkyl, C₂-C₆alkenyl, C₂-C₆alkynyl, or C₃-C₁₀cycloalkyl; wherein each C₁-C₆alkyl, C₂-C₆alkenyl, C₂-C₆alkynyl, or C₃-C₁₀cycloalkyl of R⁵ is independently unsubstituted or substituted with one to three R¹⁰. In certain embodiments, each R⁵ is independently halo, —CN, —OR⁷, C₁-C₆alkyl, C₂-C₆alkynyl, or C₃-C₁₀cycloalkyl; wherein each C₁-C₆alkyl, C₂-C₆alkynyl, or C₃-C₁₀cycloalkyl of R⁵ is independently unsubstituted or substituted with one to three R¹⁰. In certain embodiments, each R⁵ is independently halo, —CN, —OH, C₁-C₆alkyl, C₂-C₆alkynyl, or C₃-C₁₀cycloalkyl. In certain embodiments, each R⁵ is independently halo, —CN, —OH, —OR⁸, C₁-C₆alkyl, or C₂-C₆alkynyl; wherein the C₁-C₆alkyl of R⁵ is unsubstituted or substituted with one to three R¹⁰. In certain embodiments, each R⁵ is independently halo, —CN, —OH, —OR⁸, C₁-C₆alkyl, C₂-C₆alkynyl; wherein the C₁-C₆alkyl of R⁵ is unsubstituted or substituted with one to three substituents independently selected from —OR¹², —N(R¹²)₂, —S(O)₂R¹³, —OC(O)CHR¹²N(R¹²)₂, and C₁-C₆alkyl that is unsubstituted or substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl; wherein each R¹² is independently hydrogen, C₁-C₆alkyl or C₃-C₁₀cycloalkyl; and each R¹³ is independently C₁-C₆alkyl or C₃-C₁₀cycloalkyl.

In some embodiments, for a compound of any one of Formulas I, I-a, IA, IB, IC, ID, IE, IF, IG, IH, IJ, IK, IL, IM, IN, IP, IQ, or IR, each R⁶ is independently hydrogen, C₁-C₆alkyl, C₂-C₆alkenyl, or —C₁-C₆alkylC₃-C₁₀cycloalkyl; wherein each R⁶ is independently unsubstituted or substituted with one to three R¹¹.

In some embodiments, for a compound of any one of Formulas I, I-a, IA, IB, IC, ID, IE, IF, IG, IH, IJ, IK, IL, IM, IN, IP, IQ, or IR, each R⁶ is independently hydrogen, C₁-C₆alkyl, C₂-C₆alkenyl, or —C₁-C₆alkylC₃-C₁₀cycloalkyl; wherein each R⁶ is independently unsubstituted or substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl; wherein each R¹² is independently hydrogen, C₁-C₆alkyl or C₃-C₁₀cycloalkyl. In certain embodiments, each R⁷ is independently hydrogen, C₁-C₆alkyl, C₃-C₁₀cycloalkyl, heterocyclyl, heteroaryl, —C₁-C₆alkylC₃-C₆cycloalkyl, —C₁-C₆alkylheterocyclyl, or two R⁷ together with the nitrogen atom to which they are attached, form a 4 to 7 membered heterocyclyl; wherein each R⁷ or ring formed thereby is independently unsubstituted or substituted with one to three R¹¹. In certain embodiments, each R⁷ is independently hydrogen, C₁-C₆alkyl, C₃-C₁₀cycloalkyl, heterocyclyl, heteroaryl, —C₁-C₆alkylC₃-C₆cycloalkyl, —C₁-C₆alkylheterocyclyl, or two R⁷ together with the nitrogen atom to which they are attached, form a 4 to 7 membered heterocyclyl; wherein each R⁷ or ring formed thereby is independently unsubstituted or substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl; wherein each R¹² is independently hydrogen, C₁-C₆alkyl, or C₃-C₁₀cycloalkyl.

In some embodiments, for a compound of any one of Formulas I, I-a, IA, IB, IC, ID, IE, IF, IG, IH, IJ, IK, IL, IM, IN, IP, IQ, or IR, each R¹ is independently C₁-C₆alkyl, C₂-C₆alkynyl, C₃-C₁₀cycloalkyl, —C₁-C₆alkylC₃-C₁₀cycloalkyl, or —C₁-C₆alkylaryl: wherein each R¹ is independently unsubstituted or substituted with one to three R¹¹. In certain embodiments, each R⁸ is independently C₁-C₆alkyl, C₂-C₆alkynyl, C₃-C₁₀cycloalkyl, —C₁-C₆alkylC₃-C₁₀cycloalkyl, or —C₁-C₆alkylaryl; wherein each R⁸ is independently unsubstituted or substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl; wherein each R¹² is independently hydrogen, C₁-C₆alkyl, or C₃-C₁₀cycloalkyl.

In some embodiments, for a compound of any one of Formulas I, I-a, IA, IB, IC, ID, IE, IF, IG, IH, IJ, IK, IL, IM, IN, IP, IQ, or IR, each R¹⁰ is independently —OR¹², —N(R¹²)₂, —S(O)₂R¹³, —OC(O)CHR¹²N(R¹)₂, or C₁-C₆alkyl, wherein the C₁-C₆alkyl, of R¹⁰ is independently unsubstituted or substituted with one to three R¹¹; each R¹¹ is independently halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl; each R¹² is independently hydrogen, C₁-C₆alkyl, or C₃-C₁₀cycloalkyl; and each R¹³ is independently C₁-C₆alkyl or C₃-C₁₀cycloalkyl.

In some embodiments, for a compound of any one of Formulas I, I-a, IA, IB, IC, ID, IE, IF, IG, IH, IJ, IK, IL, IM, IN, IP, IQ, or IR, each R¹⁵ is independently C₁-C₆alkyl.

In some embodiments, for a compound of any one of Formulas I, I-a, IA, IB, IC, ID, IE, IF, IG, IH, IJ, IK, IL, IM, IN, IP, IQ, or IR, p is 0. In certain embodiments, p is 0 or 1. In certain embodiments, p is 1 or 2. In certain embodiments, p is 1. In certain embodiments, p is 2.

In some embodiments, for a compound of any one of Formulas I, I-a, IA, IB, IC, ID, IE, IF, IG, IH, IJ, IK, IL, IM, IN, IP, IQ, or IR, q is 0. In certain embodiments, q is 0 or 1. In certain embodiments, q is 1 or 2. In certain embodiments, q is 1. In certain embodiments, q is 2. In certain embodiments, q is 3.

In another aspect, the present disclosure provides a compound of Formula II:

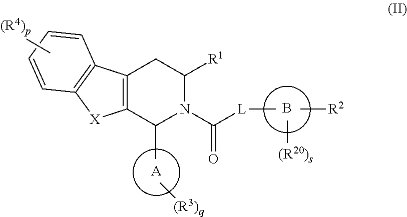

or a tautomer, stereoisomer, mixture of stereoisomers, isotopically enriched analog, or pharmaceutically acceptable salt thereof, wherein:
ring A is C₄-C₁₀cycloalkyl, heterocyclyl, aryl, or heteroaryl;
X is —NR⁵—, —O—, or —S—;
L is absent or —NR⁹—;
p is 0, 1, 2, or 3;
q is 0, 1, 2, or 3;
s is 0, 1, 2, or 3;
R¹ is C₁-C₆alkyl, C₂-C₆alkenyl, C₂-C₆alkynyl, C₁-C₆haloalkyl, C₃-C₁₀cycloalkyl, —CN, —OH, —C(O)OR⁶, —C(O)N(R⁷)₂, —OC(O)R⁶, —S(O)₂R⁸, —S(O)₂N(R⁷)₂, —S(O)N(R⁷)₂, —S(O)R⁸, —NH₂, —NHR⁸, —N(R⁸)₂, —NO₂, —OR⁸, —C₁-C₆alkyl-OH, —C₁-C₆alkyl-OR⁸, or —Si(R¹⁵)₃; R² is C₂alkynyl that is unsubstituted or substituted with C₁-C₆alkyl, C₂-C₆alkenyl, C₂-C₆alkynyl, C₃-C₁₀cycloalkyl, C₆-C₁₄aryl, C₇-C₁₅aralkyl, heteroaryl, or heterocyclyl;
each R³ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)N(R⁷)₂, —OC(O)R⁸, —C(O)R⁶, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R³ is independently unsubstituted or substituted with one to three R¹⁰; each R⁴ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R⁴ is independently unsubstituted or substituted with one to three R¹⁰;
R⁵ is hydrogen or C₁-C₆alkyl;
each R⁶ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each R⁶ is independently unsubstituted or substituted with one to three R¹¹;
each R⁷ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₆cycloalkyl, —C₂-C₆alkenylC₃-C₆cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, —C₂-C₆alkenylheteroaryl, or two R⁷ together with the nitrogen atom to which they are attached, form a 4 to 7 membered heterocyclyl; wherein each R⁷ or ring formed thereby is independently unsubstituted or substituted with one to three R¹¹;
each R⁸ is independently C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, or —C₂-C₆alkenylheteroaryl; wherein each R⁸ is independently unsubstituted or substituted with one to three R¹¹;
R⁹ is hydrogen or C₁-C₆alkyl;
each R¹⁰ is independently halo, —CN, —OR¹², —NO₂, —N(R¹²)₂, —S(O)R¹³, —S(O)₂R¹³, —S(O)N(R¹²)₂, —S(O)₂N(R²)₂, —Si(R²)₃, —C(O)R², —C(O)OR², —C(O)N(R²)₂, —NR²C(O)R², —OC(O)R², —OC(O)OR², —OC(O)N(R¹²)₂, —NR¹²C(O)OR¹², —OC(O)CHR¹²N(R¹²)₂, —P(O)(OR¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl, wherein each C₁-C₆alkyl, C₁-C₆haloalkyl, C₂-C₆alkenyl, C₂-C₆alkynyl, C₃-C₁₀cycloalkyl, heterocyclyl, aryl, or heteroaryl of R^{1′} is independently unsubstituted or substituted with one to three R¹¹;
each R¹¹ is independently halo, —CN, —OR¹², —NO₂, —N(R¹²)₂, —S(O)R¹³, —S(O)₂R¹³, —S(O)N(R¹²)₂, —S(O)₂N(R²)₂, —Si(R²)₃, —C(O)R², —C(O)OR², —C(O)N(R²)₂, —NR²C(O)R², —OC(O)R², —OC(O)OR², —OC(O)N(R¹²)₂, —NR¹²C(O)OR¹², —OC(O)CHR¹²N(R¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl;
each R¹² is independently hydrogen, C₁-C₆alkyl, or C₃-C₁₀cycloalkyl;
each R¹³ is independently C₁-C₆alkyl, or C₃-C₁₀cycloalkyl;
each R¹⁵ is independently C₁-C₆alkyl, C₂-C₆alkenyl, aryl, heteroaryl, —C₁-C₆alkylaryl, —C₂-C₆alkenylaryl, —C₁-C₆alkylheteroaryl, and —C₂-C₆alkenylheteroaryl; and
each R²⁰ is independently halogen, C₁-C₆alkyl, or C₂-C₆alkenyl.

In some embodiments, for a compound of Formula II, ring B includes a single heteroatom. In some embodiments, ring B includes at least one heteroatom, such as 1, 2, or 3 heteroatoms. In some embodiments, ring B includes 2 heteroatoms. In some embodiments, ring B includes one or more heteroatoms selected from S, O, and N. In some embodiments, ring B includes a heteroatom that is N. In some embodiments, ring B includes a heteroatom that is S. In some embodiments, ring B includes heteroatoms that are N and S. In some embodiments, ring B is a bicyclic heteroaryl. In some embodiments, ring B is a monocyclic heteroaryl. In some embodiments, ring B is a 5-10 membered heteroaryl. In some embodiments, ring B is a 5-6 membered heteroaryl. In some embodiments, ring B is a 5-6 membered heteroaryl including at least one heteroatom selected from S, O, and N. In some embodiments, ring B has the structure:

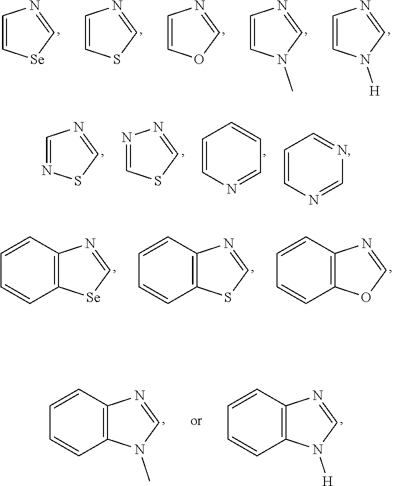

which structure is substituted with R² and s R²⁰s. In some embodiments, ring B has a structure shown above and is substituted with R² and 0 R²⁰s (e.g., s is 0). In some embodiments, ring B has the structure

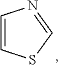

which structure is substituted with R² and s R²⁰s.

In some embodiments, the compound is a compound of Formula IIA:

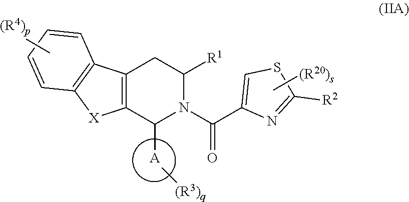

or a tautomer, stereoisomer, mixture of stereoisomers, isotopically enriched analog, or pharmaceutically acceptable salt thereof, where each of ring A, X, R¹, R², R³, R⁴, R²⁰, p, q, and s are as defined herein.

In some embodiments, the compound is a compound of Formula IIB:

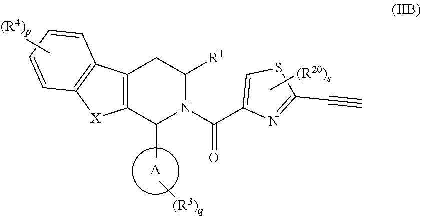

or a tautomer, stereoisomer, mixture of stereoisomers, isotopically enriched analog, or pharmaceutically acceptable salt thereof, where each of ring A, X, R¹, R³, R⁴, R²⁰, p, q, and s are as defined herein.

In some embodiments, the compound is a compound of Formula IIC:

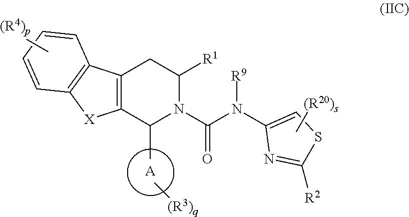

or a tautomer, stereoisomer, mixture of stereoisomers, isotopically enriched analog, or pharmaceutically acceptable salt thereof, wherein each of ring A, X, R¹, R², R³, R⁴, R⁹, R²⁰, p, q, and s are independently as defined herein.

In some embodiments, the compound is a compound of Formula IID:

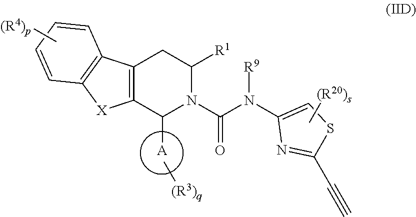

or a tautomer, stereoisomer, mixture of stereoisomers, isotopically enriched analog, or pharmaceutically acceptable salt thereof, wherein each of ring A, X, R¹, R³, R⁴, R⁹, R²⁰, p, q, and s are independently as defined herein.

In some embodiments, for a compound of any one of Formulas II, IIA, IIB, IIC, or IID, X is NR⁵. In some embodiments, X is NH. In some embodiments, X is O. In some embodiments, X is S.

In some embodiments, the compound is a compound of Formula IIE:

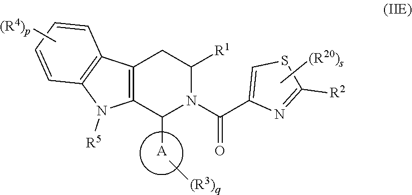

or a tautomer, stereoisomer, mixture of stereoisomers, isotopically enriched analog, or pharmaceutically acceptable salt thereof, where each of ring A, R¹, R², R³, R⁴, R⁵, R²⁰, p, q, and s are as defined herein.

In some embodiments, the compound is a compound of Formula IIF:

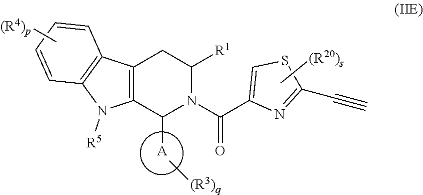

or a tautomer, stereoisomer, mixture of stereoisomers, isotopically enriched analog, or pharmaceutically acceptable salt thereof, where each of ring A, R¹, R³, R⁴, R⁵, R²⁰, p, q, and s are as defined herein.

In some embodiments, the compound is a compound of Formula IIG:

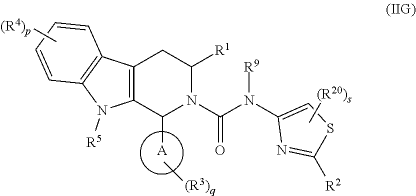

or a tautomer, stereoisomer, mixture of stereoisomers, isotopically enriched analog, or pharmaceutically acceptable salt thereof, wherein each of ring A, R¹, R², R³, R⁴, R⁵, R⁹, R²⁰, p, q, and s are independently as defined herein.

In some embodiments, the compound is a compound of Formula IIH:

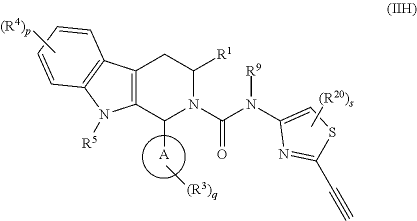

or a tautomer, stereoisomer, mixture of stereoisomers, isotopically enriched analog, or pharmaceutically acceptable salt thereof, wherein each of ring A, X, R¹, R³, R⁴, R⁹, R²⁰, p, q, and s are independently as defined herein.

In some embodiments, for a compound of any one of Formulas IIC, IID, IIG, or IIH, R⁹ is H. In some embodiments, R⁹ is C₁-C₆alkyl.

In some embodiments, for a compound of any one of Formulas IIE, IIF, IG, or IIH, R⁵ is H. In some embodiments, R⁵ is C₁-C₆alkyl.

In some embodiments, for a compound of any one of Formulas II, IIA, IIB, IIC, IID, IIE, IIF, IG, or IIH, s is 0. In some embodiments, s is 1. In some embodiments, s is 2. In some embodiments, s is 3.

In some embodiments, for a compound of any one of Formulas II, IIA, IIB, IIC, IID, IIE, IIF, IG, or IIH, ring A is aryl or heteroaryl. In some embodiments, ring A is aryl. In some embodiments, ring A is a monocyclic aryl. In some embodiments, ring A is a bicyclic aryl. In some embodiments, ring A is phenyl. In some embodiments, ring A is heteroaryl. In some embodiments, ring A is a monocyclic heteroaryl. In some embodiments, ring A is a bicyclic heteroaryl. In some embodiments, ring A is a quinoline.

In some embodiments, for a compound of any one of Formulas II, IIA, IIB, IIC, IID, IIE, IIF, IG, or IIH, ring A, or the moiety

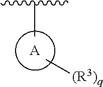

is:

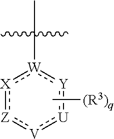

wherein 0 to 3 of U, V, W, X, Y, and Z is independently N, S, or O, and the remaining variables are CH or CR³, and each [symbol] independently represents a single or double bond, which comply with valency requirements based on U, V, W, X, Y and Z.

In some embodiments, for a compound of any one of Formulas II, IIA, IIB, IIC, IID, IIE, IIF, IIG, or IIH, ring A, or the moiety

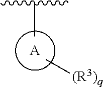

is:

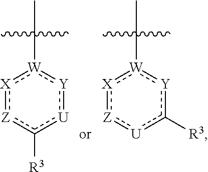

wherein 1 to 3 of U, W, X, Y, and Z is N, S, or O, and the remaining variables are CH or CR³ and [symbol] represents a single or double bond, which comply with valency requirements based on U, W, X, Y and Z.

In some embodiments, for a compound of any one of Formulas II, IIA, IIB, IIC, IID, IIE, IIF, IIG, or IIH, ring A is aryl or heteroaryl. In some embodiments, ring A is a monocyclic aryl or monocyclic heteroaryl. In some embodiments, ring A is heterocyclyl. In some embodiments, ring A is a 4 to 7 membered heterocyclyl. In some embodiments, ring A is aryl. In some embodiments, ring A is phenyl. In some embodiments, ring A is heteroaryl. In some embodiments, ring A is pyridyl. In some embodiments, ring A is pyrazolyl. In some embodiments, ring A is phenyl, pyridyl, piperidynyl, piperazinyl, or morpholinyl.

In some embodiments, for a compound of any one of Formulas II, IIA, IIB, IIC, IID, IIE, IIF, IIG, or IIH, ring A is aryl or heteroaryl, each of which is substituted by one to three R³. In some embodiments, ring A is aryl or heteroaryl, each of which is substituted by one to three R³, where at least one R³ is C₃-C₁₀cycloalkyl, heterocyclyl, aryl, or heteroaryl; wherein each

C₃-C₁₀cycloalkyl, heterocyclyl, aryl, and heteroaryl of R³ is independently unsubstituted or substituted with one to three R¹⁰. In some embodiments, ring A is phenyl that is unsubstituted. In some embodiments, ring A is phenyl that is substituted with one to three R¹⁰. In some embodiments, ring A is phenyl that is unsubstituted. In some embodiments, ring A is phenyl that is substituted with one to three R¹⁰, where at least one R³ is C₃-C₁₀cycloalkyl, heterocyclyl, aryl, or heteroaryl; wherein each C₃-C₁₀cycloalkyl, heterocyclyl, aryl, and heteroaryl of R³ is independently unsubstituted or substituted with one to three R¹⁰.

In some embodiments, for a compound of any one of Formulas II, IIA, IIB, IIC, IID, IIE, IIF, IIG, or IIH:
ring A is aryl or heteroaryl, each of which is substituted by one to three R³, where at least one R³ is C₃-C₁₀cycloalkyl, heterocyclyl, aryl, or heteroaryl; and wherein each C₃-C₁₀cycloalkyl, heterocyclyl, aryl, and heteroaryl of R³ is independently unsubstituted or substituted with one to three R¹⁰;
each R¹⁰ is independently —OR¹², —N(R¹²)₂, —S(O)₂R¹³, —OC(O)CHR¹²N(R¹²)₂, or C₁-C₆alkyl, wherein the C₁-C₆alkyl, of R¹⁰ is independently unsubstituted or substituted with one to three Ru;
each R¹¹ is independently halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl, wherein the heterocyclyl is independently unsubstituted or substituted with one to three R¹⁰;
each R¹² is independently hydrogen, C₁-C₆alkyl, or C₃-C₁₀cycloalkyl; and
each R¹³ is independently C₁-C₆alkyl, or C₃-C₁₀cycloalkyl.

In some embodiments, for a compound of any one of Formulas II, IIA, IIB, IIC, IID, IIE, IIF, IIG, or IIH, ring A is bicyclo[1.1.1]pentan-1-yl, phenyl, piperidinyl, pyrazolyl, pyridyl, or quinolinyl, each of which is unsubstituted or substituted by one, two, or three R³. In certain embodiments, ring A is bicyclo[1.1.1]pentan-1-yl, phenyl, piperidinyl, pyrazolyl, pyridyl, or quinolinyl, each of which is substituted by one, two or three R³. In certain embodiments, ring A is bicyclo[1.1.1]pentan-1-yl, phenyl, piperidinyl, pyrazolyl, pyridyl, or quinolinyl, each of which is substituted by two or three R³.

In some embodiments, for a compound of any one of Formulas II, IIA, IIB, IIC, IID, IIE, IIF, IIG, or IIH, ring A is aryl or heteroaryl, each of which is substituted by two or three R³. In certain embodiments, ring A is aryl or heteroaryl, each of which is substituted by two or three R³, wherein at least one R³ is halo.

In some embodiments, for a compound of any one of Formulas II, IIA, IIB, IIC, IID, IIE, IIF, IIG, or IIH, ring A is cyclohexyl. In certain embodiments, ring A is C₄-C₁₀cycloalkyl. In certain embodiments, ring A is a C₄-C₇cycloalkyl. In certain embodiments, ring A is bicyclo[1.1.1]pentanyl. In certain embodiments, ring A is selected from cyclobutyl, cyclopentyl, cyclohexyl, and cycloheptyl.

In some embodiments, for a compound of any one of Formulas II, IIA, IIB, IIC, IID, IIE, IIF, IIG, or IIH, ring A, or the moiety

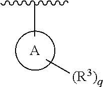

is:

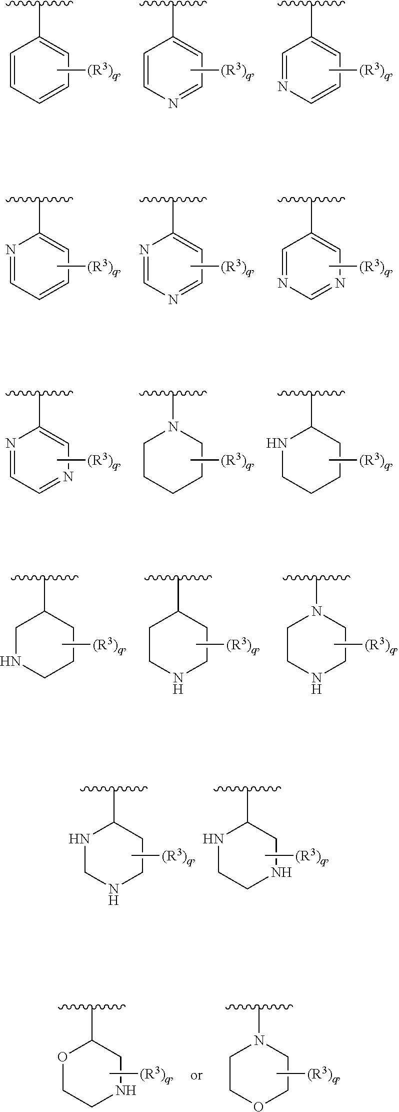

where q and each R³ is independently as defined herein. In some embodiments, for a compound of any one of II, IIA, IIB, IIC, IID, IIE, IIF, IIG, or IIH, ring A, or the moiety

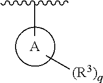

is:

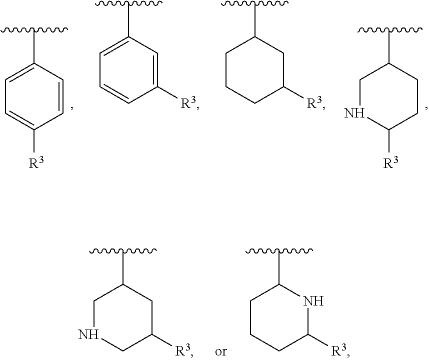

where R³ is independently as defined herein.

In some embodiments, for a compound of any one of Formulas II, IIA, IIB, IIC, IID, IIE, IIF, IIG, or IIH, ring A is a bridged bicyclic ring selected from:

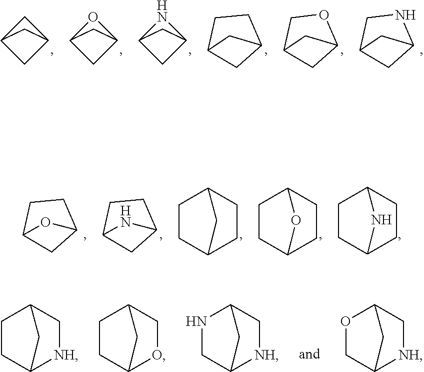

and wherein each is substituted with one to three R³. In certain embodiments, ring A is a bridged bicyclic ring selected from:

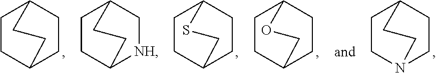

wherein each R³ is attached to a carbon atom on the bridged bicyclic ring.

In some embodiments, for a compound of any one of Formulas II, IIA, IIB, IIC, IID, IIE, IIF, IIG, or IIH, ring A, or the moiety

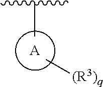

is:

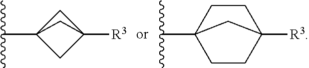

In some embodiments, the compound is a compound of Formula IIJ:

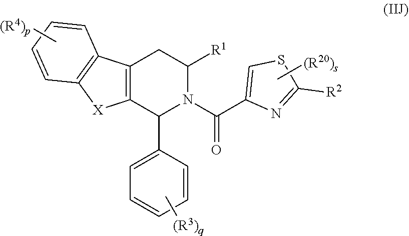

or a tautomer, stereoisomer, mixture of stereoisomers, isotopically enriched analog, or pharmaceutically acceptable salt thereof, where each of X, R¹, R², R³, R⁴, R²⁰, p, q, and s are as defined herein.

In some embodiments, the compound is a compound of Formula IIK:

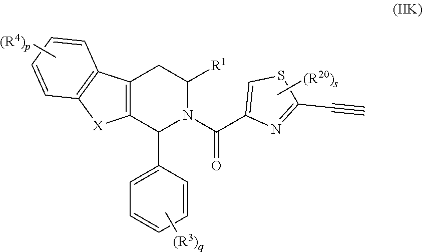

or a tautomer, stereoisomer, mixture of stereoisomers, isotopically enriched analog, or pharmaceutically acceptable salt thereof, where each of X, R¹, R³, R⁴, R²⁰, p, q, and s are as defined herein.

In some embodiments, the compound is a compound of Formula IIL:

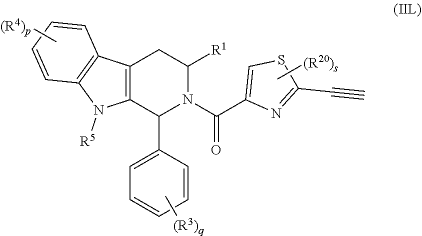

or a tautomer, stereoisomer, mixture of stereoisomers, isotopically enriched analog, or pharmaceutically acceptable salt thereof, where each of R¹, R³, R⁴, R⁵, R²⁰, p, q, and s are as defined herein.

In some embodiments, the compound is a compound of Formula IIM:

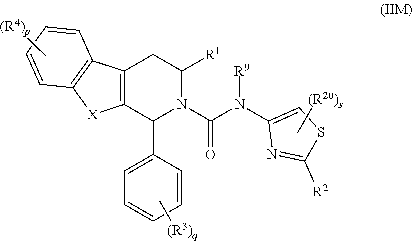

or a tautomer, stereoisomer, mixture of stereoisomers, isotopically enriched analog, or pharmaceutically acceptable salt thereof, wherein each of X, R¹, R², R³, R⁴, R⁹, R²⁰, p, q, and s are independently as defined herein.

In some embodiments, the compound is a compound of Formula IIN:

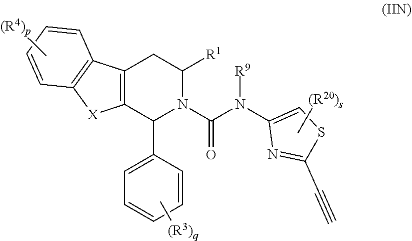

or a tautomer, stereoisomer, mixture of stereoisomers, isotopically enriched analog, or pharmaceutically acceptable salt thereof, wherein each of X, R¹, R³, R⁴, R⁹, R²⁰, p, q, and s are independently as defined herein.

In some embodiments, the compound is a compound of Formula IIP:

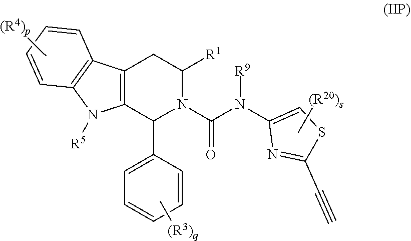

or a tautomer, stereoisomer, mixture of stereoisomers, isotopically enriched analog, or pharmaceutically acceptable salt thereof, wherein each of R¹, R³, R⁴, R⁵, R⁹, R²⁰, p, q, and s are independently as defined herein.

In some embodiments, for a compound of Formula IIL or IIP, R⁵ is H. In some embodiments, R⁵ is C₁-C₆alkyl.

In some embodiments, for a compound of any one of Formulas IIM, IIN, or IIP, R⁹ is H. In some embodiments, R⁹ is C₁-C₆alkyl.

In some embodiments, for a compound of any one of Formulas II, IIA, IIB, IIC, IID, IIE, IIF, IIG, IIH, IIJ, IIK, IIL, IIM, IIN, or IIP, q is 0. In some embodiments, q is 1, 2, or 3. In some embodiments, q is 1. In some embodiments, q is 2. In some embodiments, q is 3. In some embodiments, q is 1, 2, or 3 and at least one R³ is halo, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)R⁸, —C(O)R⁶, or —OC(O)CHR⁸N(R¹²)₂. In some embodiments, q is 1, 2, or 3 and each R³ is independently halo, —OR⁸, —NHR⁸, —N(R⁸)₂, —S(O)₂N(R⁷)₂, —S(O)N(R⁷)₂, —C(O)N(R⁷)₂, —NR¹²C(O)R⁵, —NR¹²C(O)OR⁸, —OC(O)N(R⁷)₂, or —OC(O)CHR⁸N(R¹²)₂. In some embodiments, q is 1, 2, or 3 and each R³ is independently halo, —OR⁸, —NHR⁸, —N(R⁸)₂, —S(O)₂N(R⁷)₂, or —C(O)N(R⁷)₂. In some embodiments, q is 1, and R³ is halo, —OR⁸, —NHR⁸, —N(R⁸)₂, —S(O)₂N(R⁷)₂, or —C(O)N(R⁷)₂. In some embodiments, q is 1, and R³ is halo. In some embodiments, q is 1, and R³ is —OR⁸. In some embodiments, q is 1, and R³ is —NHR⁸. In some embodiments, q is 1, and R³ is —N(R⁸)₂. In some embodiments, q is 1, and R³ is —S(O)₂N(R⁷)₂. In some embodiments, q is 1, and R³ is —C(O)N(R⁷)₂. In some embodiments, q is 2 or 3; at least one R³ is halo (e.g., F); and at least one R³ is selected from —OR⁸, —NHR⁸, —N(R⁸)₂, —S(O)₂N(R⁷)₂, and —C(O)N(R⁷)₂. In some embodiments, q is 1, 2, or 3 and at least one R³ is selected from —S(O)₂N(R⁷)₂ and —C(O)N(R⁷)₂, wherein the two R⁷ groups, together with the nitrogen atom to which they are attached, form a 4 to 7 membered heterocyclyl, wherein the heterocyclyl formed by the two R⁷ groups is unsubstituted or substituted with a 4- to 6-membered heterocyclyl or —N(C₁-C₆alkyl)₂, wherein the 4- to 6-membered heterocyclyl when containing 2 or more N atoms is unsubstituted or substituted with an N-protecting group. In certain embodiments, the 4 to 7 membered heterocyclyl is selected from azetidinyl, pyrrolidinyl, piperidinyl, pyrazolidinyl, isoxazolidinyl, oxazolidinyl, thiazolidinyl, imidazolidinyl, piperazinyl, morpholinyl, thiomorpholinyl, 1,3-oxazinanyl, 1,3-thiazinanyl, dihydropyridinyl, tetrahydropyranyl, 1,3-tetrahydropyrimidinyl, dihydropyrimidinyl, azepanyl and 1,4-diazepanyl. In certain embodiments, the 4- to 6-membered heterocyclyl, when present as a substituent, is selected from azetidinyl, oxetanyl, thietanyl, piperidinyl, 1,2,3,6-tetrahydropyridinyl, pyranyl, dioxanyl, 1,3-dioxolanyl, dihydropyranyl, dihydrothienyl, dihydrofuranyl, imidazolinyl, pyrrolidinyl, piperidinyl, pyrazolidinyl, isoxazolidinyl, oxazolidinyl, thiazolidinyl, piperazinyl, morpholinyl, thiomorpholinyl, 1,3-oxazinanyl, 1,3-thiazinanyl, dihydropyridinyl, 1,3-tetrahydropyrimidinyl, and dihydropyrimidinyl. In certain embodiments, the N-protecting group when present is t-Boc.

In some embodiments, for a compound of any one of Formulas II, IIA, IIB, IIC, IID, IIE, IIF, IIG, IIH, IIJ, IIK, IIL, IIM, IIN, or IIP, q is 1, 2, or 3 and at least one R³ is halo, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)R⁸, —C(O)R⁶, or —OC(O)CHR⁸N(R¹²)₂. In certain embodiments, q is 1, 2, or 3 and at least one R³ is halo. In some embodiments, q is 1, 2, or 3 and at least one R³ is —NHR⁸. In certain embodiments, q is 1, 2, or 3 and at least one R³ is —N(R⁸)₂. In certain embodiments, q is 2, and one R³ is halo and the other R³ is —N(R⁸)₂. In certain embodiments, q is 3, and two R³ are independently halo and one R³ is —N(R⁸)₂. In some embodiments, q is 1, 2, or 3 and at least one R³ is —C(O)OR⁶ or —C(O)R⁶. In certain embodiments, q is 1, 2, or 3 and at least one R³ is —S(O)₂N(R⁷)₂, —S(O)N(R⁷)₂, or —C(O)N(R⁷)₂. In certain embodiments, q is 1, 2, or 3 and at least one R³ is —S(O)₂R⁸, —S(O)R⁸, —NR¹²C(O)R⁸, —NR¹²C(O)OR⁸, —OC(O)R⁸, or —OC(O)CHR⁸N(R¹²)₂. In some embodiments, q is 1, 2, or 3 and each R³ is independently halo, —CN, —OR⁸, —NHR⁸, —S(O)₂R⁸, —S(O)₂N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)R⁸, —OC(O)CHR⁸N(R¹²)₂, C₁-C₆alkyl, C₃-C₁₀cycloalkyl, heterocyclyl, heteroaryl, or —C₁-C₆alkylheterocyclyl; wherein each C₁-C₆alkyl, C₃-C₁₀cycloalkyl, heterocyclyl, heteroaryl, or —C₁-C₆alkylheterocyclyl of R³ is independently unsubstituted or substituted with one to three R¹⁰. In some embodiments, q is 1, 2, or 3 and each R³ is independently halo, —CN, —OR⁸, —NHR⁸, —S(O)₂R⁸, —S(O)₂N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)R⁸, —OC(O)CHR⁸N(R¹²)₂, C₁-C₆alkyl, C₃-C₁₀cycloalkyl, heterocyclyl, heteroaryl, or —C₁-C₆alkylheterocyclyl; wherein each C₁-C₆alkyl, C₃-C₁₀cycloalkyl, heterocyclyl, heteroaryl, or —C₁-C₆alkylheterocyclyl is independently unsubstituted or substituted with one to three substituents independently selected from —OR¹², —N(R¹²)₂, —S(O)₂R¹³, —OC(O)CHR¹²N(R¹²)₂, and C₁-C₆alkyl optionally substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl; wherein each R¹² is independently hydrogen, C₁-C₆alkyl or C₃-C₁₀cycloalkyl; and each R¹³ is independently C₁-C₆alkyl or C₃-C₁₀cycloalkyl.

In some embodiments, for a compound of any one of Formulas II, IIA, IIB, IIC, IID, IIE, IIF, IIG, IIH, III, IIK, IIL, IIM, IIN, or IIP, q is 1, 2, or 3 and each R³ is independently —NH₂, fluoro, methyl, pyridine-4-carboxamido, pyridin-3-amino, pentyloxycarbonylamino, N-(3-aminobicyclo[1.1.1]pentan-1-yl)amino, morpholin-4-yl, methoxycarbonyl, dimethylcarbamoyl, cyclopropylcarbamoyl, cyclohexyl, cyclobutylcarbamoyl, cyclobutylaminosulfonyl, adamantylamino, (adamantan-1-ylamino)methyl, 3-methyl-1,2,4-oxadiazol-5-yl, 2-methylpyridine-4-carboxamido, (bicyclo[1.1.1]pentan-1-ylamino)methyl, (adamantan-1-yl)carbamoyl, or (2-methoxyethyl)carbamoyl.

In some embodiments, for a compound of any one of Formulas II, IIA, IIB, IIC, IID, IIE, IIF, IIG, IIH, IIJ, IIK, IIL, IIM, IIN, or IIP, q is 1, and R³ is —NH₂, fluoro, methyl, pyridine-4-carboxamido, pyridin-3-amino, pentyloxycarbonylamino, N-(3-aminobicyclo[1.1.1]pentan-1-yl)amino, morpholin-4-yl, methoxycarbonyl, dimethylcarbamoyl, cyclopropylcarbamoyl, cyclohexyl, cyclobutylcarbamoyl, cyclobutylaminosulfonyl, adamantylamino, (adamantan-1-ylamino)methyl, 3-methyl-1,2,4-oxadiazol-5-yl, 2-methylpyridine-4-carboxamido, (bicyclo[1.1.1]pentan-1-ylamino)methyl, (adamantan-1-yl)carbamoyl, or (2-methoxyethyl)carbamoyl.

In some embodiments, for a compound of any one of Formulas II, IIA, IIB, IIC, IID, IIE, IIF, IIG, IIH, IIJ, IIK, IIL, IIM, IIN, or IIP, R¹ is C₁-C₆alkyl, C₂-C₆alkenyl, C₂-C₆alkynyl, C₁-C₆haloalkyl, C₃-C₁₀cycloalkyl, —CN, —C(O)OR⁶, —C(O)N(R⁷)₂, —NH₂, —NHR⁸, —N(R⁸)₂, —OH, —OR⁸, —C₁-C₆alkyl-OH, or —C₁-C₆alkyl-OR⁸. In certain embodiments, R¹ is C₁-C₆alkyl, C₂-C₆alkenyl, C₂-C₆alkynyl, C₁-C₆haloalkyl, —C(O)OR⁶, —C(O)N(R⁷)₂, —NH₂, —NHR⁸, —N(R⁸)₂, —OH, —OR⁸, —C₁-C₆alkyl-OH or —C₁-C₆alkyl-OR⁸. In certain embodiments, R¹ is C₁-C₆alkyl, C₂-C₆alkenyl, C₂-C₆alkynyl, C₁-C₆haloalkyl, —CN, C₃-C₁₀cycloalkyl, —NH₂, —NHR⁸, —N(R⁸)₂, —OH, —OR⁸, —C₁-C₆alkyl-OH or —C₁-C₆alkyl-OR⁸. In certain embodiments, R¹ is C₁-C₆alkyl, C₂-C₆alkenyl, C₂-C₆alkynyl, C₁-C₆haloalkyl, —NH₂, —NHR⁸, —N(R⁸)₂, —OH, —OR⁸, —C₁-C₆alkyl-OH or —C₁-C₆alkyl-OR⁸. In certain embodiments, R¹ is —C(O)OR⁶ or —C(O)N(R⁷)₂. In certain embodiments, R¹ is C₁-C₆alkyl. In certain embodiments, R¹ is C₂-C₆alkyl. In certain embodiments, R¹ is C₃-C₆alkyl. In certain embodiments, R¹ is C₅-C₆alkyl. In certain embodiments, R¹ is C₂-C₃alkyl. In certain embodiments, R¹ is C₄-C₆alkyl. In certain embodiments, R¹ is methyl. In certain embodiments, R¹ is n-butyl. In certain embodiments, R¹ is C₃-C₁₀cycloalkyl.

In some embodiments, for a compound of any one of Formulas II, IIA, IIB, IIC, IID, IIE, IIF, IIG, IIH, III, IIK, IIL, IIM, IIN, or IIP, p is 0. In some embodiments, p is 1, 2, or 3. In some embodiments, p is 1. In some embodiments, p is 2. In some embodiments, p is 3. In some embodiments, p is 1, 2, or 3 and each R⁴ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆alkyl, or C₂-C₆alkenyl, wherein each C₁-C₆alkyl, C₂-C₆alkenyl is independently unsubstituted or substituted with one to three R¹⁰. In some embodiments, p is 1, 2, or 3 and each R⁴ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆alkyl, or C₂-C₆alkenyl, wherein each C₁-C₆alkyl, C₂-C₆alkenyl is independently unsubstituted or substituted with one to three R¹⁰. In some embodiments, p is 1, 2, or 3 and each R⁴ is independently halo, —CN, —OH, —OR⁸, —NH₂, —NHR⁸, —N(R′)₂, —S(O)₂R⁸, —S(O)R⁵, —S(O)₂N(R⁷)₂, —S(O)N(R⁷)₂, —NO₂, —Si(R's)₃, —C(O)OR⁶, —C(O)N(R⁷)₂, —NR¹²C(O)R⁸, —OC(O)R⁸, —C(O)R⁶, C₁-C₆alkyl, C₂-C₆alkenyl, C₂-C₆alkynyl, or C₃-C₁₀cycloalkyl; wherein each C₁-C₆alkyl, C₂-C₆alkenyl, C₂-C₆alkynyl, or C₃-C₁₀cycloalkyl of R⁴ is independently optionally substituted with one to three R¹⁰. In some embodiments, p is 1, 2, or 3 and each R⁴ is independently halo, —CN, —OR⁷, C₁-C₆alkyl, C₂-C₆alkynyl, or C₃-C₁₀cycloalkyl; wherein each C₁-C₆alkyl, C₂-C₆alkynyl, or C₃-C₁₀cycloalkyl of R⁴ is independently unsubstituted or substituted with one to three R¹⁰. In certain embodiments, p is 1, 2, or 3 and each R⁴ is independently halo, —CN, —OH, C₁-C₆alkyl, C₂-C₆alkynyl, or C₃-C₁₀cycloalkyl. In certain embodiments, p is 1, 2, or 3 and each R⁴ is independently halo, —CN, —OH, —OR⁸, C₁-C₆alkyl, or C₂-C₆alkynyl; wherein the C₁-C₆alkyl of R⁴ is unsubstituted or substituted with one to three R¹⁰. In some embodiments, p is 1, 2, or 3 and each R⁴ is independently halo, —CN, —OH, —OR⁸, C₁-C₆alkyl, C₂-C₆alkynyl; wherein the C₁-C₆alkyl of R⁴ is unsubstituted or substituted with one to three substituents independently selected from —OR¹², —N(R¹²)₂, —S(O)₂R¹³, —OC(O)CHR¹²N(R¹²)₂, and C₁-C₆alkyl optionally substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl; wherein each R¹² is independently hydrogen, C₁-C₆alkyl or C₃-C₁₀cycloalkyl; and each R¹³ is independently C₁-C₆alkyl or C₃-C₁₀cycloalkyl. In some embodiments, p is 1, 2, or 3 and at least one R⁴ is C₁-C₆alkyl substituted with one to three R¹⁰, wherein the one to three R¹⁰ are independently —OP(O)(OR¹²)₂, —OC(O)CHR¹²N(R¹²)₂, —OR¹², or —C(O)OR¹². In some embodiments, p is 1, 2, or 3 and at least one R⁴ is —OR⁸. In some embodiments, p is 1, 2, or 3 and at least one R⁴ is —NR¹²C(O)R⁸, wherein R¹ is a heterocyclyl.

In some embodiments, for a compound of any one of Formulas II, IIA, IIB, IIC, IID, IIE, IIF, IIG, IIH, IIJ, IIK, IIL, IIM, IIN, or IIP, each R⁶ is independently hydrogen, C₁-C₆alkyl, C₂-C₆alkenyl, or —C₁-C₆alkylC₃-C₁₀cycloalkyl; wherein each R⁶ is independently unsubstituted or substituted with one to three R¹¹. In certain embodiments, each R⁶ is independently hydrogen, C₁-C₆alkyl, C₂-C₆alkenyl, or —C₁-C₆alkylC₃-C₁₀cycloalkyl; wherein each R⁶ is independently unsubstituted or substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl; wherein each R² is independently hydrogen, C₁-C₆alkyl or C₃-C₁₀cycloalkyl.

In some embodiments, for a compound of any one of Formulas II, IIA, IIB, IIC, IID, IIE, IIF, IIG, IIH, IIJ, IIK, IIL, IIM, IIN, or IIP, each R⁷ is independently hydrogen, C₁-C₆alkyl, C₃-C₁₀cycloalkyl, heterocyclyl, heteroaryl, —C₁-C₆alkylC₃-C₆cycloalkyl, —C₁-C₆alkylheterocyclyl, or two R⁷, together with the nitrogen atom to which they are attached, form a 4 to 7 membered heterocyclyl; wherein each R⁷ or ring formed thereby is independently unsubstituted or substituted with one to three R¹¹. In certain embodiments, each R⁷ is independently hydrogen, C₁-C₆alkyl, C₃-C₁₀cycloalkyl, heterocyclyl, heteroaryl, —C₁-C₆alkylC₃-C₆cycloalkyl, —C₁-C₆alkylheterocyclyl, or two R⁷, together with the nitrogen atom to which they are attached, form a 4 to 7 membered heterocyclyl; wherein each R⁷ or ring formed thereby is independently unsubstituted or substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl; wherein each R¹² is independently hydrogen, C₁-C₆alkyl or C₃-C₁₀cycloalkyl.

In some embodiments, for a compound of any one of Formulas II, IIA, IIB, IIC, IID, IIE, IIF, IIG, IIH, IIJ, IIK, IIL, IIM, IIN, or IIP, each R¹ is independently C₁-C₆alkyl, C₂-C₆alkynyl, C₃-C₁₀cycloalkyl, —C₁-C₆alkylC₃-C₁₀cycloalkyl, or —C₁-C₆alkylaryl; wherein each R¹ is independently unsubstituted or substituted with one to three R¹¹. In certain embodiments, each R¹ is independently C₁-C₆alkyl, C₂-C₆alkynyl, C₃-C₁₀cycloalkyl, —C₁-C₆alkylC₃-C₁₀cycloalkyl, or —C₁-C₆alkylaryl; wherein each R¹ is independently further substituted with one to three halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl; wherein each R¹² is independently hydrogen, C₁-C₆alkyl or C₃-C₁₀cycloalkyl.

In some embodiments, for a compound of any one of Formulas II, IIA, IIB, IIC, IID, IIE, IIF, IIG, IIH, IIJ, IIK, IIL, IIM, IIN, or IIP, each R¹⁰ is independently —OR¹², —N(R¹²)₂, —S(O)₂R¹³, —OC(O)CHR¹²N(R¹²)₂, or C₁-C₆alkyl, wherein the C₁-C₆alkyl, of R^{1′} is independently unsubstituted or substituted with one to three R¹¹; each R¹¹ is independently halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl; each R¹² is independently hydrogen, C₁-C₆alkyl, or C₃-C₁₀cycloalkyl; and each R¹³ is independently C₁-C₆alkyl or C₃-C₁₀cycloalkyl.

In some embodiments, for a compound of any one of Formulas II, IIA, IIB, IIC, IID, IIE, IIF, IIG, IIH, IIJ, IIK, IIL, IIM, IIN, or IIP, each R¹⁰ is independently adamantyl or adamantylC₁-C₆aliphatic-. In some embodiments, the adamantyl is selected from the following:

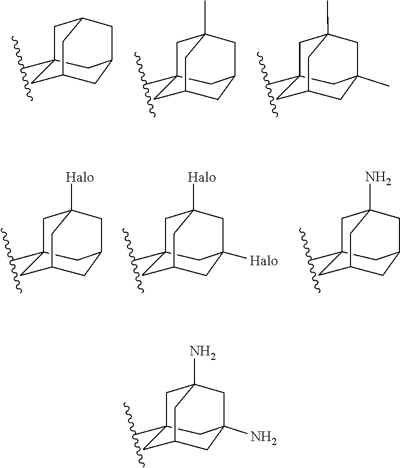

In some embodiments, for a compound of any one of Formulas II, IIA, IIB, IIC, IID, IIE, IIF, IG, or IIH:
ring A is C₄-C₁₀cycloalkyl, heterocyclyl, aryl, or heteroaryl;
X is —NR⁵— or —S—;
L, if present, is absent or —NR⁹.
p is 0, 1, 2 or 3;
q is 0, 1, 2 or 3;
s is 0, 1, 2, or 3;
R¹ is C₁-C₆alkyl, C₁-C₆haloalkyl, C₃-C₁₀cycloalkyl, —CN, —C(O)OR⁶, —C(O)N(R⁷)₂, —C₁-C₆alkyl-OH or —C₁-C₆alkyl-OR⁸;
each R³ is independently halo, —CN, —OR⁸, —NHR⁸, —S(O)₂R⁸, —S(O)₂N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)R⁸, —OC(O)CHR⁸N(R¹²)₂, C₁-C₆alkyl, C₃-C₁₀cycloalkyl, heterocyclyl, heteroaryl, or —C₁-C₆alkylheterocyclyl; wherein each C₁-C₆alkyl, C₃-C₁₀cycloalkyl, heterocyclyl, heteroaryl, or —C₁-C₆alkylheterocyclyl of R³ is independently optionally substituted with one to three R¹⁰.
each R⁴ is independently halo, —CN, —OH, —OR⁸, C₁-C₆alkyl, or C₂-C₆alkynyl; wherein the C₁-C₆alkyl of R⁴ is optionally independently optionally substituted with one to three R¹⁰.
R⁵ is hydrogen or C₁-C₆alkyl;
each R⁶ is independently hydrogen, C₁-C₆alkyl, C₂-C₆alkenyl, or —C₁-C₆alkylC₃-C₁₀cycloalkyl; wherein each R⁶ is independently further substituted with one to three R¹¹;
each R⁷ is independently hydrogen, C₁-C₆alkyl, C₃-C₁₀cycloalkyl, heterocyclyl, heteroaryl, —C₁-C₆alkylC₃-C₆cycloalkyl, —C₁-C₆alkylheterocyclyl, or two R⁷ together with the nitrogen atom to which they are attached, form a 4 to 7 membered heterocyclyl; wherein each R⁷ or ring formed thereby is independently further substituted with one to three R¹¹;
each R⁸ is independently C₁-C₆alkyl, C₂-C₆alkynyl, C₃-C₁₀cycloalkyl, —C₁-C₆alkylC₃-C₁₀cycloalkyl, or —C₁-C₆alkylaryl; wherein each R⁸ is independently further substituted with one to three R¹¹;
R⁹, if present, is hydrogen or C₁-C₆alkyl;
each R¹⁰ is independently —OR¹², —N(R¹²)₂, —S(O)₂R¹³, —OC(O)CHR¹²N(R¹²)₂, or C₁-C₆alkyl, wherein the C₁-C₆alkyl, of R¹⁰ is optionally independently substituted with one to three R¹¹;
each R¹¹ is independently halo, —OR¹², —N(R¹²)₂, —Si(R¹²)₃, —C(O)OR¹², —NR¹²C(O)OR¹², —OC(O)CHR¹²N(R¹²)₂, C₁-C₆alkyl, or heterocyclyl;
each R¹² is independently hydrogen, C₁-C₆alkyl or C₃-C₁₀cycloalkyl;
each R¹³ is independently C₁-C₆alkyl or C₃-C₁₀cycloalkyl; and
each R²⁰ is independently halogen, C₁-C₆alkyl, or C₂-C₆alkenyl.

In certain embodiments, each R¹⁵ is independently C₁-C₆alkyl.

Also provided herein are embodiments wherein any embodiment described herein may be combined with any one or more of these embodiments, provided the combination is not mutually exclusive. As used herein, two embodiments are “mutually exclusive” when one is defined to be something which is different than the other. For example, an embodiment wherein two groups combine to form a ring is mutually exclusive with an embodiment in which one group is ethyl and the other group is hydrogen. Similarly, an embodiment wherein one group is CH₂ is mutually exclusive with an embodiment wherein the same group is NH.

In some embodiments of any of the preceding aspects, the compound is a compound, or a tautomer, stereoisomer, mixture of stereoisomers, isotopically enriched analog, ester, prodrug, zwitterionic form, or pharmaceutically acceptable salt of a compound included in Table 1. Also provided herein is a compound selected from Table 1 or any of the Examples provided herein, or a tautomer, stereoisomer, mixture of stereoisomers, isotopically enriched analog, ester, prodrug, zwitterionic form, or pharmaceutically acceptable salt thereof.

TABLE 1
Selected Compounds of the Present Disclosure.

| Compound | Structure | Characterization data |
|---|---|---|
| 1 | 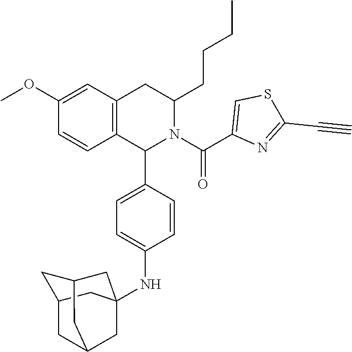 [SMILES: C#Cc1nc(C(=O)N2C(CCCC)Cc3cc(OC)ccc3C2c2ccc(NC34CC5CC(CC(C5)C3)C4)cc2)cs1] | |
| 1a | 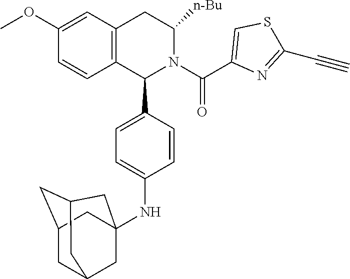 | LC-MS (m/z): 580.6 [M + H]⁺; ¹H NMR (400 MHz, CHLOROFORM-d) δ 8.00 (br s, 0.39 H), 7.45 (br s, 0.49 H), 7.19 (br s, 1H), 7.09-6.96 (m, 1H), 6.86- 6.73 (m, 2H), 6.68 (d, J = 2.3 Hz, 1H), 6.66-6.46 (m, 3H), 5.40 (br s, 0.46 H), 4.84 (br s, 0.52 H), 3.79 (s, 3H), 3.49 (br s, 1H), 3.23-3.00 (m, 1H), 2.85-2.65 (m, 1H), 2.06 (br s, 3H), 1.78 (br s, 6H), 1.70-1.54 (m, 6H), 1.27-1.19 (m, 2H), 1.17-0.89 (m, 4H), 0.88-0.66 (m, 3H). |
| 2 | 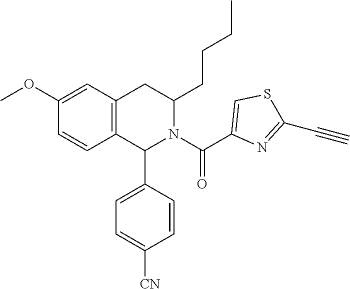 [SMILES: C#Cc1nc(C(=O)N2C(CCCC)Cc3cc(OC)ccc3C2c2ccc(C#N)cc2)cs1] | |
| 2a | 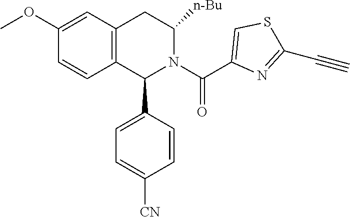 | LC-MS (m/z): 456.0 [M + Na]⁺; ¹H NMR (400 MHz, CHLOROFORM-d) δ 8.06- 7.68 (m, 1H), 7.58-7.38 (m, 3H), 7.23 (br d, J = 8.5 Hz, 1H), 7.09 (br s, 1H), 6.82-6.76 (m, 1H), 6.70 (br s, 1H), 6.40 (s, 1H), 5.50 (br s, 1H), 3.79 (s, 3H), 3.60-3.47 (m, 1H), 3.23 (br dd, J = 3.9, 15.3 Hz, 1H), 3.01-2.70 (m, 1H), 1.37- 0.96 (m, 7H), 0.90-0.64 (m, 3H). |
| 3 | 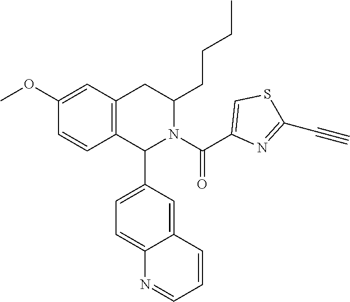 [SMILES: C#Cc1nc(C(=O)N2C(CCCC)Cc3cc(OC)ccc3C2c2ccc3ncccc3c2)cs1] | |
| 3a | 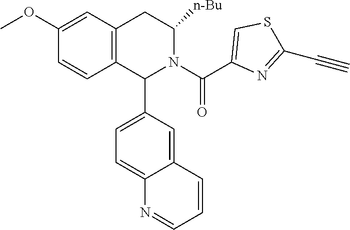 | LC-MS (m/z): 482.1 [M + H]⁺; ¹H NMR (400 MHz, DMSO-d6) δ 8.91 (br d, J = 4.4 Hz, 1H), 8.53 (br s, 1H), 8.25-7.74 (m, 4H), 7.63 (br d, J = 4.8 Hz, 1H), 7.46 (br d, J = 8.4 Hz, 1H), 6.91- 6.75 (m, 2H), 6.55 (br s, 1H), 5.04 (br s, 1H), 4.85 (br s, 1H), 3.73 (s, 3H), 2.90 (br d, J = 15.6 Hz, 1H), 1.54-0.96 (m, 6H), 0.74 (br s, 3H). |
| 4 | 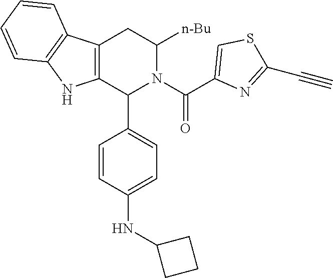 | |
| 4a | 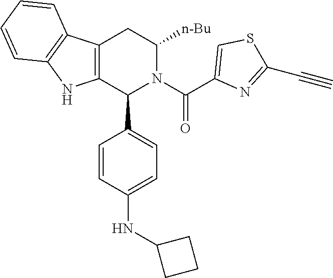 | LC-MS (m/z): 509.1 [M + H]⁺; ¹H NMR (400 MHz, DMSO-d₆) δ 10.56 (br s, 1H), 8.01 (s, 1H), 7.43 (d, J = 7.6 Hz, 1H), 7.23 (d, J = 7.6 Hz, 1H), 7.08-6.91 (m, 4H), 6.39 (d, J = 8.8 Hz, 2H), 6.19 (s, 1H), 5.52 (br s, 1H), 4.84 (d, J = 2.4 Hz, 1H), 4.34 (br s, 1H), 3.78 (sxt, J = 7.2 Hz, 1H), 3.06- 2.93 (m, 2H), 2.35-2.23 (m, 2H), 1.87- 1.44 (m, 6H), 1.22-0.97 (m, 4H), 0.78- 0.67 (m, 3H). |
| 5 | 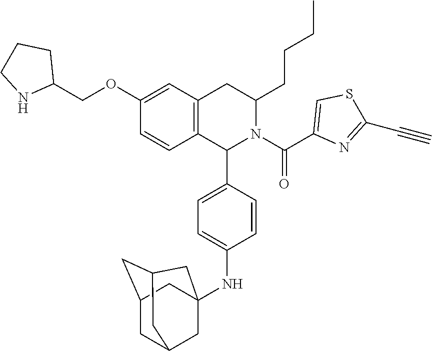 [SMILES: C#Cc1nc(C(=O)N2C(CCCC)Cc3cc(OCC4CCCN4)ccc3C2c2ccc(NC34CC5CC(CC(C5)C3)C4)cc2)cs1] | |
| 5a | 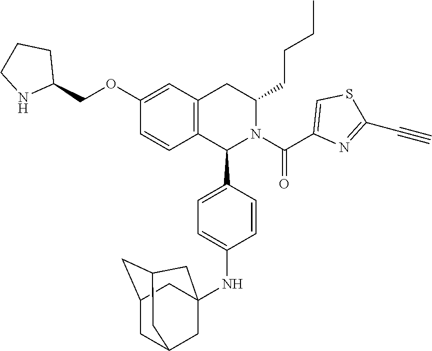 [SMILES: C#Cc1nc(C(=O)N2[C@@H](CCCC)Cc3cc(OC[C@@H]4CCCN4)ccc3[C@@H]2c2ccc(NC34CC5CC(CC(C5)C3)C4)cc2)cs1] | LC-MS (m/z): 671.3 [M + Na]⁺; ¹H NMR (400 MHz, METHANOL-d₄) δ 8.17 (br s, 1H), 7.93-7.78 (m, 1H), 7.66 (br s, 1H), 7.44 (br d, J = 8.8 Hz, 2H), 7.27 (br s, 2H), 7.01-6.80 (m, 2H), 6.40 (br s, 1H), 5.31-4.95 (m, 2H), 4.42-4.31 (m, 2H), 4.16-3.97 (m, 2H), 3.45-3.33 (m, 3H), 3.16 (br d, J = 15.6 Hz, 1H), 2.93 (br d, J = 14.4 Hz, 1H), 2.33-2.02 (m, 6H), 1.99-1.58 (m, 13H), 1.43-0.64 (m, 9H). |
| 5b | 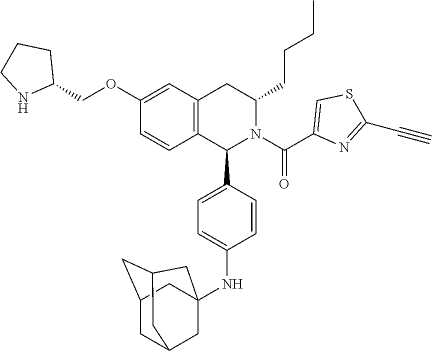 | LC-MS (m/z): 566.3 [M + H]⁺; ¹H NMR (400 MHz, DMSO-d₆) δ 10.56 (br s, 1H), 8.01 (s, 1H), 7.43 (d, J = 7.6 Hz, 1H), 7.23 (d, J = 7.6 Hz, 1H), 7.08-6.91 (m, 4H), 6.39 (d, J = 8.8 Hz, 2H), 6.19 (s, 1H), 5.52 (br s, 1H), 4.84 (d, J = 2.4 Hz, 1H), 4.34 (br s, 1H), 3.78 (sxt, J = 7.2 Hz, 1H), 3.06-2.93 (m, 2H), 2.35-2.23 (m, 2H), 1.87-1.44 (m, 6H), 1.22-0.97 (m, 4H), 0.78-0.67 (m, 3H). |
| 6 | 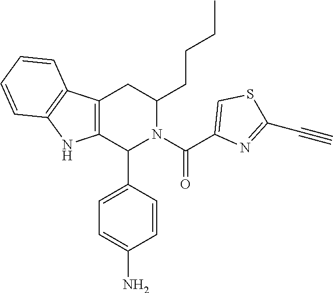 [SMILES: C#Cc1nc(C(=O)N2C(CCCC)Cc3c([nH]c4ccccc34)C2c2ccc(N)cc2)cs1] | |
| 6a | 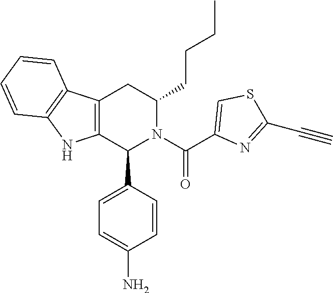 | LC-MS (m/z): 509.1 [M + H]⁺; ¹H NMR (400 MHz, DMSO-d₆) δ 10.56 (br s, 1H), 8.01 (s, 1H), 7.43 (d, J = 7.6 Hz, 1H), 7.23 (d, J = 7.6 Hz, 1H), 7.08- 6.91 (m, 4H), 6.39 (d, J = 8.8 Hz, 2H), 6.19 (s, 1H), 5.52 (br s, 1H), 4.84 (d, J = 2.4 Hz, 1H), 4.34 (br s, 1H), 3.78 (sxt, J = 7.2 Hz, 1H), 3.06-2.93 (m, 2H), 2.35- 2.23 (m, 2H), 1.87-1.44 (m, 6H), 1.22- 0.97 (m, 4H), 0.78-0.67 (m, 3H). |
| 7 | 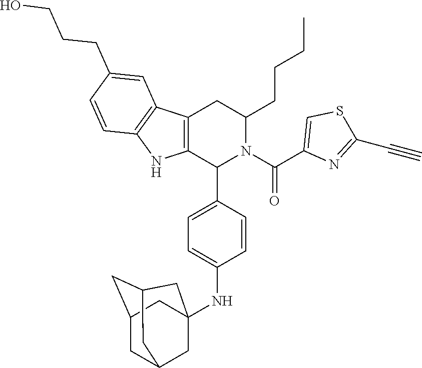 [SMILES: C#Cc1nc(C(=O)N2C(CCCC)Cc3c([nH]c4ccc(CCCO)cc34)C2c2ccc(NC34CC5CC(CC(C5)C3)C4)cc2)cs1] | |
| 7a | 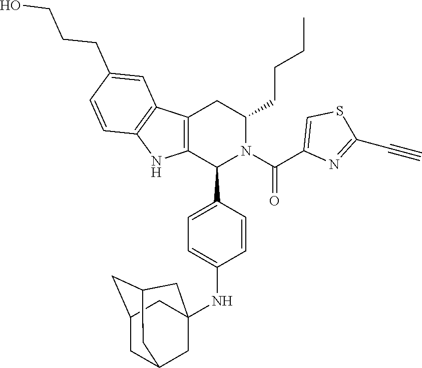 [SMILES: C#Cc1nc(C(=O)N2[C@@H](CCCC)Cc3c([nH]c4ccc(CCCO)cc34)[C@@H]2c2ccc(NC34CC5CC(CC(C5)C3)C4)cc2)cs1] | LC-MS (m/z): 647.3 [M + H]⁺; ¹H NMR (400 MHz, DMSO-d₆) δ 10.45 (br s, 1H), 8.02 (s, 1H), 7.24 (s, 1H), 7.14 (d, J = 8.0 Hz, 1H), 7.03 (d, J = 8.5 Hz, 2H), 6.88 (d, J = 8.0 Hz, 1H), 6.66 (d, J = 8.5 Hz, 2H), 6.19 (s, 1H), 4.85 (d, J = 3.3 Hz, 1H), 4.32 (br s, 1H), 4.16 (br d, J = 3.8 Hz, 1H), 3.46 (q, J = 6.1 Hz, 2H), 3.03 (br d, J = 6.0 Hz, 1H), 2.99-2.92 (m, 1H), 2.67 (br t, J = 7.5 Hz, 2H), 2.03 (br s, 3H), 1.84-1.69 (m, 9H), 1.63 (br s, 6H), 1.15-0.99 (m, 3H), 0.78-0.69 (m, 3H). |
| 8 | 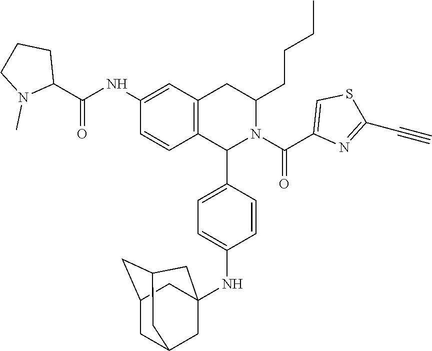 [SMILES: C#Cc1nc(C(=O)N2C(CCCC)Cc3cc(NC(=O)C4CCCN4C)ccc3C2c2ccc(NC34CC5CC(CC(C5)C3)C4)cc2)cs1] | |
| 8a | 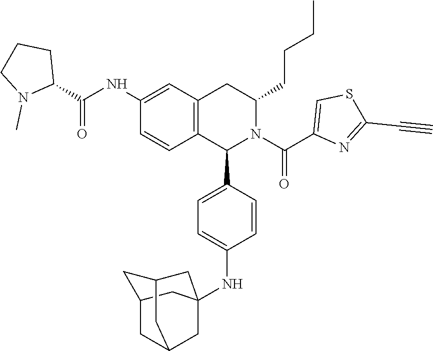 [SMILES: C#Cc1nc(C(=O)N2[C@@H](CCCC)Cc3cc(NC(=O)[C@H]4CCCN4C)ccc3[C@@H]2c2ccc(NC34CC5CC(CC(C5)C3)C4)cc2)cs1] | ¹H NMR (400 MHz, CHLOROFORM-d) δ 7.41-7.35 (m, 2H), 7.31 (t, J = 7.4 Hz, 3H), 7.25-7.20 (m, 1H), 7.01-6.88 (m, 3H), 6.79 (d, J = 8.3 Hz, 1H), 6.66 (d, J = 8.5 Hz, 2H), 6.41 (s, 1H), 4.63 (s, 1H), 3.74 (br d, J = 13.3 Hz, 1H), 3.40 (d, J = 13.8 Hz, 1H), 3.08-2.97 (m, 1H), 2.81- 2.59 (m, 2H), 2.07 (br s, 3H), 1.82 (d, J = 2.5 Hz, 6H), 1.70-1.57 (m, 11H), 1.52 (s, 10H), 1.35-1.29 (m, 2H), 1.16 (qd, J = 7.0, 14.0 Hz, 3H), 0.85-0.80 (m, 4H). |
| 9 | 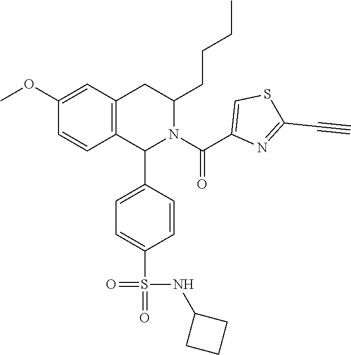 [SMILES: C#Cc1nc(C(=O)N2C(CCCC)Cc3cc(OC)ccc3C2c2ccc(S(=O)(=O)NC3CCC3)cc2)cs1] | |
| 9a | 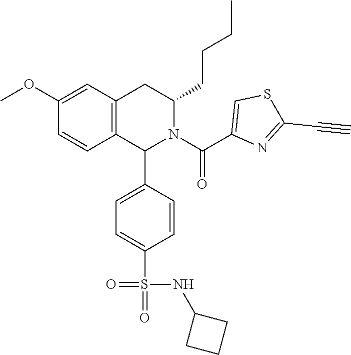 [SMILES: C#Cc1nc(C(=O)N2C(c3ccc(S(=O)(=O)NC4CCC4)cc3)c3ccc(OC)cc3C[C@@H]2CCCC)cs1] | LC-MS (m/z): 564.1 [M + H]⁺; ¹H NMR (400 MHz, DMSO-d₆) δ 8.32 (br s, 1H), 7.86 (br d, J = 7.03 Hz, 1H), 7.70-7.24 (m, 5H), 6.86-6.75 (m, 2H), 6.34 (br s, 1H), 5.10-4.84 (m, 2H), 3.71 (s, 3H), 3.54 (br d, J = 8.78 Hz, 1H), 3.17 (br d, J = 14.81 Hz, 1H), 2.84 (br d, J = 15.81 Hz, 1H), 1.93-1.55 (m, 4H), 1.51-1.37 (m, 2H), 1.31-0.91 (m, 6H), 0.66 (br s, 3H). |
| 10 | 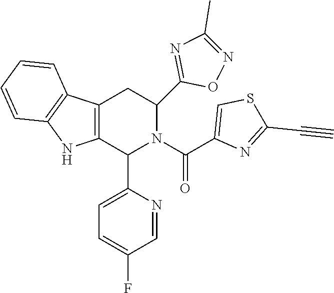 [SMILES: C#Cc1nc(C(=O)N2C(c3nc(C)no3)Cc3c([nH]c4ccccc34)C2c2ccc(F)cn2)cs1] | |
| 10a | 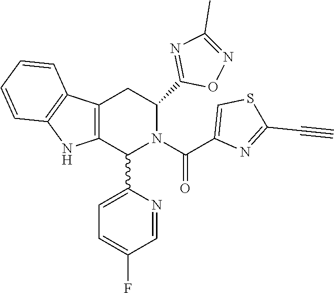 [SMILES: C#Cc1nc(C(=O)N2C(c3ccc(F)cn3)c3[nH]c4ccccc4c3C[C@@H]2c2nc(C)no2)cs1] | LC-MS (m/z): 507.1 [M + Na]⁺; Diastereomer 10aA: ¹H NMR (500 MHz, CHLOROFORM-d) δ 9.31-8.90 (m, 1H), 8.46 (br s, 1H), 8.17-8.06 (m, 1H), 7.64-7.27 (m, 5H), 7.23-7.03 (m, 2H), 6.76 (br s, 1H), 5.17-4.93 (m, 1H), 3.96- 3.69 (m, 2H), 3.66-3.39 (m, 1H), 2.49- 2.42 (m, 1H), 2.28-2.15 (m, 2H) Diastereomer 10aB: ¹H NMR (400 MHz, DMSO-d₆) δ 11.19-10.82 (m, 1H), 8.72- 8.05 (m, 2H), 7.75-7.26 (m, 4H), 7.21- 6.90 (m, 3H), 6.73-6.38 (m, 1H), 5.10- 5.01 (m, 1H), 3.72-3.60 (m, 1H), 3.48- 3.28 (m, 1H), 2.11-1.98 (m, 3H) |
| 11 | 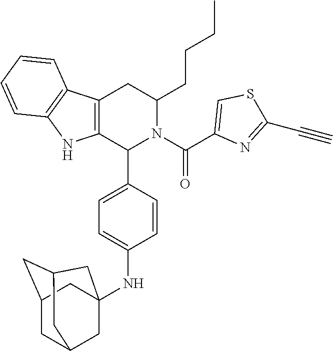 [SMILES: C#Cc1nc(C(=O)N2C(CCCC)Cc3c([nH]c4ccccc34)C2c2ccc(NC34CC5CC(CC(C5)C3)C4)cc2)cs1] | |
| 11a | 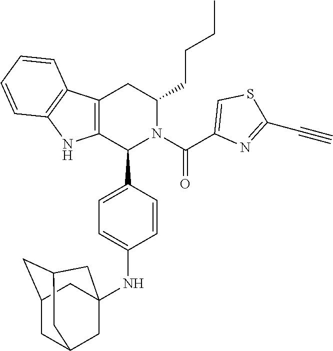 [SMILES: C#Cc1nc(C(=O)N2[C@@H](CCCC)Cc3c([nH]c4ccccc34)[C@@H]2c2ccc(NC34CC5CC(CC(C5)C3)C4)cc2)cs1] | LC-MS (m/z): 589.8 [M + H]⁺; ¹H NMR (400 MHz, DMSO-d₆) δ 10.76 (s, 1H), 8.10 (br d, J = 1.0 Hz, 1H), 7.44 (d, J = 7.8 Hz, 1H), 7.22 (d, J = 7.8 Hz, 1H), 7.08-6.91 (m, 4H), 6.65 (br d, J = 8.5 Hz, 2H), 6.35-6.02 (m, 1H), 5.03 (s, 1H), 4.80 (s, 1H), 3.30 (br s, 1H), 3.11- 2.96 (m, 1H), 2.01 (br s, 3H), 1.80 (br d, J = 1.8 Hz, 6H), 1.60 (br s, 6H), 1.34- 0.87 (m, 5H), 0.72 (br s, 3H). |
| 12 | 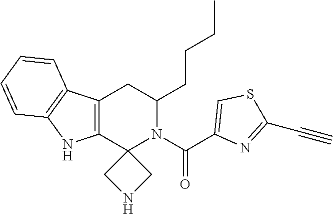 [SMILES: C#Cc1nc(C(=O)N2C(CCCC)Cc3c([nH]c4ccccc34)C23CNC3)cs1] | |
| 12a | 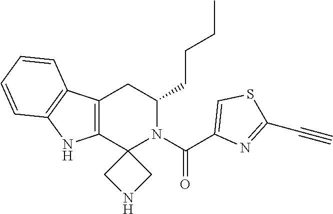 [SMILES: C#Cc1nc(C(=O)N2[C@@H](CCCC)Cc3c([nH]c4ccccc34)C23CNC3)cs1] | ¹H NMR (400 MHz, CHLOROFORM-d) δ 12.15-11.94 (m, 1H), 11.48-11.41 (m, 1H), 8.13-8.08 (m, 1H), 7.97-7.85 (m, 1H), 7.65-7.39 (m, 2H), 7.29 (br d, J = 1.0 Hz, 1H), 7.25 (d, J = 1.0 Hz, 1H), 7.18-7.11 (m, 1H), 5.10-4.74 (m, 5H), 3.58 (s, 1H), 3.37-3.24 (m, 1H), 2.88- 2.78 (m, 1H), 1.52-1.32 (m, 3H), 1.17- 0.98 (m, 4H), 0.79-0.68 (m, 3H). |
| 13 | 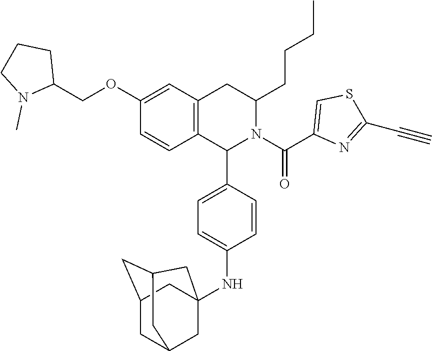 [SMILES: C#Cc1nc(C(=O)N2C(CCCC)Cc3cc(OCC4CCCN4C)ccc3C2c2ccc(NC34CC5CC(CC(C5)C3)C4)cc2)cs1] | |
| 13a | 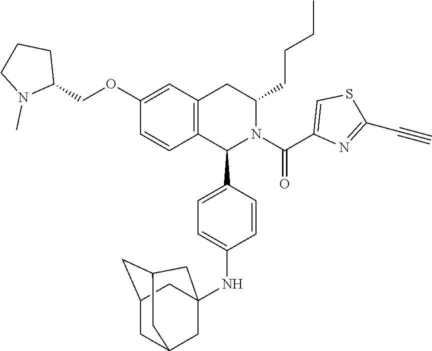 [SMILES: C#Cc1nc(C(=O)N2[C@@H](CCCC)Cc3cc(OC[C@H]4CCCN4C)ccc3[C@@H]2c2ccc(NC34CC5CC(CC(C5)C3)C4)cc2)cs1] | LC-MS (m/z): 663.4 [M + H]⁺; ¹H NMR (400 MHz, DMSO-d₆) δ 10.81 (br s, 2H), 8.34-7.85 (m, 1H), 7.64- 7.16 (m, 5H), 6.95-6.85 (m, 2H), 6.50- 6.38 (m, 1H), 4.99 (br s, 1H), 4.87 (s, 1H), 4.47-4.22 (m, 2H), 3.76 (br s, 1H), 3.60 (br s, 1H), 3.00-2.82 (m, 5H), 2.38- 2.06 (m, 4H), 2.05-1.76 (m, 9H), 1.69- 1.57 (m, 3H), 1.53 (br d, J = 12.0 Hz, 3H), 1.43-0.92 (m, 6H), 0.73 (br s, 3H) |
| 14 | 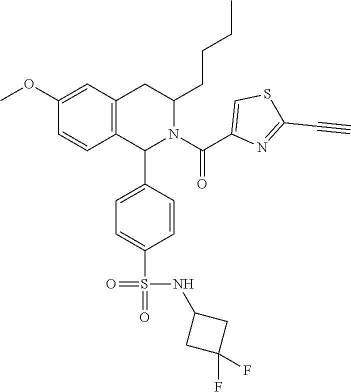 [SMILES: C#Cc1nc(C(=O)N2C(CCCC)Cc3cc(OC)ccc3C2c2ccc(S(=O)(=O)NC3CC(F)(F)C3)cc2)cs1] | |
| 14a | 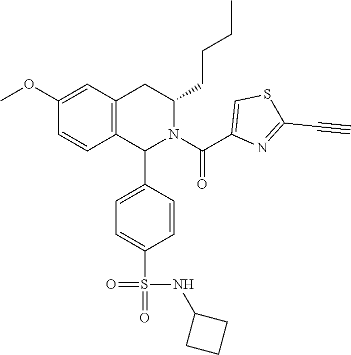 | LC-MS (m/z): 600.1 [M + H]⁺; ¹H NMR (400 MHz, DMSO-d₆) δ 8.32 (s, 1H), 8.12-7.89 (m, 1H), 7.72-7.28 (m, 5H), 6.86-6.76 (m, 2H), 6.71-6.61 (m, 1H), 6.35 (br s, 1H), 5.10-4.88 (m, 2H), 3.71 (s, 3H), 3.53-3.41 (m, 1H), 3.19 (br d, J = 13.88 Hz, 1H), 2.85 (br d, J = 16.01 Hz, 1H), 2.74-2.55 (m, 2H), 2.33 (br s, 2H), 1.61-0.88 (m, 6H), 0.87-0.60 (m, 3H). |
| 15 | 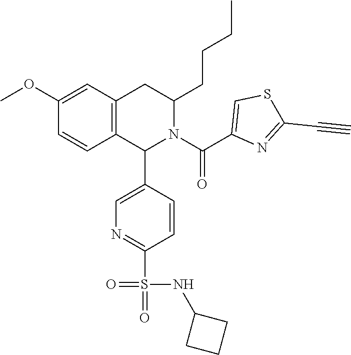 [SMILES: C#Cc1nc(C(=O)N2C(CCCC)Cc3cc(OC)ccc3C2c2ccc(S(=O)(=O)NC3CCC3)nc2)cs1] | |
| 15a | 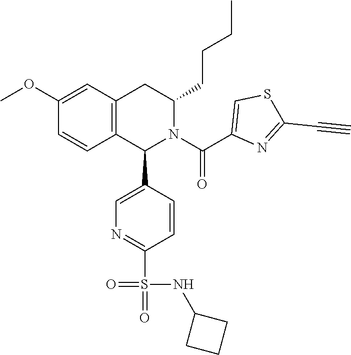 [SMILES: C#Cc1nc(C(=O)N2[C@@H](CCCC)Cc3cc(OC)ccc3[C@@H]2c2ccc(S(=O)(=O)NC3CCC3)nc2)cs1] | LC-MS (m/z): 565.2 [M + H]⁺; ¹H NMR (400 MHz, DMSO-d₆) δ 8.70 (br s, 1H), 8.20 (br s, 1H), 7.93 (br d, J = 7.6 Hz, 1H), 7.84-7.71 (m, 2H), 7.42 (d, J = 8.4 Hz, 1H), 6.88-6.79 (m, 2H), 6.46 (br s, 1H), 4.97 (br s, 1H), 4.87 (s, 1H), 3.75 (s, 4H), 3.22 (br d, J = 16.4 Hz, 1H), 2.91 (br dd, J = 2.0, 16.0 Hz, 1H), 1.92 (br d, J = 6.4 Hz, 2H), 1.86-1.74 (m, 2H), 1.56-1.42 (m, 2H), 1.20-0.92 (m, 6H), 0.80-0.64 (m, 3H). |
| 16 | 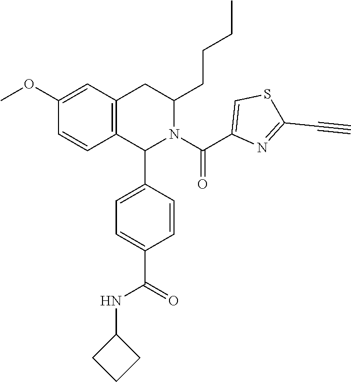 [SMILES: C#Cc1nc(C(=O)N2C(CCCC)Cc3cc(OC)ccc3C2c2ccc(C(=O)NC3CCC3)cc2)cs1] | |
| 16a | 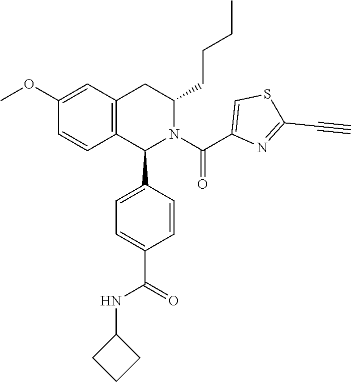 [SMILES: C#Cc1nc(C(=O)N2[C@@H](CCCC)Cc3cc(OC)ccc3[C@@H]2c2ccc(C(=O)NC3CCC3)cc2)cs1] | LC-MS (m/z): 550.2 [M + Na]⁺; ¹H NMR (400 MHz, DMSO-d₆) δ 8.47 (d, J = 7.8 Hz, 1H), 8.30 (s, 1H), 7.81- 7.72 (m, 1H), 7.71-7.57 (m, 2H), 7.54- 7.42 (m, 2H), 7.41-7.09 (m, 1H), 6.80 (br s, 2H), 6.59-6.27 (m, 1H), 5.16- 4.77 (m, 2H), 4.45-4.22 (m, 1H), 3.71 (s, 3H), 3.23-3.11 (m, 1H), 2.94-2.74 (m, 1H), 2.23-2.09 (m, 2H), 2.07-1.94 (m, 2H), 1.70-1.57 (m, 2H), 1.34-0.87 (m, 6H), 0.65 (br s, 2H). |
| 17 | 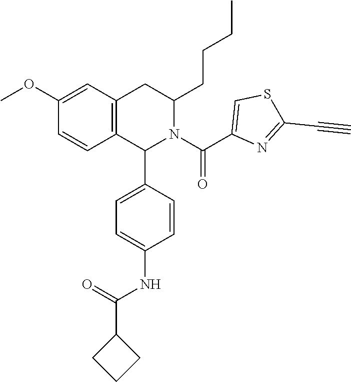 [SMILES: C#Cc1nc(C(=O)N2C(CCCC)Cc3cc(OC)ccc3C2c2ccc(NC(=O)C3CCC3)cc2)cs1] | |
| 17a | 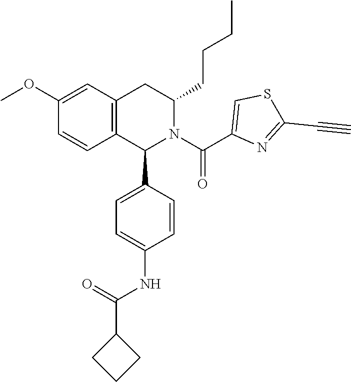 [SMILES: C#Cc1nc(C(=O)N2[C@@H](CCCC)Cc3cc(OC)ccc3[C@@H]2c2ccc(NC(=O)C3CCC3)cc2)cs1] | LC-MS (m/z): 528.2 [M + H]⁺; ¹H NMR (400 MHz, CHLOROFORM-d) δ 7.76-8.03 (m, 1H), 7.50-7.59 (m, 1H), 7.38-7.47 (m, 1H), 7.29-7.36 (m, 1H), 6.87 (br s, 4H), 6.81 (br s, 3H), 6.38- 6.47 (m, 1H), 5.33-5.52 (m, 1H), 4.81- 4.99 (m, 1H), 4.60-4.75 (m, 1H), 4.38- 4.50 (m, 1H), 3.74-3.86 (m, 3H), 3.37- 3.62 (m, 1H), 2.93-3.28 (m, 2H), 2.58- 2.89 (m, 1H), 2.30-2.45 (m, 2H), 2.11- 2.26 (m, 2H), 1.94-2.00 (m, 1H), 1.81- 1.91 (m, 1H), 1.24-1.35 (m, 2H), 0.95- 1.21 (m, 4H), 0.66-0.91 (m, 4H). |
| 18 | 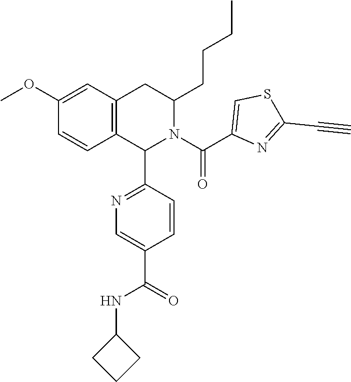 [SMILES: C#Cc1nc(C(=O)N2C(CCCC)Cc3cc(OC)ccc3C2c2ccc(C(=O)NC3CCC3)cn2)cs1] | |
| 18a | 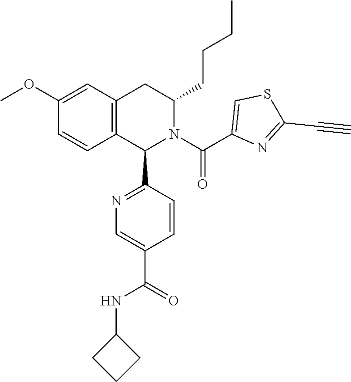 [SMILES: C#Cc1nc(C(=O)N2[C@@H](CCCC)Cc3cc(OC)ccc3[C@@H]2c2ccc(C(=O)NC3CCC3)cn2)cs1] | LC-MS (m/z): 529.1 [M + H]⁺; ¹H NMR (400 MHz, DMSO-d₆) δ 8.93- 8.60 (m, 2H), 8.27 (s, 1H), 8.10 (br d, J = 7.0 Hz, 1H), 7.92 (br d, J = 7.8 Hz, 1H), 7.74-7.64 (m, 1H), 7.54-7.35 (m, 1H), 7.15 (br d, J = 8.0 Hz, 1H), 6.83-6.73 (m, 2H), 6.53 (s, 1H), 6.29 (s, 1H), 5.08- 4.92 (m, 2H), 4.45-4.28 (m, 1H), 3.70 (s, 3H), 3.51 (br dd, J = 3.9, 14.9 Hz, 1H), 3.28-3.19 (m, 1H), 2.83 (br d, J = 14.6 Hz, 1H), 2.19 (br d, J = 7.5 Hz, 2H), 2.06-1.93 (m, 2H), 1.72-1.57 (m, 2H), 1.56-0.91 (m, 6H), 0.69 (br t, J = 6.5 Hz, 3H). |
| 19 | 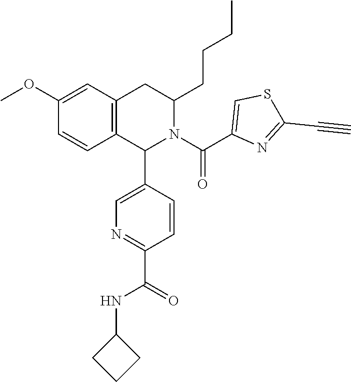 [SMILES: C#Cc1nc(C(=O)N2C(CCCC)Cc3cc(OC)ccc3C2c2ccc(C(=O)NC3CCC3)nc2)cs1] | |
| 19a | 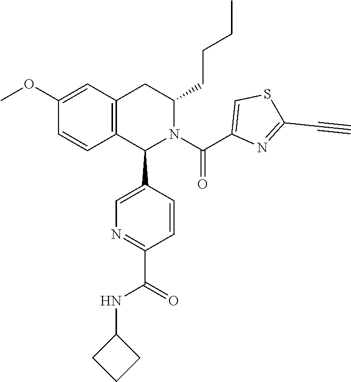 [SMILES: C#Cc1nc(C(=O)N2[C@@H](CCCC)Cc3cc(OC)ccc3[C@@H]2c2ccc(C(=O)NC3CCC3)nc2)cs1] | LC-MS (m/z): 529.3 [M + H]⁺; ¹H NMR (400 MHz, DMSO-d₆) δ 8.66- 8.87 (m, 2H), 8.24-8.44 (m, 1H), 7.74- 7.99 (m, 2H), 7.34-7.55 (m, 1H), 6.72- 6.89 (m, 2H), 6.32-6.46 (m, 1H), 4.86- 5.12 (m, 2H), 4.27-4.49 (m, 1H), 3.66- 3.78 (m, 3H), 3.14-3.30 (m, 1H), 2.80- 2.96 (m, 1H), 2.02-2.23 (m, 4H), 1.49- 1.71 (m, 2H), 0.64-1.27 (m, 10H). |
| 20 | 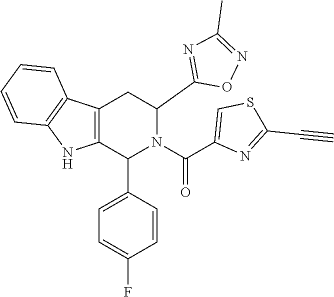 [SMILES: C#Cc1nc(C(=O)N2C(c3nc(C)no3)Cc3c([nH]c4ccccc34)C2c2ccc(F)cc2)cs1] | |
| 20a | 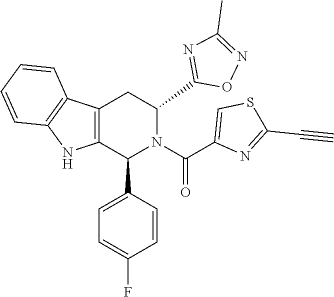 [SMILES: C#Cc1nc(C(=O)N2[C@@H](c3ccc(F)cc3)c3[nH]c4ccccc4c3C[C@@H]2c2nc(C)no2)cs1] | LC-MS (m/z): 506.1 [M + Na]⁺; ¹H NMR (400 MHz, DMSO-d₆) δ 10.82 (br s, 1H), 8.22 (s, 1H), 7.60-7.50 (m, 3H), 7.26 (d, J = 8.0 Hz, 1H), 7.19-6.97 (m, 4H), 6.82-6.61 (m, 1H), 6.17-5.47 (m, 1H), 4.90 (s, 1H), 3.72-3.62 (m, 1H), 3.61-3.49 (m, 1H), 2.24 (s, 3H). |
| 21 | 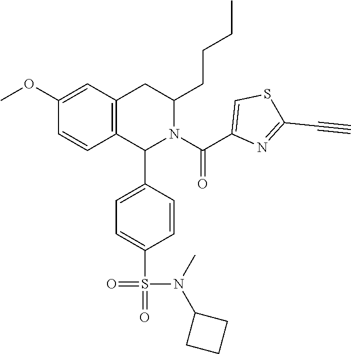 [SMILES: C#Cc1nc(C(=O)N2C(CCCC)Cc3cc(OC)ccc3C2c2ccc(S(=O)(=O)N(C)C3CCC3)cc2)cs1] | |
| 21a | 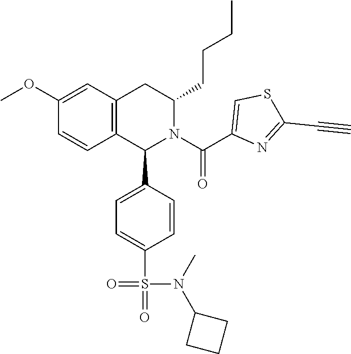 [SMILES: C#Cc1nc(C(=O)N2[C@@H](CCCC)Cc3cc(OC)ccc3[C@@H]2c2ccc(S(=O)(=O)N(C)C3CCC3)cc2)cs1] | LC-MS (m/z): 578.2 [M + H]⁺; ¹H NMR (400 MHz, DMSO-d₆) δ 8.33 (br s, 1 H), 7.93-7.22 (m, 6H), 6.82 (s, 2H), 6.62-6.34 (m, 1H), 5.11-4.87 (m, 2H), 3.93-3.80 (m, 1H), 3.72 (s, 3H), 3.18-3.08 (m, 1H), 2.85 (br d, J = 14.8 Hz, 1H), 2.56 (br s, 3H), 1.97 (br d, J = 8.3 Hz, 2H), 1.83 (br s, 2H), 1.55-1.46 (m, 2H), 1.28-0.95 (m, 6H), 0.85-0.61 (m, 3H). |
| 22 | 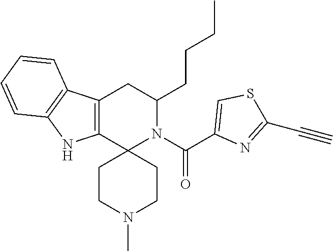 [SMILES: C#Cc1nc(C(=O)N2C(CCCC)Cc3c([nH]c4ccccc34)C23CCN(C)CC3)cs1] | |
| 22a | 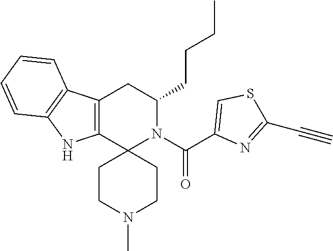 [SMILES: C#Cc1nc(C(=O)N2[C@@H](CCCC)Cc3c([nH]c4ccccc34)C23CCN(C)CC3)cs1] | LC-MS (m/z): 447.1 [M + H]⁺; ¹H NMR (400 MHz, CHLOROFORM-d) δ 11.07 (br s, 1H), 8.79 (s, 1H), 7.78 (s, 1H), 7.48 (t, J = 7.5 Hz, 2H), 7.19 (t, J = 7.6 Hz, 1H), 7.13-7.07 (m, 1H), 4.70- 4.53 (m, 1H), 3.54 (s, 1H), 3.50 (br s, 1H), 3.34-3.15 (m, 4H), 3.02 (br s, 1H), 2.81 (br d, J = 15.1 Hz, 3H), 2.74 (s, 3H), 2.21 (br s, 1H), 1.62-1.38 (m, 2H), 1.13- 1.00 (m, 4H), 0.73 (br t, J = 6.8 Hz, 3H). |
| 23 | 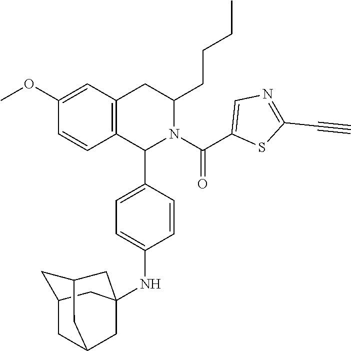 [SMILES: C#Cc1ncc(C(=O)N2C(CCCC)Cc3cc(OC)ccc3C2c2ccc(NC34CC5CC(CC(C5)C3)C4)cc2)s1] | |
| 23a | 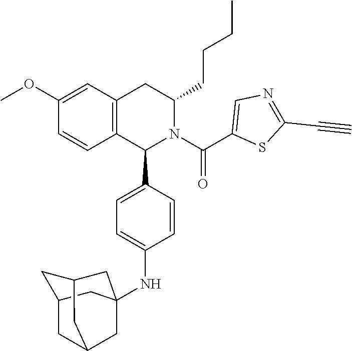 [SMILES: C#Cc1ncc(C(=O)N2[C@@H](CCCC)Cc3cc(OC)ccc3[C@@H]2c2ccc(NC34CC5CC(CC(C5)C3)C4)cc2)s1] | ¹H NMR (400 MHz, CHLOROFORM-d) δ = 8.16-7.84 (m, 1H), 7.53-7.28 (m, 3H), 7.23-6.95 (m, 2H), 6.86-6.44 (m, 3H), 5.55-4.84 (m, 1H), 3.80 (s, 3H), 3.60-3.44 (m, 1H), 3.21-2.95 (m, 1H), 2.92-2.62 (m, 1H), 1.96-1.66 (m, 10H), 1.58-1.39 (m, 6H), 1.38-0.93 (m, 7H), 0.92-0.60 (m, 3H). |
| 24 | 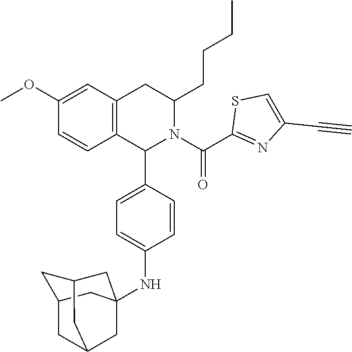 [SMILES: C#Cc1csc(C(=O)N2C(CCCC)Cc3cc(OC)ccc3C2c2ccc(NC34CC5CC(CC(C5)C3)C4)cc2)n1] | |
| 24a | 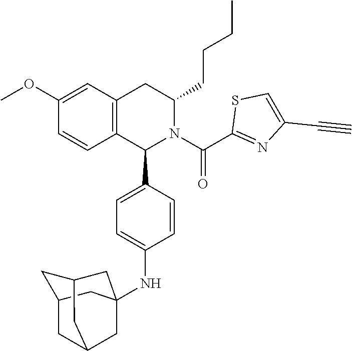 [SMILES: C#Cc1csc(C(=O)N2[C@@H](CCCC)Cc3cc(OC)ccc3[C@@H]2c2ccc(NC34CC5CC(CC(C5)C3)C4)cc2)n1] | LC-MS (m/z): 580.4 [M + H]⁺; ¹H NMR (400 MHz, CHLOROFORM-d) δ 10.75-11.11 (m, 2H), 7.66-7.75 (m, 1H), 7.28-7.46 (m, 4H), 7.08-7.21 (m, 1H), 6.65-6.85 (m, 2H), 6.43-6.55 (m, 1H), 5.83-5.95 (m, 1H), 4.80-4.90 (m, 1H), 3.72-3.90 (m, 3H), 2.94-3.21 (m, 2H), 2.71-2.88 (m, 1H), 1.70-1.89 (m, 9H), 1.13-1.49 (m, 12 H), 0.70-0.91 (m, 3H). |
| 25 | 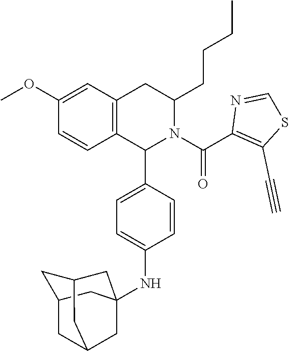 [SMILES: C#Cc1scnc1C(=O)N1C(CCCC)Cc2cc(OC)ccc2C1c1ccc(NC23CC4CC(CC(C4)C2)C3)cc1] | |
| 25a | 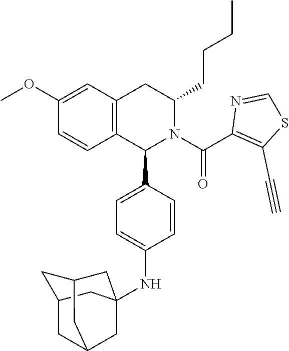 [SMILES: C#Cc1scnc1C(=O)N1[C@@H](CCCC)Cc2cc(OC)ccc2[C@@H]1c1ccc(NC23CC4CC(CC(C4)C2)C3)cc1] | |
| 26 | 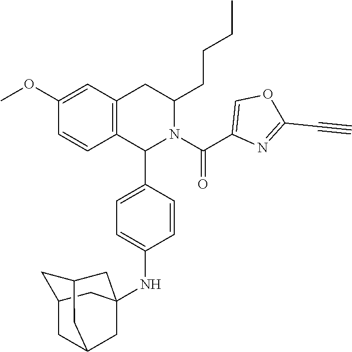 [SMILES: C#Cc1nc(C(=O)N2C(CCCC)Cc3cc(OC)ccc3C2c2ccc(NC34CC5CC(CC(C5)C3)C4)cc2)co1] | |
| 26a | 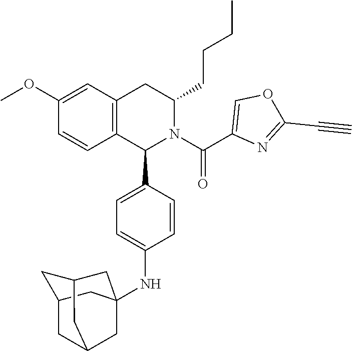 [SMILES: C#Cc1nc(C(=O)N2[C@@H](CCCC)Cc3cc(OC)ccc3[C@@H]2c2ccc(NC34CC5CC(CC(C5)C3)C4)cc2)co1] | LC-MS (m/z): 564.4 [M + H]⁺; ¹H NMR (400 MHz, DMSO-d₆) δ 10.58- 11.01 (m, 2H), 8.31-8.79 (m, 1H), 7.12- 7.58 (m, 5H), 6.61-6.95 (m, 2H), 6.22- 6.44 (m, 1H), 4.61-5.39 (m, 2H), 3.64- 3.85 (m, 3H), 2.77-3.13 (m, 2H), 1.99- 2.22 (m, 3H), 1.39-1.92 (m, 13H), 0.97-1.30 (m, 6H), 0.57-0.85 (m, 3H). |
| 27 | 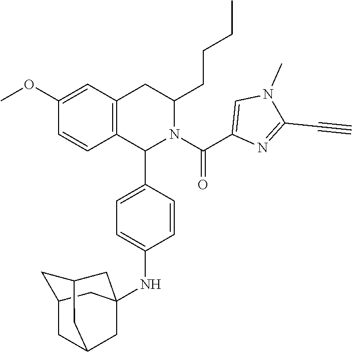 [SMILES: C#Cc1nc(C(=O)N2C(CCCC)Cc3cc(OC)ccc3C2c2ccc(NC34CC5CC(CC(C5)C3)C4)cc2)cn1C] | |
| 27a | 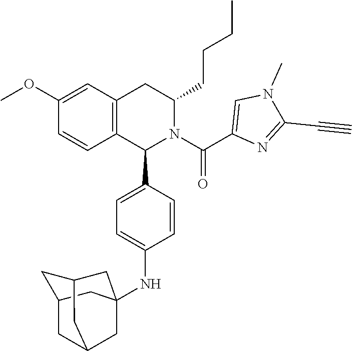 [SMILES: C#Cc1nc(C(=O)N2[C@@H](CCCC)Cc3cc(OC)ccc3[C@@H]2c2ccc(NC34CC5CC(CC(C5)C3)C4)cc2)cn1C] | LC-MS (m/z): 577.3 [M + H]⁺; ¹H NMR (400 MHz, DMSO-d₆) δ 10.94 (br s, 2H), 7.69 (s, 1H), 7.40 (br d, J = 8.4 Hz, 3H), 7.30 (br d, J = 8.0 Hz, 2H), 6.84- 6.75 (m, 2H), 6.32-5.16 (m, 1H), 4.56 (br s, 1H), 3.79-3.72 (m, 3H), 3.72- 3.65 (m, 3H), 3.05 (br d, J = 11.2 Hz, 1H), 2.85 (br d, J = 15.6 Hz, 1H), 2.10 (br s, 3H), 1.85 (br s, 6H), 1.66-1.49 (m, 6H), 1.44-0.99 (m, 6H), 0.78-0.66 (m, 3H). |
| 28 | 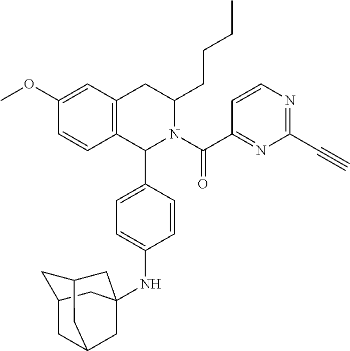 [SMILES: C#Cc1nccc(C(=O)N2C(CCCC)Cc3cc(OC)ccc3C2c2ccc(NC34CC5CC(CC(C5)C3)C4)cc2)n1] | |
| 28a | 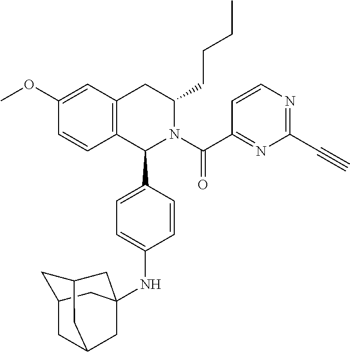 [SMILES: C#Cc1nccc(C(=O)N2[C@@H](CCCC)Cc3cc(OC)ccc3[C@@H]2c2ccc(NC34CC5CC(CC(C5)C3)C4)cc2)n1] | LC-MS (m/z): 597.3 [M + Na]⁺; ¹H NMR (400 MHz, CHLOROFORM-d) δ 11.04-10.87 (m, 2H), 8.88 (d, J = 5.0 Hz, 1H), 8.22 (br d, J = 4.5 Hz, 1H), 7.58 (d, J = 5.0 Hz, 1H), 7.50-7.28 (m, 4H), 7.11-7.00 (m, 1H), 6.94 (d, J = 5.0 Hz, 1H), 6.79 (dd, J = 2.4, 8.3 Hz, 1H), 6.68- 6.61 (m, 1H), 6.52-6.44 (m, 1H), 4.97 (br d, J = 9.3 Hz, 1H), 3.82-3.74 (m, 3H), 3.26-3.18 (m, 1H), 3.13-3.00 (m, 1H), 2.94-2.58 (m, 1H), 1.90-1.66 (m, 15H), 1.48-1.37 (m, 7H), 0.87-0.85 (m, 1H), 0.64 (br t, J = 6.4 Hz, 2H). |
| 29 | 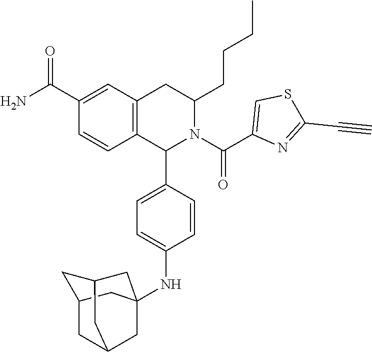 [SMILES: C#Cc1nc(C(=O)N2C(CCCC)Cc3cc(C(N)=O)ccc3C2c2ccc(NC34CC5CC(CC(C5)C3)C4)cc2)cs1] | |
| 29a | 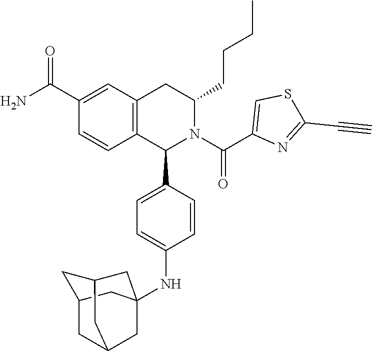 [SMILES: C#Cc1nc(C(=O)N2[C@@H](CCCC)Cc3cc(C(N)=O)ccc3[C@@H]2c2ccc(NC34CC5CC(CC(C5)C3)C4)cc2)cs1] | LC-MS (m/z): 593.2 [M + H]⁺; ¹H NMR (400 MHz, DMSO-d₆) δ 11.18- 10.57 (m, 1H), 8.40-8.29 (m, 1H), 8.02- 7.85 (m, 1H), 7.83-7.05 (m, 8H), 6.69- 6.37 (m, 1H), 5.06 (br s, 2H), 3.26 (br d, J = 8.8 Hz, 1H), 3.02-2.84 (m, 1H), 2.08 (br s, 3H), 1.80 (br s, 6H), 1.67- 1.42 (m, 6H), 1.27-0.55 (m, 9H). |
| 30 | 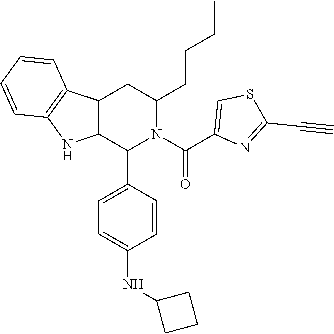 [SMILES: C#Cc1nc(C(=O)N2C(CCCC)CC3c4ccccc4NC3C2c2ccc(NC3CCC3)cc2)cs1] | |
| 30a | 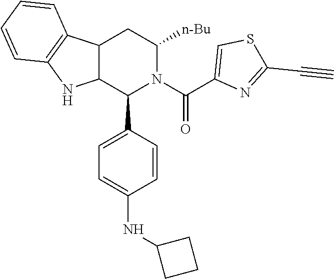 | |

### Compositions

The present disclosure also provides a composition (e.g., a pharmaceutical composition) comprising a compound provided herein (e.g., a compound of any one of Formulas I, I-a, IA, IB, IC, ID, IE, IF, IG, IH, IJ, IK, IL, IM, IN, IP, IQ, IR, II, IIA, IIB, IIC, IID, IIE, IIF, IIG, IIH, IIJ, IIK, IIL, IIM, IIN, or IIP), or a salt, ester, tautomer, prodrug, zwitterionic form, or stereoisomer thereof. In some embodiments, a provided composition comprises a compound provided herein, or a pharmaceutically acceptable salt thereof. For example, the present disclosure provides a pharmaceutical composition comprising a compound provided herein (e.g., a compound of any one of Formulas I, I-a, IA, IB, IC, ID, IE, IF, IG, IH, IJ, IK, IL, IM, IN, IP, IQ, IR, II, IIA, IIB, IIC, IID, IIE, IIF, IIG, IIH, IIJ, IIK, IIL, IIM, IIN, or ITP), or a salt, ester, tautomer, prodrug, zwitterionic form, or stereoisomer thereof, together with a pharmaceutically acceptable carrier. In some embodiments, a provided pharmaceutical composition comprises a compound provided herein or a pharmaceutically acceptable salt thereof, together with a pharmaceutically acceptable carrier.

In some embodiments, the pharmaceutical composition is formulated for oral administration. In some embodiments, the oral pharmaceutical formulation is selected from a tablet and a capsule.

In some embodiments, the pharmaceutical composition is formulated for parenteral administration. In some embodiments, the pharmaceutical composition is formulated for intravenous administration. In some embodiments, the pharmaceutical composition is formulated for subcutaneous administration.

While it may be possible for certain compounds provided herein (e.g., a compound of any one of Formulas I, I-a, IA, IB, IC, ID, IE, IF, IG, IH, IJ, IK, IL, IM, IN, IP, IQ, IR, II, IIA, IIB, IIC, IID, IIE, IIF, IIG, IIH, IIJ, IIK, IIL, IIM, IIN, or IIP), or a salt, ester, tautomer, prodrug, zwitterionic form, or stereoisomer thereof, to be administered as the raw chemical, compounds may additionally or alternatively be provided in a pharmaceutical formulation. Accordingly, provided herein are pharmaceutical formulations which comprise one or more compounds disclosed herein (e.g., a compound of any one of Formulas I, I-a, IA, IB, IC, ID, IE, IF, IG, IH, IJ, IK, IL, IM, IN, IP, IQ, IR, II, IIA, IIB, IIC, IID, IIE, IIF, IIG, IIH, IIJ, IIK, IIL, IIM, IIN, or IIP), or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration selected. Any of the well-known techniques, carriers, and excipients may be used as suitable and as understood in the art. The pharmaceutical compositions disclosed herein may be manufactured in any suitable manner known, e.g., by means of conventional mixing, dissolving, granulating, dragee-making, levigating, emulsifying, encapsulating, entrapping or compression processes.

A pharmaceutical formulation provided herein can be suitable for oral, parenteral (including subcutaneous, intradermal, intramuscular, intravenous, intraarticular, and intramedullary), intraperitoneal, transmucosal, transdermal, rectal, and topical (including dermal, buccal, sublingual, and intraocular) administration. The most suitable route may depend on, for example, the condition and disorder of the subject to which the pharmaceutical formulation will be administered. A pharmaceutical formulation can be provided in a unit dosage form. A pharmaceutical formulation can be prepared by any suitable method. A method of preparing a pharmaceutical formulation may comprise bringing a compound provided herein (e.g., a compound of any one of Formulas I, I-a, IA, IB, IC, ID, IE, IF, IG, IH, IJ, IK, IL, IM, IN, IP, IQ, IR, II, IIA, IIB, IIC, IID, IIE, IIF, IIG, IIH, IIJ, IIK, IIL, IIM, IIN, or IIP), or a pharmaceutically acceptable salt, ester, amide, prodrug or solvate thereof (“active ingredient”) in contact with one or more pharmaceutically acceptable carriers (e.g., accessory ingredients). In general, the formulations are prepared by uniformly and intimately bringing into association the active ingredient with liquid carriers or finely divided solid carriers or both and then, if necessary, shaping the product into the desired formulation.

Pharmaceutical formulations of compounds provided herein (e.g., compounds of any one of Formulas I, I-a, IA, IB, IC, ID, IE, IF, IG, IH, IJ, IK, IL, IM, IN, IP, IQ, IR, II, IIA, IIB, IIC, IID, IIE, IIF, IIG, IIH, IIJ, IIK, IIL, IIM, IIN, or IIP in any available form (e.g., salt, ester, tautomer, prodrug, zwitterionic form, stereoisomer etc.)) may be provided as discrete units. For example, a formulation suitable for oral administration may be provided as capsules, cachets, and/or tablets containing a predetermined amount of the compound in any suitable form (e.g., the active ingredient); as a solution or suspension in a solvent (e.g., aqueous or non-aqueous solvent); as an emulsion (e.g., an oil-in-water liquid emulsion or water-in-oil liquid emulsion); or as a powder or granules. The active ingredient may additionally or alternatively be provided as a bolus, electuary, or paste.

Pharmaceutical preparations suitable for oral administration include tablets, push-fit capsules made of gelatin, as well as soft, sealed capsules made of gelatin and a plasticizer, such as glycerol or sorbitol. Tablets may be made by, for example, compression or molding, optionally with one or more accessory ingredients, such as one or more pharmaceutically acceptable excipients. Compressed tablets may be prepared by, for example, compressing in a suitable machine the active ingredient in a free-flowing form such as a powder or granules, optionally mixed with binders, inert diluents, or lubricating, surface active or dispersing agents. Molded tablets may be made by, for example, molding in a suitable machine a mixture of the powdered compound moistened with an inert liquid diluent. The tablets may optionally be coated or scored and may be formulated to provide slow or controlled release of the active ingredient therein. All formulations for oral administration should be in dosages suitable for such administration. The push-fit capsules can contain the active ingredients in admixture with, for example, one or more fillers such as lactose, one or more binders such as one or more starches, and/or one or more lubricants such as talc or magnesium stearate and, optionally, one or more stabilizers. In soft capsules, the active compounds may be dissolved or suspended in suitable liquids, such as fatty oils, liquid paraffin, or liquid polyethylene glycols. Stabilizers and other elements may also be added. Dragee cores are provided with suitable coatings. For this purpose, concentrated sugar solutions may be used, which may optionally contain a gum, gelling agent, polymer, solvent, or combination thereof. Dyestuffs or pigments may be added to the tablets or dragee coatings for identification or to characterize different combinations of active compound doses.

A pharmaceutical composition comprising a compound provided herein (e.g., a compound of any one of Formulas I, I-a, IA, IB, IC, ID, IE, IF, IG, IH, IJ, IK, IL, IM, IN, IP, IQ, IR, II, IIA, IIB, IIC, IID, IIE, IIF, IIG, IIH, IIJ, IIK, IIL, IIM, IIN, or IIP), or a form thereof (e.g., salt, ester, tautomer, prodrug, zwitterionic form, stereoisomer, etc.), may be formulated for parenteral administration by injection, e.g., by bolus injection or continuous infusion. Formulations for injection may be presented in unit dosage form, e.g., in ampoules, vials, or in multi-dose containers, with an added preservative. The compositions may take such forms as suspensions, solutions, or emulsions in oily or aqueous vehicles, and may contain formulating agents such as suspending, stabilizing, and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, prior (e.g., immediately prior) to use. Extemporaneous injection solutions and suspensions may be prepared from sterile powders, granules and tablets of the kind previously described.

A pharmaceutical composition comprising a compound provided herein (e.g., a compound of any one of Formulas I, I-a, IA, IB, IC, ID, IE, IF, IG, IH, IJ, IK, IL, IM, IN, IP, IQ, IR, II, IIA, IIB, IIC, IID, IIE, IIF, IIG, IIH, IIJ, IIK, IIL, IIM, IIN, or IIP), or a form thereof (e.g., salt, ester, tautomer, prodrug, zwitterionic form, stereoisomer etc.), may be formulated as a solution for injection, which solution may be an aqueous or non-aqueous (oily) sterile solution and may comprise one or more antioxidants, thickening agents, suspending agents, buffers, solutes, and/or bacteriostats. The addition of one or more such additives may render the formulation isotonic with the blood of the intended recipient (e.g., subject or patient). Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described elsewhere herein, the compounds provided herein (e.g., compounds of any one of Formulas I, I-a, IA, IB, IC, ID, IE, IF, IG, IH, IJ, IK, IL, IM, IN, IP, IQ, IR, II, IIA, IIB, IIC, IID, IIE, IIF, IIG, IIH, IIJ, IIK, IIL, IIM, IIN, or IIP in any suitable form (e.g., salt, ester, tautomer, prodrug, zwitterionic form, stereoisomer, etc.)), may also be formulated as a depot preparation. Such long-acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical composition comprising a compound provided herein (e.g., a compound of any one of Formulas I, I-a, IA, IB, IC, ID, IE, IF, IG, IH, IJ, IK, IL, IM, IN, IP, IQ, IR, II, IIA, IIB, IIC, IID, IIE, IIF, IIG, IIH, IIJ, IIK, IIL, IIM, IIN, or IIP) or a form thereof (e.g., salt, ester, tautomer, prodrug, zwitterionic form, stereoisomer, etc.) that is suitable for buccal or sublingual administration may take the form of tablets, lozenges, pastilles, or gels. Such compositions may comprise the active ingredient in a flavored basis such as sucrose and acacia or tragacanth. A pharmaceutical composition comprising a compound provided herein or a form thereof (e.g., salt, ester, tautomer, prodrug, zwitterionic form, stereoisomer, etc.) that is suitable for rectal administration may be formulated as a suppository or retention enema and may comprise a medium such as, for example, cocoa butter, polyethylene glycol, or other glycerides.

Certain compounds provided herein (e.g., a compound of any one of Formulas I, I-a, IA, IB, IC, ID, IE, IF, IG, IH, IJ, IK, IL, IM, IN, IP, IQ, IR, II, IIA, IIB, IIC, IID, IIE, IIF, IIG, IIH, IIJ, IIK, IIL, IIM, IIN, or IIP) or a form thereof (e.g., salt, ester, tautomer, prodrug, zwitterionic form, stereoisomer, etc.) may be formulated for non-systemic administration, such as topical administration. This includes the application of a compound disclosed herein, or a form thereof, externally to the epidermis or the buccal cavity and the instillation of such a compound, or a form thereof, into the ear, eye and nose, such that the compound, or a form thereof, does not significantly enter the blood stream. In contrast, systemic administration refers to oral, intravenous, intraperitoneal, and intramuscular administration.

Formulations suitable for topical administration include liquid or semi-liquid preparations suitable for penetration through the skin to the site of inflammation such as gels, liniments, lotions, creams, ointments, or pastes, and drops suitable for administration to the eye, ear, or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

For administration by inhalation, compounds (e.g., compounds of any one of Formulas I, I-a, IA, IB, IC, ID, IE, IF, IG, IH, IJ, IK, IL, IM, IN, IP, IQ, IR, II, IIA, IIB, IIC, IID, IIE, IIF, IIG, IIH, IIJ, IIK, IIL, IIM. IIN, or IIP) or forms thereof (e.g., salt, ester, tautomer, prodrug, zwitterionic form, stereoisomer, etc.) may be conveniently delivered from an insufflator, nebulizer pressurized packs, or other convenient means of delivering an aerosol spray. Pressurized packs may comprise a suitable propellant such as dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount. Alternatively, for administration by inhalation or insufflation, the compounds provided herein may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Exemplary unit dosage formulations are those containing an effective dose, as described herein, or an appropriate fraction thereof, of the active ingredient (e.g., a compound provided herein (e.g., a compound of any one of Formulas I, I-a, IA, IB, IC, ID, IE, IF, IG, IH, IJ, IK, IL, IM, IN, IP, IQ, IR, II, IIA, IIB, IIC, IID, IIE, IIF, IIG, IIH, IIJ, IIK, IIL, IIM, IIN, or IIP), or a salt, ester, tautomer, prodrug, zwitterionic form, or stereoisomer thereof).

It should be understood that in addition to the ingredients particularly described elsewhere herein, the formulations described herein may include other useful agents having regard to the type of formulation in question, for example those suitable for oral administration may include flavoring agents.

Compounds (e.g., compounds of any one of Formulas I, I-a, IA, IB, IC, ID, IE, IF, IG, IH, IJ, IK, IL, IM, IN, IP, IQ, IR, II, IIA, IIB, IIC, IID, IIE, IIF, IIG, IIH, IIJ, IIK, IIL, IIM, IIN, or IIP) or forms thereof (e.g., salt, ester, tautomer, prodrug, zwitterionic form, stereoisomer, etc.) may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

### Methods

The present disclosure also provides a method of modulating (e.g., inhibiting) GPX4 comprising contacting GPX4, or a cell comprising GPX4, with a compound provided herein (e.g., a compound of any one of Formulas I, I-a, IA, IB, IC, ID, IE, IF, IG, IH, IJ, IK, IL, IM, IN, IP, IQ, IR, II, IIA, IIB, IIC, IID, IIE, IIF, IIG, IIH, IIJ, IIK, IIL, IIM, IIN, or IIP) or a form thereof (e.g., salt, ester, tautomer, prodrug, zwitterionic form, stereoisomer, mixture of stereoisomers, isotopically enriched analogs, etc.). The present disclosure also provides a method of inducing ferroptosis comprising contacting a cell with a compound provided herein (e.g., a compound of any one of Formulas I, I-a, IA, IB, IC, ID, IE, IF, IG, IH, IJ, IK, IL, IM, IN, IP, IQ, IR, II, IIA, IIB, IIC, IID, IIE, IIF, IIG, IIH, IIJ, IIK, IIL, IIM, IIN, or IIP) or a form thereof (e.g., salt, ester, tautomer, prodrug, zwitterionic form, stereoisomer, mixture of stereoisomers, isotopically enriched analogs, etc.). In some embodiments, the methods comprise contacting a cell with the compound or form thereof, or a composition comprising the same. In some embodiments, the cell is located within a subject, such as a human subject. In some embodiments, the subject has a disease, disorder, or condition, such as a cancer (e.g., as described herein). In some embodiments, the cell is a cancer cell.

In some embodiments, a compound provided herein (e.g., compounds of any one of Formulas I, I-a, IA, IB, IC, ID, IE, IF, IG, IH, IJ, IK, IL, IM, IN, IP, IQ, IR, II, IIA, IIB, IIC, IID, IIE, IIF, IIG, IIH, IIJ, IIK, IIL, IIM, IIN, or IIP) is used in a method of inhibiting GPX4 in a cell, comprising contacting a cell with an effective amount of a compound or composition described herein to inhibit GPX4 in the cell. In certain embodiments, the cell is a cancer cell. In certain embodiments, the method comprises administering an effective amount of a compound or composition described herein to a patient in need thereof.

In some embodiments, a compound provided herein (e.g., compounds of any one of Formulas I, I-a, IA, JB, IC, ID, IE, IF, IG, IH, IJ, IK, IL, IM, IN, IP, IQ, IR, II, IIA, IIB, IIC, IID, IIE, IIF, IIG, IIH, IIJ, IIK, IIL, IIM, IIN, or IIP) is used in a method of inducing ferroptosis in a cell comprising contacting the cell with an effective amount of a compound or composition provided herein. In certain embodiments, the method comprises administering an effective amount of a compound or composition described herein to a patient in need thereof.

The present disclosure also provides a method of treating a disease, disorder, or condition, such as a cancer (e.g., as described herein) in a subject in need thereof using a compound provided herein (e.g., a compound of any one of Formulas I, I-a, IA, IB, IC, ID, IE, IF, IG, IH, IJ, IK, IL, IM, IN, IP, IQ, IR, IL, IIA, IIB, IIC, IID, IIE, IIF, IIG, IIH, IIJ, IIK, IIL, IIM, IIN, or IIP) or a form thereof (e.g., salt, ester, tautomer, prodrug, zwitterionic form, stereoisomer, mixture of stereoisomers, isotopically enriched analogs, etc.). For example, the present disclosure provides a method comprising providing (e.g., administering) to a subject (e.g., patient) in need thereof an effective amount of a compound provided herein (e.g., a compound of any one of Formulas I, I-a, IA, IB, IC, ID, IE, IF, IG, IH, IJ, 1K, IL, IM, IN, IP, IQ, IR, II, IIA, IIB, IIC, IID, IIE, IIF, IG, IIH, IIJ, IIK, IIL, IIM, IIN, or IIP) or a form thereof (e.g., salt, ester, tautomer, prodrug, zwitterionic form, stereoisomer, mixture of stereoisomers, isotopically enriched analogs, etc.). The present disclosure also provides methods of treating a disease, disorder, or condition in a subject in need thereof using a pharmaceutical composition comprising a compound provided herein (e.g., a compound of any one of Formulas I, I-a, IA, IB, IC, ID, IE, IF, IG, IH, IJ, IK, IL, IM, IN, IP, IQ, IR, II, IIA, IIB, IIC, IID, IIE, IIF, IIG, IIH, IIJ, IIK, IIL, IIM, IIN, or IIP) or a form thereof (e.g., salt, ester, tautomer, prodrug, zwitterionic form, stereoisomer, mixture of stereoisomers, isotopically enriched analogs, etc.). For example, the present disclosure provides a method comprising providing (e.g., administering) to a subject (e.g., patient) in need thereof a pharmaceutical composition comprising an effective amount of a compound provided herein (e.g., a compound of any one of Formulas I, I-a, IA, IB, IC, ID, IE, IF, IG, IH, IJ, IK, IL, IM, IN, IP, IQ, IR, II, IIA, IIB, IIC, IID, IIE, IIF, IIG, IIH, IIJ, IIK, IIL, IIM, IIN, or IIP) or a form thereof (e.g., salt, ester, tautomer, prodrug, zwitterionic form, stereoisomer, mixture of stereoisomers, isotopically enriched analogs, etc.). In some embodiments, the subject is known to have (e.g., has previously been diagnosed with) a disease, disorder, or condition such as a cancer.

The present disclosure also provides a compound as provided herein (e.g., a compound of any one of Formulas I, I-a, IA, IB, IC, ID, IE, IF, IG, IH, IJ, IK, IL, IM, IN, IP, IQ, IR, II, IIA, IIB, IIC, IID, IIE, IIF, IIG, IIH, IIJ, IIK, IIL, IIM, IIN, or IIP) or a form thereof (e.g., salt, ester, tautomer, prodrug, zwitterionic form, stereoisomer, mixture of stereoisomers, isotopically enriched analogs, etc.), or a pharmaceutical composition comprising any of the foregoing compounds and a pharmaceutically acceptable excipient, for use as a medicament, such as a medicament for the treatment of a disease, disorder, or condition (e.g., a cancer). The present disclosure also provides a compound as provided herein (e.g., a compound of any one of Formulas I, I-a, IA, IB, IC, ID, IE, IF, IG, IH, IJ, IK, IL, IM, IN, IP, IQ, IR, II, IIA, IIB, IIC, IID, IIE, IIF, IIG, IIH, IIJ, IIK, IIL, IIM, IIN, or IIP) or a form thereof (e.g., salt, ester, tautomer, prodrug, zwitterionic form, stereoisomer, mixture of stereoisomers, isotopically enriched analogs, etc.), or a pharmaceutical composition comprising any of the foregoing compounds and a pharmaceutically acceptable excipient, for use in the manufacture of a medicament for the treatment of a disease, disorder, or condition (e.g., a cancer) in a subject in need thereof.

The present disclosure also provides the use of a compound provided herein (e.g., a compound of any one of Formulas I, I-a, IA, IB, IC, ID, IE, IF, IG, IH, IJ, IK, IL, IM, IN, IP, IQ, IR, II, IIA, IIB, IIC, IID, IIE, IIF, IIG, IIH, IIJ, IIK, IIL, IIM, IIN, or IIP) or a form thereof (e.g., salt, ester, tautomer, prodrug, zwitterionic form, stereoisomer, mixture of stereoisomers, isotopically enriched analogs, etc.), or a pharmaceutical composition comprising any of the foregoing compounds and a pharmaceutically acceptable excipient, for the treatment of a disease, disorder, or condition (e.g., a cancer, as described herein) in a subject in need thereof.

The present disclosure also provides the use of a compound provided herein (e.g., a compound of any one of Formulas I, I-a, IA, IB, IC, ID, IE, IF, IG, IH, IJ, IK, IL, IM, IN, IP, IQ, IR, II, IIA, IIB, IIC, IID, IIE, IIF, IIG, IIH, IIJ, IIK, IIL, IIM, IIN, or IIP) or a form thereof (e.g., salt, ester, tautomer, prodrug, zwitterionic form, stereoisomer, mixture of stereoisomers, isotopically enriched analogs, etc.), or a pharmaceutical composition comprising any of the foregoing compounds and a pharmaceutically acceptable excipient, in the manufacture of a medicament for treating a disease, disorder, or condition (e.g., a cancer, as described herein) in a subject in need thereof.

In some embodiments, a compound provided herein (e.g., a compound of any one of Formulas I, I-a, IA, IB, IC, ID, IE, IF, IG, IH, IJ, IK, IL, IM, IN, IP, IQ, IR, II, IIA, IIB, IIC, IID, IIE, IIF, IIG, IIH, IIJ, IIK, IIL, IIM, IIN, or IIP) or a form thereof (e.g., salt, ester, tautomer, prodrug, zwitterionic form, stereoisomer, mixture of stereoisomers, isotopically enriched analogs, etc.) is used in a method of treating a disease, disorder, or condition such as a cancer. In some embodiments, the method comprises administering to a subject in need thereof a therapeutically effective amount any of the compounds described herein, or a composition comprising the same (e.g., as described herein).

The present disclosure also provides a method comprising administering a therapeutically effective amount of a compound provided herein (e.g., a compound of any one of Formulas I, I-a, IA, IIB, IC, ID, IE, IF, IG, IH, IJ, IK, IL, IM, IN, IP, IQ, IR, II, IIA, IIB, IIC, IID, IIE, IIF, IIG, IIH, IIJ, IIK, IIL, IIM, IIN, or IIP) or a form thereof (e.g., salt, ester, tautomer, prodrug, zwitterionic form, stereoisomer, mixture of stereoisomers, isotopically enriched analogs, etc.) to a subject (e.g., patient) (e.g., subject in need thereof), thereby ameliorating, reducing, eliminating, ceasing, delaying the progression of, or improving one or more symptoms of the subject, such as one or more symptoms of a disease, disorder, or condition (e.g., a cancer).

In some embodiments, administering a therapeutically effective amount of a compound provided herein (e.g., a compound of any one of Formulas I, I-a, IA, IB, IC, ID, IE, IF, IG, IH, IJ, IK, IL, IM, IN, IP, IQ, IR, II, IIA, IIB, IIC, IID, IIE, IIF, IG, IIH, IIJ, IIK, IIL, IIM, IIN, or IIP) or a form thereof (e.g., salt, ester, tautomer, prodrug, zwitterionic form, stereoisomer, mixture of stereoisomers, isotopically enriched analogs, etc.) slows or prevents growth of a tumor. In some embodiments, administering a therapeutically effective amount of a compound provided herein (e.g., a compound of any one of Formulas I, I-a, IA, IB, IC, ID, IE, IF, IG, IH, IJ, IK, IL, IM, IN, IP, IQ, IR, II, IIA, IIB, IIC, IID, IIE, IIF, IIG, IIH, IIJ, IIK, IIL, IIM, IIN, or IIP) or a form thereof (e.g., salt, ester, tautomer, prodrug, zwitterionic form, stereoisomer, mixture of stereoisomers, isotopically enriched analogs, etc.) results in shrinkage of a tumor (e.g., tumor regression). In some embodiments, administering a therapeutically effective amount of a compound provided herein (e.g., a compound of any one of Formulas I, I-a, IA, IB, IC, ID, IE, IF, IG, IH, IJ, IK, IL, IM, IN, IP, IQ, IR, II, IIA, IIB, IIC, IID, IIE, IIF, IIG, IIH, IIJ, IIK, IIL, IIM, IIN, or IIP) or a form thereof (e.g., salt, ester, tautomer, prodrug, zwitterionic form, stereoisomer, mixture of stereoisomers, isotopically enriched analogs, etc.) results in at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, or 99% regression of a tumor, such as for a period of one or more weeks (e.g., at least about 1, 2, 3, 4, 5, 6, 7, 8, 9, 10, 11, 12, or more weeks), a period of one or more months (e.g., at least about 1, 2, 3, 4, 5, 6, 7, 8, 9, 10, 11, 12, or more months), or a period of one or more years (e.g., at least about 1, 2, 3, or more years). In some embodiments, administering a therapeutically effective amount of a compound provided herein (e.g., a compound of any one of Formulas I, I-a, IA, IB, IC, ID, IE, IF, IG, IH, IJ, IK, IL, IM, IN, IP, IQ, IR, II, IIA, IIB, IIC, IID, IIE, IIF, IIG, IIH, IIJ, IIK, IIL, IIM, IIN, or IIP) or a form thereof (e.g., salt, ester, tautomer, prodrug, zwitterionic form, stereoisomer, mixture of stereoisomers, isotopically enriched analogs, etc.) stabilizes a tumor. In some embodiments, administering a therapeutically effective amount of a compound provided herein (e.g., a compound of any one of Formulas I, I-a, IA, IB, IC, ID, IE, IF, IG, IH, IJ, IK, IL, IM, IN, IP, IQ, IR, II, IIA, IIB, IIC, IID, IIE, IIF, IIG, IIH, IIJ, IIK, IIL, IIM, IIN, or IIP) or a form thereof (e.g., salt, ester, tautomer, prodrug, zwitterionic form, stereoisomer, mixture of stereoisomers, isotopically enriched analogs, etc.) stabilizes a tumor for a period of one or more weeks (e.g., at least about 1, 2, 3, 4, 5, 6, 7, 8, 9, 10, 11, 12, or more weeks), a period of one or more months (e.g., at least about 1, 2, 3, 4, 5, 6, 7, 8, 9, 10, 11, 12, or more months), or a period of one or more years (e.g., at least about 1, 2, 3, or more years).

In certain embodiments, a compound described herein (e.g., compounds of any one of Formulas I, I-a, IA, IB, IC, ID, IE, IF, IG, IH, IJ, IK, IL, IM, IN, IP, IQ, IR, II, IIA, IIB, IIC, IID, IIE, IIF, IIG, IIH, IIJ, IIK, IIL, IIM, IIN, or IIP) or a form thereof (e.g., salt, ester, tautomer, prodrug, zwitterionic form, stereoisomer, mixture of stereoisomers, isotopically enriched analogs, etc.) is used in a method of treating cancer in a subject in need thereof, comprising administering to a subject having cancer a therapeutically effective amount of a ferroptosis inducing compound disclosed herein. Various cancers for treatment with the compounds and forms thereof provided herein include, but are not limited to, adrenocortical cancer, anal cancer, biliary cancer, bladder cancer, bone cancer, gliomas, astrocytoma, neuroblastoma, breast cancer, cervical cancer, colon cancer, endometrial cancer, esophageal cancer, head and neck cancer, intestinal cancer, liver cancer, lung cancer, oral cancer, ovarian cancer, pancreatic cancer, renal cancer, prostate cancer, salivary gland cancer, skin cancer, stomach cancer, testicular cancer, throat cancer, thyroid cancer, uterine cancer, vaginal cancer, sarcoma, and soft tissue carcinomas. In certain embodiments, the compound is used to treat pancreatic cancer.

In certain embodiments, the cancer is renal cell carcinoma (RCC), pancreatic cancer, lung cancer, breast cancer, or prostate cancer. In certain embodiments, provided is a method for treating renal cell carcinoma (RCC) in a patient in need thereof, comprising administering an effective amount of a compound or composition provided herein. In certain embodiments, provided is a method for treating pancreatic cancer in a patient in need thereof, comprising administering an effective amount of a compound or composition provided herein. In certain embodiments, provided is a method for treating lung cancer in a patient in need thereof, comprising administering an effective amount of a compound or composition provided herein. In certain embodiments, provided is a method for treating breast cancer in a patient in need thereof, comprising administering an effective amount of a compound or composition provided herein. In certain embodiments, provided is a method for treating prostate cancer in a patient in need thereof, comprising administering an effective amount of a compound or composition provided herein.

In certain embodiments, provided is a method for treating a malignant solid tumor in a patient in need thereof, comprising administering an effective amount of a compound or composition provided herein to the patient. In certain embodiments, the malignant solid tumor is a carcinoma. In certain embodiments, the malignant solid tumor is a lymphoma. In certain embodiments, the malignant solid tumor is a sarcoma.

In certain embodiments, the cancer is selected from, among others, adrenocortical cancer, anal cancer, biliary cancer, bladder cancer, bone cancer (e.g., osteosarcoma), brain cancer (e.g., gliomas, astrocytoma, neuroblastoma, etc.), breast cancer, cervical cancer, colon cancer, endometrial cancer, esophageal cancer, head and neck cancer, hematologic cancer (e.g., leukemia and lymphoma), intestinal cancer (small intestine), liver cancer, lung cancer (e.g., bronchial cancer, small cell lung cancer, non-small cell lung cancer, etc.), oral cancer, ovarian cancer, pancreatic cancer, renal cancer, prostate cancer, salivary gland cancer, skin cancer (e.g., basal cell carcinoma, melanoma), stomach cancer, testicular cancer, throat cancer, thyroid cancer, uterine cancer, vaginal cancer, sarcoma, and soft tissue carcinomas. In certain embodiments, the cancer is renal cell carcinoma (RCC). In certain embodiments, the cancer is pancreatic cancer. In certain embodiments, the cancer is lung cancer. In certain embodiments, the cancer is breast cancer. In certain embodiments, the cancer is prostate cancer.

In certain embodiments, the cancer is pancreatic cancer. In certain embodiments, the pancreatic cancer for treatment with the compounds is pancreatic adenocarcinoma or metastatic pancreatic cancer. In certain embodiments, the cancer is stage I, stage II, stage III, or stage IV pancreatic adenocarcinoma.

In certain embodiments, the cancer is lung cancer. In certain embodiments, the lung cancer for treatment with the compounds is small cell lung cancer or non-small cell lung cancer. In certain embodiments, the non-small cell lung cancer is an adenocarcinoma, squamous cell carcinoma, or large cell carcinoma. In certain embodiments, the lung cancer is metastatic lung cancer.

In certain embodiments, the cancer is a hematologic cancer. In certain embodiments, the hematologic cancer is selected from acute lymphoblastic leukemia (ALL), acute myeloid leukemia (AML), lymphoma (e.g., Hodgkin's lymphoma, Non-Hodgkin's lymphoma, Burkitt's lymphoma), chronic lymphocytic leukemia (CLL), chronic myelogenous leukemia (CML), Hairy Cell chronic myelogenous leukemia (CML), and multiple myeloma.

In certain embodiments, the cancer is a leukemia selected from acute lymphoblastic leukemia (ALL), acute myeloid leukemia (AML), chronic lymphocytic leukemia (CLL), chronic myelogenous leukemia (CML), Hairy Cell chronic myelogenous leukemia (CML), and multiple myeloma.

In certain embodiments, the cancer is a lymphoma selected from Hodgkin's lymphoma, Non-Hodgkin's lymphoma, and Burkitt's lymphoma.

In certain embodiments, the cancer is a cancer characterized by mesenchymal features or mesenchymal phenotype. In some cancers, gain of mesenchymal features is associated with migratory (e.g., intravasation) and invasiveness of cancers. Mesenchymal features can include, among others, enhanced migratory capacity, invasiveness, elevated resistance to apoptosis, and increased production of extracellular matrix (ECM) components. In addition to these physiological characteristics, the mesenchymal features can include expression of certain biomarkers, including among others, E-cadherin, N-cadherin, integrins, FSP-1, α-SMA, vimentin, β-catenin, collagen I, collagen II, collagen III, collagen IV, fibronectin, laminin 5, SNAIL-1, SNAIL-2, Twist-1, Twist-2, and Lef-1. In certain embodiments, the cancer selected for treatment with the compounds herein include, among others, breast cancer, lung cancer, head and neck cancer, prostate cancer, and colon cancer. In certain embodiments, the mesenchymal features can be inherent to the cancer type or induced by or selected for by treatment of cancers with chemotherapy and/or radiation therapy.

In certain embodiments, the cancer is identified as having or determined to have an activating or oncogenic RAS activity. In certain embodiments, the RAS is K-RAS, H-RAS or N-RAS. In certain embodiments, the activating or oncogenic RAS is an activating or oncogenic RAS mutation.

In certain embodiments, the cancer is determined to have or identified as having an activating or oncogenic RAS activity. In certain embodiments, the activating or oncogenic RAS activity is an activating or oncogenic RAS mutations. In certain embodiments, the activating or oncogenic RAS activity is an activating or activating K-RAS activity, particularly an activating or oncogenic K-RAS mutation. In certain embodiments, the activating or oncogenic RAS activity is an activating or activating N-RAS activity, particularly an activating or oncogenic N-RAS mutation. In certain embodiments, the activating or oncogenic RAS activity is an activating or activating H-RAS activity, particularly an activating or oncogenic H-RAS mutation.

In certain embodiments, the compounds and forms thereof provided herein can be used to treat a cancer that is refractory to one or more other chemotherapeutic agents, particularly cytotoxic chemotherapeutic agents; or treat a cancer resistant to radiation treatment. In certain embodiments, the compounds are used to treat cancers that have developed tolerance to chemotherapeutic agents activating other cell death pathways, such as apoptosis, mitotic catastrophe, necrosis, senescence and/or autophagy.

In certain embodiments, the cancer is identified as being refractory or resistant to chemotherapy. In certain embodiments, the cancer is refractory or resistant to one or more of alkylating agents, anti-cancer antibiotic agents, antimetabolic agents (e.g., folate antagonists, purine analogs, pyrimidine analogs, etc.), topoisomerase inhibiting agents, anti-microtubule agents (e.g., taxanes, vinca alkaloids), hormonal agents (e.g., aromatase inhibitors), plant-derived agents and their synthetic derivatives, anti-angiogenic agents, differentiation inducing agents, cell growth arrest inducing agents, apoptosis inducing agents, cytotoxic agents, agents affecting cell bioenergetics i.e., affecting cellular ATP levels and molecules/activities regulating these levels, biologic agents, e.g., monoclonal antibodies, kinase inhibitors and inhibitors of growth factors and their receptors.

In certain embodiments, the cancer is a cancer identified as being refractory or resistant to one or more of afatinib, afuresertib, alectinib, alisertib, alvocidib, amsacrine, amonafide, anuvatinib, axitinib, azacitidine, azathioprine, bafetinib, barasertib, bendamustine, bleomycin, bosutinib, bortezomib, busulfan, cabozantinib, camptothecin, canertinib, capecitabine, cabazitaxel, carboplatin, carmustine, cenisertib, ceritinib, chlorambucil, cisplatin, cladribine, clofarabine, crenolanib, crizotinib, cyclophosphamide, cytarabine, dabrafenib, dacarbazine, dacomitinib, dactinomycin, danusertib, dasatinib, daunorubicin, decitabine, dinaciclib, docetaxel, dovitinib, doxorubicin, epirubicin, epitinib, eribulin mesylate, errlotinib, etirinotecan, etoposide, everolimus, exemestane, floxuridine, fludarabine, fluorouracil, gefitinib, gemcitabine, hydroxyurea, ibrutinib, icotinib, idarubicin, ifosfamide, imatinib, imetelstat, ipatasertib, irinotecan, ixabepilone, lapatinib, lenalidomide, lestaurtinib, lomustine, lucitanib, masitinib, mechlorethamine, melphalan, mercaptopurine, methotrexate, midostaurin, mitomycin, mitoxantrone, mubritinib, nelarabine, neratinib, nilotinib, nintedanib, omacetaxine mepesuccinate, orantinib, oxaliplatin, paclitaxel, palbociclib, palifosfamide tris, pazopanib, pelitinib, pemetrexed, pentostatin, plicamycin, ponatinib, poziotinib, pralatrexate, procarbazine, quizartinib, raltitrexed, regorafenib, ruxolitinib, seliciclib, sorafenib, streptozocin, sulfatinib, sunitinib, tamoxifen, tandutinib, temozolomide, temsirolimus, teniposide, theliatinib, thioguanine, thiotepa, topotecan, uramustine, valrubicin, vandetanib, vemurafenib (Zelborae), vincristine, vinblastine, vinorelbine, and vindesine.

In certain embodiments, the cancer is identified as being refractory or resistant to one or more chemotherapeutics agents selected from cyclophosphamide, chlorambucil, melphalan, mechlorethamine, ifosfamide, busulfan, lomustine, streptozocin, temozolomide, dacarbazine, cisplatin, carboplatin, oxaliplatin, procarbazine, uramustine, methotrexate, pemetrexed, fludarabine, cytarabine, fluorouracil, floxuridine, gemcitabine, capecitabine, vinblastine, vincristine, vinorelbine, etoposide, paclitaxel, docetaxel, doxorubicin, daunorubicin, epirubicin, idarubicin, mitoxantrone, bleomycin, mitomycin, hydroxyurea, topotecan, irinotecan, amsacrine, teniposide, and erlotinib.

In certain embodiments, the cancer is a cancer resistant to ionizing radiation therapy. The radioresistance of the cancer can be inherent or as a result of radiation therapy. In certain embodiments, the cancers for treatment with the compounds is, among others, a radioresistant adrenocortical cancer, anal cancer, biliary cancer, bladder cancer, bone cancer (e.g., osteosarcoma), brain cancer (e.g., gliomas, astrocytoma, neuroblastoma, etc.), breast cancer, cervical cancer, colon cancer, endometrial cancer, esophageal cancer, head and neck cancer, hematologic cancer (e.g., leukemia and lymphoma), intestinal cancer (small intestine), liver cancer, lung cancer (e.g., bronchial cancer, small cell lung cancer, non-small cell lung cancer, etc.), oral cancer, ovarian cancer, pancreatic cancer, renal cancer, prostate cancer, salivary gland cancer, skin cancer (e.g., basal cell carcinoma, melanoma), stomach cancer, testicular cancer, throat cancer, thyroid cancer, uterine cancer, or vaginal cancer. In certain embodiments, the cancer is pancreatic cancer, breast cancer, glioblastoma, advanced non-small-cell lung cancer, bladder cancer, sarcoma, or soft tissue carcinoma.

### Combination Treatments

The compounds provided herein (e.g., compounds of any one of Formulas I, I-a, IA, IB, IC, ID, IE, IF, IG, IH, IJ, IK, IL, IM, IN, IP, IQ, IR, II, IIA, IIB, IIC, IID, IIE, IIF, IIG, IIH, IIJ, IIK, IIL, IIM, IIN, or IIP) and forms thereof (e.g., salt, ester, tautomer, prodrug, zwitterionic form, stereoisomer, mixture of stereoisomers, isotopically enriched analogs, etc.), or compositions (e.g., pharmaceutical compositions) comprising the same, can be administered in various modes (e.g., orally, topically, or by injection). The amount of active ingredient (e.g., a compound provided herein in any suitable form thereof) administered to a subject (e.g., patient) will be the responsibility of an attendant medical provider. The specific dose level for a given subject (e.g., patient) will depend on a variety of factors including, for example, the activity of the active ingredient administered; the physical attributes of the subject (e.g., age, weight, height, body mass index, general health, co-morbidities, sex, etc.); other characteristics of the subject (e.g., diet, level of exercise, national origin, ethnicity, etc.); time of administration; route of administration; rate of excretion; drug combination; the disease, disorder, or condition being treated; and the severity of the disease, disorder, or condition being treated.

In some embodiments, a compound provided herein (e.g., a compound of any one of Formulas I, I-a, IA, IB, IC, ID, IE, IF, IG, IH, IJ, IK, IL, IM, IN, IP, IQ, IR, II, IIA, IIB, IIC, IID, IIE, IIF, IIG, IIH, IIJ, IIK, IIL, IIM, IIN, or IIP) or a form thereof (e.g., salt, ester, tautomer, prodrug, zwitterionic form, stereoisomer, mixture of stereoisomers, isotopically enriched analogs, etc.) is administered in combination with an additional agent, such as an additional therapeutic agent. For example, if a subject experiences a side effect such as hypertension upon receiving a compound provided herein, or a form thereof, it may be appropriate to administer an additional agent that is effective in managing the side effect, such as an anti-hypertensive agent. In another example, the therapeutic effectiveness of a compound provided herein (e.g., a compound of any one of Formulas I, I-a, IA, IB, IC, ID, IE, IF, IG, IH, IJ, IK, IL, IM, IN, IP, IQ, IR, IL, IIA, IIB, IIC, IID, IIE, IIF, IIG, IIH, IIJ, IIK, IIL, IIM, IIN, or IIP), or a form thereof, may be enhanced by administration of an adjuvant, which adjuvant may itself have only minimal therapeutic benefit, but in combination with another therapeutic agent may provide an enhanced overall therapeutic benefit to a subject. In a further example, the therapeutic benefit of a compound provided herein (e.g., a compound of any one of Formulas I, I-a, IA, IB, IC, ID, IE, IF, IG, IH, IJ, IK, IL, IM, IN, IP, IQ, IR, IL, IIA, IIB, IIC, IID, IE, IIF, IIG, IIH, IIJ, IIK, ILL, IIM, IIN, or IIP), or a form thereof, may be enhanced by administration of the compound, or a form thereof, and an additional agent (which may comprise an additional therapeutic regimen) that also provides a therapeutic benefit. For example, a compound provided herein (e.g., a compound of any one of Formulas I, I-a, IA, IB, IC, ID, IE, IF, IG, IH, IJ, IK, IL, IM, IN, IP, IQ, IR, II, IIA, IIB, IIC, IID, IIE, IIF, IIG, IIH, IIJ, IIK, IIL, IIM, IIN, or IIP), or a form thereof, may be administered in combination with an additional agent that may be effective in the treatment of a disease, disorder, or condition such as a cancer. Generally, the combination of a compound provided herein (e.g., a compound of any one of Formulas I, I-a, IA, IB, IC, ID, IE, IF, IG, IH, IJ, IK, IL, IM, IN, IP, IQ, IR, II, IIA, IIB, IIC, IID, IIE, IIF, IIG, IIH, IIJ, IIK, IIL, IIM, IIN, or IIP), or a form thereof, and one or more additional agents (e.g., therapeutic agents) may enhance the overall benefit experienced by the subject upon either component individually. In some embodiments, the effect may be additive. In some embodiments, the effect may be synergistic.

In certain embodiments, the compounds described herein (e.g., compounds of any one of Formulas I, I-a, IA, IB, IC, ID, IE, IF, IG, IH, IJ, IK, IL, IM, IN, IP, IQ, IR, II, IIA, IIB, IIC, IID, IIE, IIF, IIG, IIH, IIJ, IIK, IIL, IIM, IIN, or IIP) are used in combination with one or more of other (e.g., second therapeutic agent) therapeutic treatments for cancer. In certain embodiments, the compounds can be used as monotherapy, or as further provided below, in a combination therapy with one or more therapeutic treatments, particularly in combination with one or more chemotherapeutic agents. In certain embodiments, the compounds are used in combination with a second therapeutic agent, where the compounds are used at levels that sensitizes the cancer or cancer cell to the second therapeutic agent, for example at levels of the compound that do not cause significant cell death. In certain embodiments, the compounds can be used in combination with radiation therapy, either to sensitize the cells to radiation therapy or as an adjunct to radiation therapy (e.g., at doses sufficient to activate cell death pathway).

In certain embodiments, a subject with cancer is treated with a combination of a compound described herein (e.g., a compound of any one of Formulas I, I-a, IA, IB, IC, ID, IE, IF, IG, IH, IJ, IK, IL, IM, IN, IP, IQ, IR, II, IIA, IIB, IIC, IID, IIE, IIF, IIG, IIH, IIJ, IIK, IIL, IIM, IIN, or IIP) and radiation therapy. In certain embodiments, the method comprises administering to a subject with cancer a therapeutically effective amount of a compound of the disclosure, and adjunctively treating the subject with an effective amount of radiation therapy. In certain embodiments, the compound is administered to the subject in need thereof prior to, concurrently with, or subsequent to the treatment with radiation.

In certain embodiments, the method comprises administering an effective amount of a compound described herein to a subject with cancer to sensitize the cancer to radiation treatment, and administering a therapeutically effective amount of radiation therapy to treat the cancer. In certain embodiments, an effective amount of X-ray and gamma ray is administered to the subject. In certain embodiments, an effective amount of particle radiation is administered to the subject, where the particle radiation is selected from electron beam, proton beam, and neutron beam radiation. In certain embodiments, the radiation therapy is fractionated.

In certain embodiments, a subject with cancer is administered a therapeutically effective amount of a compound described herein, or a first pharmaceutical composition thereof, and adjunctively administered a therapeutically effective amount of a second chemotherapeutic agent, or a second pharmaceutical composition thereof.

In certain embodiments, the second chemotherapeutic agent is selected from an platinating agent, alkylating agent, anti-cancer antibiotic agent, antimetabolic agent (e.g., folate antagonists, purine analogs, pyrimidine analogs, etc.), topoisomerase I inhibiting agent, topoisomerase II inhibiting agent antimicrotubule agent (e.g., taxanes, vinca alkaloids), hormonal agent (e.g., aromatase inhibitors), plant-derived agent and synthetic derivatives thereof, anti-angiogenic agent, differentiation inducing agent, cell growth arrest inducing agent, apoptosis inducing agent, cytotoxic agent, agent affecting cell bioenergetics, i.e., affecting cellular ATP levels and molecules/activitics regulating these levels, anti-cancer biologic agent (e.g., monoclonal antibodies), kinase inhibitors and inhibitors of growth factors and their receptors.

In certain embodiments, the second chemotherapeutic agent is an angiogenesis inhibitor, such as but not limited to, an inhibitor of soluble VEGFR-1, NRP-1, angiopoietin 2, TSP-1, TSP-2, angiostatin and related molecules, endostatin, vasostatin, calreticulin, platelet factor-4, TIMP, CDAI, Meth-1, Meth-2, IFN-α, IFN-β, IFN-γ, CXCL10, IL-4, IL-12, IL-18, prothrombin (kringle domain-2), antithrombin III fragment, prolactin, VEGI, SPARC, osteopontin, maspin, canstatin (a fragment of COL4A2), or proliferin-related protein. In certain embodiments, the angiogenesis inhibitor is bevacizumab (Avastin), itraconazole, carboxyamidotriazole, TNP-470 (an analog of fumagillin), CM101, IFN-α, IL-12, platelet factor-4, suramin, SU5416, thrombospondin, a VEGFR antagonist, an angiostatic steroid plus heparin, cartilage-derived angiogenesis inhibitory factor (CDAI), a matrix metalloproteinase inhibitor, angiostatin, endostatin, 2-methoxyestradiol, tecogalan, tetrathiomolybdate, thalidomide, thrombospondin, prolactin, a αVβ3 inhibitor, linomide, ramucirumab, tasquinimod, ranibizumab, sorafenib (Nexavar), sunitinib (Sutent), pazopanib (Votrient), or everolimus (Afinitor).

In certain embodiments, the second chemotherapeutic agent is a cyclin-dependent kinase (CDK) inhibitor (e.g., a CDK4/CDK6 inhibitor). Examples include, but are not limited to, palbociclib (Ibrance), Ribociclib (optionally further in combination with letrozole), abemaciclib (LY2835219; Verzenio), P1446A-05, and Trilaciclib (G1T28).

In certain embodiments, the second chemotherapeutic agent is a Bruton's tyrosine kinase (BTK) inhibitor, such as but not limited to, Ibrutinib (PCI-32765), acalabrutinib, ONO-4059 (GS-4059), spebrutinib (AVL-292, CC-292), BGB-3111, and HM71224.

In certain embodiments, the second chemotherapeutic agent is a BRAF inhibitor. Examples include, but are not limited to, BAY43-9006 (Sorafenib, Nexavar), PLX-4032 (Vemurafenib), GDC-0879, PLX-4720, dabrafenib and LGX818.

In certain embodiments, the second chemotherapeutic agent is a EGFR inhibitor. Examples include, but are not limited to, gefitinib, erlotinib, afatinib, brigatinib, icotinib, cetuximab, osimertinib, panitumumab, brigatinib, lapatinib, cimaVax-EGF, and veristrat.

In certain embodiments, the second chemotherapeutic agent is a human epidermal growth factor receptor 2 (HER2) inhibitor. Examples include, but are not limited to, trastuzumab, pertuzumab (optionally further in combination with trastuzumab), margetuximab, and NeuVax.

In certain embodiments, disclosed herein is a method of increasing a subject's responsiveness to an immunotherapeutic or immunogenic chemotherapeutic agent, the method comprising administering to the subject in need thereof an effective amount of a compound described herein and an effective amount of an immunotherapeutic agent and/or an immunogenic chemotherapeutic agent. In certain embodiments, the method further includes administering to the subject a lipoxygenase inhibitor. In certain embodiments, the subject has a tumor whose cellular microenvironment is stromal cell rich. In certain embodiments, the administration of compound described herein results in killing one or more stromal cells in the tumor cells' microenvironment. In certain embodiments, the administration of an effective amount of an immunotherapeutic agent and/or an immunogenic chemotherapeutic agent results in killing one or more tumor cells. Also provided herein is a combination comprising a compound described herein and an immunotherapeutic agent, lipoxygenase inhibitor, or immunogenic chemotherapeutic agent. In certain embodiments, the immunotherapeutic agent is selected from a CTLA4, PDL1 or PD1 inhibitor. In certain embodiments, the immunotherapeutic agent can be selected from CTLA4 inhibitor such as ipilimumab, a PD1 inhibitor such as pembrolizumab or nivolumab or a PDL1 inhibitor such as atezolizumab or durvalumab. In certain embodiments, the immunotherapeutic agent is pembrolizumab. In other embodiments, the immunogenic chemotherapeutic agent is a compound selected from anthracycline, doxorubicin, cyclophosphamide, paclitaxel, docetaxel, cisplatin, oxaliplatin or carboplatin. In certain embodiments, provided herein is a combination comprising a compound described herein and a lipoxygenase inhibitor. In certain embodiments, the lipoxygenase inhibitor is selected from PD147176 and/or ML351. In certain embodiments, the lipoxygenase inhibitor may be a 15-lipoxygenase inhibitor (see, e.g., Sadeghian et al., Expert Opinion on Therapeutic Patents, 2015, 26:1, 65-88).

In certain embodiments, the second chemotherapeutic agent is selected from an alkylating agent, including, but not limiting to, adozelesin, altretamine, bendamustine, bizelesin, busulfan, carboplatin, carboquone, carmofur, carmustine, chlorambucil, cisplatin, cyclophosphamide, dacarbazine, estramustine, etoglucid, fotemustine, hepsulfam, ifosfamide, improsulfan, irofulven, lomustine, mannosulfan, mechlorethamine, melphalan, mitobronitol, nedaplatin, nimustine, oxaliplatin, piposulfan, prednimustine, procarbazine, ranimustine, satraplatin, semustine, streptozocin, temozolomide, thiotepa, treosulfan, triaziquone, triethylenemelamine, triplatin tetranitrate, trofosphamide, and uramustine; an antibiotic, including, but not limiting to, aclarubicin, amrubicin, bleomycin, dactinomycin, daunorubicin, doxorubicin, elsamitrucin, epirubicin, idarubicin, menogaril, mitomycin, neocarzinostatin, pentostatin, pirarubicin, plicamycin, vairubicin, and zorubicin; an antimetabolite, including, but not limiting to, aminopterin, azacitidine, azathioprine, capecitabine, cladribine, clofarabine, cytarabine, decitabine, floxuridine, fludarabine, 5-fluorouracil, gemcitabine, hydroxyurea, mercaptopurine, methotrexate, nelarabine, pemetrexed, raltitrexed, tegafur-uracil, thioguanine, trimethoprim, trimetrexate, and vidarabine; an immunotherapy, an antibody therapy, including, but not limiting to, alemtuzumab, bevacizumab, cetuximab, galiximab, gemtuzumab, panitumumab, pertuzumab, rituximab, brentuximab, tositumomab, trastuzumab, 90 Y ibritumomab tiuxetan, ipilimumab, tremelimumab and anti-CTLA-4 antibodies; a hormone or hormone antagonist, including, but not limiting to, anastrozole, androgens, buserelin, diethylstilbestrol, exemestane, flutamide, fulvestrant, goserelin, idoxifene, letrozole, leuprolide, magestrol, raloxifene, tamoxifen, and toremifene; a taxane, including, but not limiting to, DJ-927, docetaxel, TPI 287, larotaxel, ortataxel, paclitaxel, DHA-paclitaxel, and tesetaxel; a retinoid, including, but not limiting to, alitretinoin, bexarotene, fenretinide, isotretinoin, and tretinoin; an alkaloid, including, but not limiting to, demecolcine, homoharringtonine, vinblastine, vincristine, vindesine, vinflunine, and vinorelbine; an antiangiogenic agent, including, but not limiting to, AE-941 (GW786034, Neovastat), ABT-510, 2-methoxyestradiol, lenalidomide, and thalidomide; a topoisomerase inhibitor, including, but not limiting to, amsacrine, belotecan, edotecarin, etoposide, etoposide phosphate, exatecan, irinotecan (also active metabolite SN-38 (7-ethyl-10-hydroxy-camptothecin)), lucanthone, mitoxantrone, pixantrone, rubitecan, teniposide, topotecan, and 9-aminocamptothecin; a kinase inhibitor, including, but not liming to, axitinib (AG 013736), dasatinib (BMS 354825), erlotinib, gefitinib, flavopiridol, imatinib mesylate, lapatinib, motesanib diphosphate (AMG 706), nilotinib (AMN107), seliciclib, sorafenib, sunitinib malate, AEE-788, BMS-599626, UCN-01 (7-hydroxystaurosporine), vemurafenib, dabrafenib, selumetinib, paradox breakers (such as PLX8394 or PLX7904), LGX818, BGB-283, pexidartinib (PLX3397) and vatalanib; a targeted signal transduction inhibitor including, but not limiting to bortezomib, geldanamycin, and rapamycin; a biological response modifier, including, but not limiting to, imiquimod, interferon-α, and interleukin-2; and other chemotherapeutics, including, but not limiting to 3-AP (3-amino-2-carboxyaldehyde thiosemicarbazone), altrasentan, aminoglutethimide, anagrelide, asparaginase, bryostatin-1, cilengitide, elesclomol, eribulin mesylate (E7389), ixabepilone, lonidamine, masoprocol, mitoguanazone, oblimersen, sulindac, testolactone, tiazofurin, mTOR inhibitors (e.g. sirolimus, temsirolimus, everolimus, deforolimus, INK28, AZD8055, PI3K inhibitors (e.g. BEZ235, GDC-0941, XL147, XL765, BMK120), cyclin dependent kinase (CDK) inhibitors (e.g., a CDK4 inhibitor or a CDK6 inhibitor, such as Palbociclib (PD-0332991), Ribocyclib (LEE011), Abemaciclib (LY2835219), P1446A-05, Abemaciclib (LY2835219), Trilaciclib (G1T28), etc.), AKT inhibitors, Hsp90 inhibitors (e.g. geldanamycin, radicicol, tanespimycin), farnesyltransferase inhibitors (e.g. tipifarnib), Aromatase inhibitors (anastrozole letrozole exemestane); an MEK inhibitor including, but are not limited to, AS703026, AZD6244 (Selumetinib), AZD8330, BIX 02188, CI-1040 (PD184352), GSK1120212 (also known as trametinib or JTP-74057), cobimetinib, PD0325901, PD318088, PD98059, RDEA119 (BAY 869766), TAK-733 and U0126-EtOH; tyrosine kinase inhibitors, including, but are not limited to, AEE788, AG-1478 (Tyrphostin AG-1478), AG-490, Apatinib (YN968D1), AV-412, AV-951 (Tivozanib), Axitinib, AZD8931, BIBF1120 (Vargatef), BIBW2992 (Afatinib), BMS794833, BMS-599626, Brivanib (BMS-540215), Brivanib alaninate (BMS-582664), Cediranib (AZD2171), Chrysophanic acid (Chrysophanol), Crenolanib (CP-868569), CUDC-101, CYC116, Dovitinih Dilactic acid (TK1258 Dilactic acid), E7080, Erlotinib Hydrochloride (Tarceva, CP-358774, OSI-774, NSC-718781), Foretinib (GSK1363089, XL880), Gefitinib (ZD-1839 or Iressa), Imatinib (Gleevec), Imatinib Mesylate, Ki8751, KRN 633, Lapatinib (Tykerb), Linifanib (ABT-869), Masitinib (Masivet, AB1010), MGCD-265, Motesanib (AMG-706), MP-470, Mubritinib (TAK 165), Neratinib (HKI-272), NVP-BHG712, OSI-420 (Desmethyl Erlotinib, CP-473420), OSI-930, Pazopanib HCl, PD-153035 HCi, PD173074, Pelitinib (EKB-569), PF299804, Ponatinib (AP24534), PP121, RAF265 (CHIR-265), Raf265 derivative, Regorafenib (BAY 73-4506), Sorafenib Tosylate (Nexavar), Sunitinib Malate (Sutent), Telatinib (BAY 57-9352), TSU-68 (SU6668), Vandetanib (Zactima), Vatalanib dihydrochloride (PTK787), WZ3146, WZ4002, WZ8040, quizartinib, Cabozantinib, XL647, EGFR siRNA, FLT4 siRNA, KDR siRNA, Antidiabetic agents such as metformin, PPAR agonists (rosiglitazone, pioglitazone, bezafibrate, ciprofibrate, clofibrate, gemfibrozil, fenofibrate, indeglitazar), DPP4 inhibitors (sitagliptin, vildagliptin, saxagliptin, dutogliptin, gemigliptin, alogliptin) or an EGFR inhibitor, including, but not limited to, AEE-788, AP-26113, BIBW-2992 (Tovok), CI-1033, GW-572016, Iressa, LY2874455, RO-5323441, Tarceva (Erlotinib, OSI-774), CUDC-101 and WZ4002.

In certain embodiments, the second chemotherapeutic agent is selected from afatinib, afuresertib, alectinib, alisertib, alvocidib, amsacrine, amonafide, amuvatinib, axitinib, azacitidine, azathioprine, bafetinib, barasertib, bendamustine, bleomycin, bosutinib, bortezomib, busulfan, cabozantinib, camptothecin, canertinib, capecitabine, cabazitaxel, carboplatin, carmustine, cenisertib, ceritinib, chlorambucil, cisplatin, cladribine, clofarabine, crenolanib, crizotinib, cyclophosphanide, cytarabine, dabrafenib, dacarbazine, dacomitinib, dactinomycin, danusertib, dasatinib, daunorubicin, decitabine, dinaciclib, docetaxel, dovitinib, doxorubicin, epirubicin, epitinib, eribulin mesylate, erriotinib, etirinotecan, etoposide, everolimus, exemestane, floxuridine, fludarabine, fluorouracil, gefitinib, gemcitabine, hydroxyurea, ibrutinib, icotinib, idarubicin, idelalisib, ifosfamide, imatinib, imetelstat, ipatasertib, irinotecan, ixabepilone, lapatinib, lenalidomide, lestaurtinib, lomustine, lucitanib, masitinib, mechlorethamine, melphalan, mercaptopurine, methotrexate, midostaurin, mitomycin, mitoxantrone, mubritinib, nelarabine, neratinib, nilotinib, nintedanib, omacetaxine mepesuccinate, olaparib, orantinib, oxaliplatin, paclitaxel, palbociclib, palifosfamide tris, pazopanib, pelitinib, pemetrexed, pentostatin, plicamycin, ponatinib, poziotinib, pralatrexate, procarbazine, quizartinib, raltitrexed, regorafenib, ruxolitinib, seliciclib, sorafenib, streptozocin, sulfatinib, sunitinib, tamoxifen, tandutinib, temozolomide, temsirolimus, teniposide, theliatinib, thioguanine, thiotepa, topotecan, uramustine, valrubicin, vandetanib, vemurafenib (Zelboraf), vincristine, vinblastine, vinorelbine, vindesine, and the like. In certain embodiments, the compounds herein are administered prior to, concurrently with, or subsequent to the treatment with the chemotherapeutic agent.

In certain embodiments, the method of treating a cancer comprises administering a therapeutically effective amount of a compound described herein and a therapeutically effective amount a biologic agent used to treat cancer. In certain embodiments, the biologic agent is selected from anti-BAFF (e.g., belimumab); anti-CCR4 (e.g., mogamulizumab); anti-CD19/CD3 (e.g., blinatumomab); anti-CD20 (e.g., obinutuzumab, rituximab, ibritumomab tiuxetan, ofatumumab, tositumomab); anti-CD22 (e.g., moxetumomab pasudotox); anti-CD30 (e.g., brentuximab vedotin); anti-CD33 (e.g., gemtuzumab); anti-CD37 (e.g., otlertuzumab); anti-CD38 (e.g., daratumumab); anti-CD52 (e.g., alemtuzumab); anti-CD56 (e.g., lorvotuzumab mertansine); anti-CD74 (e.g., milatuzumab); anti-CD105; anti-CD248 (TEM1) (e.g., ontuxizumab); anti-CTLA4 (e.g., tremelimumab, ipilimumab); anti-EGFL7 (e.g., parsatuzumab); anti-EGFR (HER1/ERBB1) (e.g., panitumumab, nimotuzumab, necitumumab, cetuximab, imgatuzumab, futuximab); anti-FZD7 (e.g., vantictumab); anti-HER2 (ERBB2/neu) (e.g., margetuximab, pertuzumab, ado-trastuzumab emtansine, trastuzumab); anti-HER3 (ERBB3); anti-HGF (e.g., rilotumumab, ficlatuzumab); anti-IGF-1R (e.g., ganitumab, figitumumab, cixutumumab, dalotuzumab); anti-IGF-2R; anti-KIR (e.g., lirilumab, onartuzumab); anti-MMP9; anti-PD-1 (e.g., nivolumab, pidilizumab, lambrolizumab); anti-PD-L1 (e.g. Atezolizumab); anti-PDGFRa (e.g., ramucirumab, tovetumab); anti-PD-L2; anti-PIGF (e.g., ziv-aflibercept); anti-RANKL (e.g., denosumab); anti-TNFRSF 9 (CD 137/4-1 BB) (e.g., urelumab); anti-TRAIL-RI/DR4,R²/D5 (e.g., dulanermin); anti-TRAIL-R¹/D4 (e.g., mapatumumab); anti-TRAIL-R²/D5 (e.g., conatumumab, lexatumumab, apomab); anti-VEGFA (e.g., bevacizumab, ziv-aflibercept); anti-VEGFB (e.g., ziv-aflibercept); and anti-VEGFR2 (e.g., ramucirumab).

### Effective Amount and Dosing

In certain embodiments, a pharmaceutical composition of the compound is administered to a subject, preferably a human, at a therapeutically effective dose to prevent, treat, or control a condition or disease as described herein. The pharmaceutical composition is administered to a subject in an amount sufficient to elicit an effective therapeutic response in the subject. An effective therapeutic response is a response that at least partially arrests or slows the symptoms or complications of the condition or disease. An amount adequate to accomplish this is defined as “therapeutically effective dose” or “therapeutically effective amount.” The dosage of compounds can take into consideration, among others, the species of warm-blooded animal (mammal), the body weight, age, condition being treated, the severity of the condition being treated, the form of administration, route of administration. The size of the dose also will be determined by the existence, nature, and extent of any adverse effects that accompany the administration of a particular therapeutic compound in a particular subject.

In certain embodiments, the compounds can be administered with one or more of a second compound, sequentially or concurrently, either by the same route or by different routes of administration. When administered sequentially, the time between administrations is selected to benefit, among others, the therapeutic efficacy and/or safety of the combination treatment. In certain embodiments, the compounds herein can be administered first followed by a second compound, or alternatively, the second compound administered first followed by the compounds of the present disclosure.

When administered concurrently, the compound can be administered separately at the same time as the second compound, by the same or different routes, or administered in a single composition by the same route. In certain embodiments, the amount and frequency of administration of the second compound can used standard dosages and standard administration frequencies used for the particular compound. See, e.g., Physicians' Desk Reference, 70th Ed., PDR Network, 2015; incorporated herein by reference.

It to be understood that optimum dosages, toxicity, and therapeutic efficacy of such compounds may vary depending on the relative potency of individual compound and can be determined by standard pharmaceutical procedures in cell cultures or experimental animals, for example, by determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio, LD₅₀/ED₅₀. While certain agents that exhibit toxic side effects can be used, care should be used to design a delivery system that targets such agents to the site of affected tissue to minimize potential damage to normal cells and, thereby, reduce side effects.

The data obtained from, for example, cell culture assays and animal studies can be used to formulate a dosage range for use in humans. The dosage of such small molecule compounds lies preferably within a range of circulating concentrations that include the ED₅₀ with little or no toxicity. The dosage can vary within this range depending upon the dosage form employed and the route of administration. For any compounds used in the methods disclosed herein, the therapeutically effective dose can be estimated initially from cell culture assays. A dose can be formulated in animal models to achieve a circulating plasma concentration range that includes the IC₅₀ (the concentration of the test compound that achieves a half-maximal inhibition of symptoms) as determined in cell culture. Such information can be used to more accurately determine useful doses in humans. Levels in plasma can be measured, for example, by high performance liquid chromatography (HPLC).

### Methods of Preparation

The following examples are provided to further illustrate the methods of the present disclosure, and the compounds and compositions for use in the methods. The examples described are illustrative only and are not intended to limit the scope of the invention(s) in any way. The disclosures of all articles and references mentioned in this application, including patents, are incorporated herein by reference in their entirety.

The compounds of the present disclosure can be synthesized in view of the guidance provided herein, incorporating known chemical reactions and related procedures such as separation and purification. Representative methods and procedures for preparation of the compounds in this disclosure are described below and in the Examples. Acronyms are abbreviations are used per convention which can be found in literature and scientific journals.

In certain embodiments, provided is a process for preparing a compound of Formula I, or a tautomer, stereoisomer, mixture of stereoisomers, isotopically enriched analog, or salt thereof:

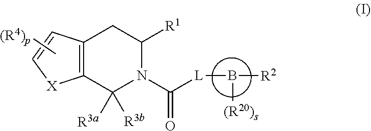

wherein each of ring B, L, X, R¹, R², R^{3a}, R^{3b}, R⁴, R²⁰, p, and s are independently as defined herein, comprising contacting a compound of Formula 1-5 with a compound of Formula 1-6:

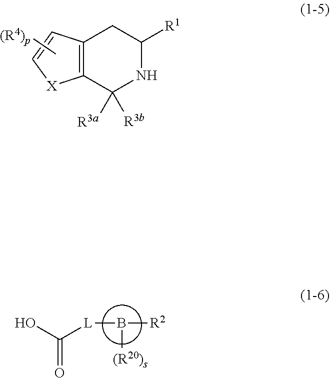

under reaction conditions sufficient to provide the compound of Formula I, or a tautomer, stereoisomer, mixture of stereoisomers, isotopically enriched analog, or salt thereof.

In certain embodiments, provided is a process for preparing a compound of Formula I-a, or a tautomer, stereoisomer, mixture of stereoisomers, isotopically enriched analog, or salt thereof:

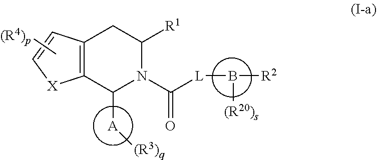

wherein each of ring A, ring B, L, X, R¹, R², R³, R⁴, R²⁰, p, q, and s are independently as defined herein, comprising contacting a compound of Formula 1-a-5 with a compound of Formula 1-6:

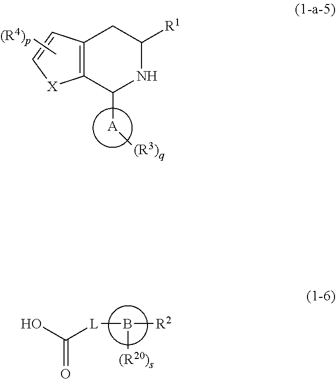

under reaction conditions sufficient to provide the compound of Formula I, or a tautomer, stereoisomer, mixture of stereoisomers, isotopically enriched analog, or salt thereof.

In certain embodiments, provided is a process for preparing a compound of Formula 1-a-5, or a tautomer, stereoisomer, mixture of stereoisomers, isotopically enriched analog, or salt thereof, wherein each of ring A, X, R¹, R³, R⁴, p, and q are independently as defined herein, comprising cyclizing a compound of Formula 1-a-3:

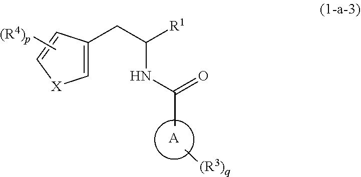

under reaction conditions sufficient to provide the compound of Formula 1-a-5, or a tautomer, stereoisomer, mixture of stereoisomers, isotopically enriched analog, or salt thereof.

In certain embodiments, provided is a process for preparing a compound of Formula II, or a tautomer, stereoisomer, mixture of stereoisomers, isotopically enriched analog, or salt thereof:

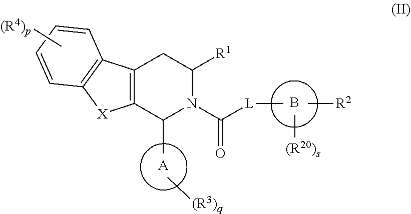

wherein each of ring A, ring B, L, X, R¹, R², R³, R⁴, R²⁰, p, q, and s are independently as defined herein, comprising contacting a compound of Formula 3-5 with a compound of Formula 3-6:

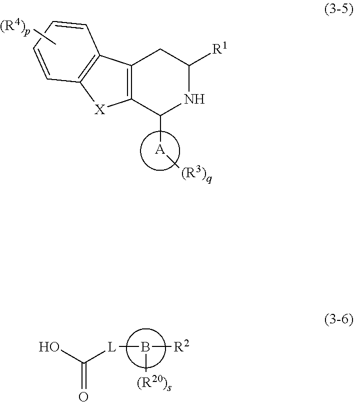

under reaction conditions sufficient to provide the compound of Formula II, or a tautomer, stereoisomer, mixture of stereoisomers, isotopically enriched analog, or salt thereof.

In certain embodiments, provided is a process for preparing a compound of Formula 3-5, or a tautomer, stereoisomer, mixture of stereoisomers, isotopically enriched analog, or salt thereof, wherein each of ring A, X, R¹, R³, R⁴, p, and q are independently as defined herein, comprising cyclizing a compound of Formula 1-3:

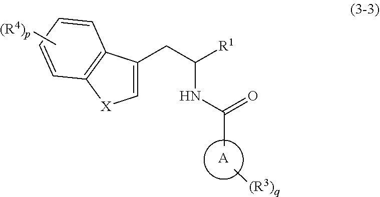

under reaction conditions sufficient to provide the compound of Formula 3-5, or a tautomer, stereoisomer, mixture of stereoisomers, isotopically enriched analog, or salt thereof.

It is understood that the starting materials and reaction conditions may be varied, the sequence of the reactions altered, and additional steps employed to produce compounds encompassed by the present disclosure, as demonstrated by the following examples. General references for known chemical reactions useful for synthesizing the disclosed compounds are available (see, e.g., Smith and March, March's Advanced Organic Chemistry: Reactions, Mechanisms, and Structure, Fifth Edition, Wiley Interscience, 2001; or Carey and Sundberg, Advanced Organic Chemistry, Part B. Reaction and Synthesis; Fifth Edition, Springer, 2007; or Li, J. J. Name Reactions, A Collection of Detailed Mechanisms and Synthetic Applications; Fifth Edition, Springer, 2014).

It will be appreciated that where typical or specific process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in Wuts, P. G. M., Greene, T. W., & Greene, T. W. (2006). Greene's protective groups in organic synthesis. Hoboken, N.J., Wiley-Interscience, and references cited therein.

Furthermore, the compounds of this disclosure may contain one or more chiral centers. Accordingly, if desired, such compounds can be prepared or isolated as pure stereoisomers, i.e., as individual enantiomers or diastereomers or as stereoisomer-enriched mixtures. All such stereoisomers (and enriched mixtures) are included within the scope of this disclosure, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents, and the like.

The starting materials for the following reactions are generally known compounds or can be prepared by known procedures or obvious modifications thereof. For example, many of the starting materials are available from commercial suppliers such as Aldrich Chemical Co. (Milwaukee, Wisconsin, USA), Bachem (Torrance, California, USA), Emnka-Chemce or Sigma (St. Louis, Missouri, USA). Others may be prepared by procedures or obvious modifications thereof, described in standard reference texts such as Fieser and Fieser's Reagents for Organic Synthesis, Volumes 1-15 (John Wiley, and Sons. 1991), Rodd's Chemistry of Carbon Compounds, Volumes 1-5, and Supplementals (Elsevier Science Publishers, 1989) organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989).

### General Synthesis

In certain embodiments, compounds disclosed herein can be according to the general schemes shown below. For example, compounds of Formula I can be prepared according to the general syntheses outlined below in Scheme 1, where suitable reagents can be purchased form commercial sources or synthesized via known methods or methods adapted from the examples provided herein. In Scheme 1, each of ring B, L, X, R¹, R², R³, R^{3b}, R⁴, R²⁰, p, and s are independently as defined herein.

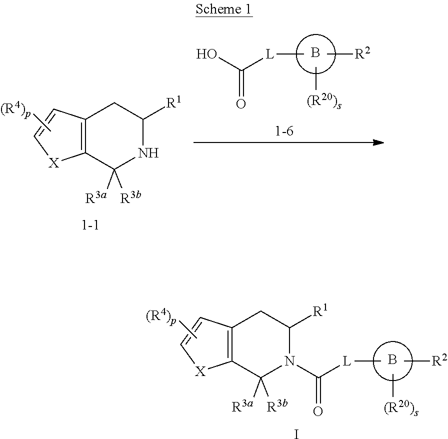

In Scheme 1, compounds of Formula I can be provided by coupling compound 1-1 with compound 1-6 under amide bond forming reaction conditions suitable to provide compounds of Formula I. Upon each reaction completion, each of the intermediate or final compounds can be recovered, and optionally purified, by conventional techniques such as neutralization, extraction, precipitation, chromatography, filtration, and the like.

Compounds of Formula I-a can be prepared according to the general syntheses outlined below in Scheme 1-a, where suitable reagents can be purchased form commercial sources or synthesized via known methods or methods adapted from the examples provided herein. In Scheme 1-a, each of ring A, ring B, L, X, R¹, R², R³, R⁴, R²⁰, p, q, and s are independently as defined herein.

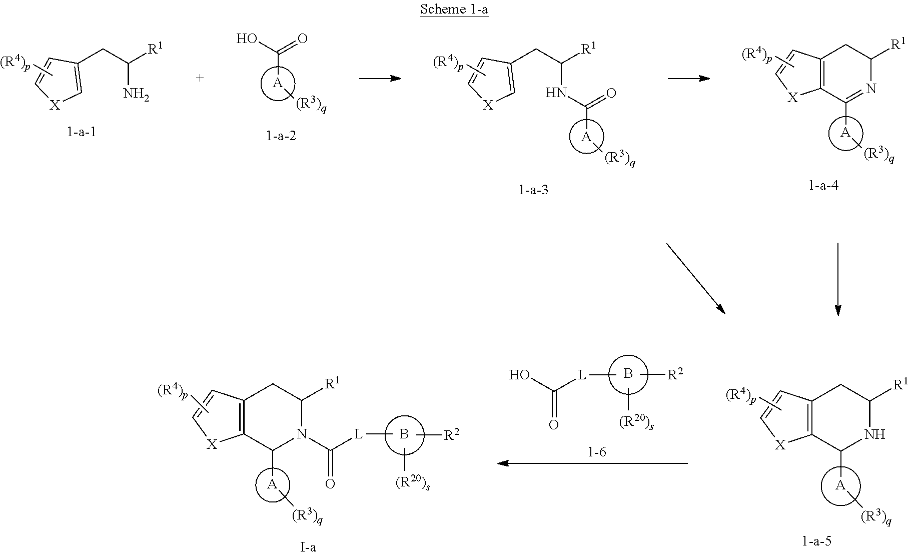

In Scheme 1-a, compound 1-a-3 can be provided by coupling amine 1-a-1 with acid 1-a-2 under standard amide bond forming reaction conditions. Cyclization of compound 1-a-3 to provide compound 1-a-5 can be achieved by first forming compound 1-a-4 followed by reduction using a hydride (e.g., NaBH₄, LiAlH₄, etc.). Alternatively, compound 1-a-5 can be provided directly from compound 1-a-3 under suitable conditions, such as an aprotic solvent in the presence of an acid catalyst. Compounds of Formula I-a can then be provided by coupling compound 1-a-5 with compound 1-6 under reaction conditions suitable to provide compounds of Formula I-a. Upon each reaction completion, each of the intermediate or final compounds can be recovered, and optionally purified, by conventional techniques such as neutralization, extraction, precipitation, chromatography, filtration, and the like.

Appropriate starting materials and reagents for use in Scheme 1 or Scheme 1-a can be purchased or prepared by methods known to one of skill in the art. As shown in Scheme 2, chiral or enantiomerically enriched starting materials can be provided for use in the method of Scheme 1 or in the preparation of compound 1-1 of Scheme 1 by converting a chiral or enantiomerically enriched amino alcohol to an oxathiazolidine dioxide 2-2. In Scheme 2, X, R¹, R⁴, and p are independently as defined herein, M is a metal halide (e.g., MgBr), and PG is a protecting group (e.g., Boc).

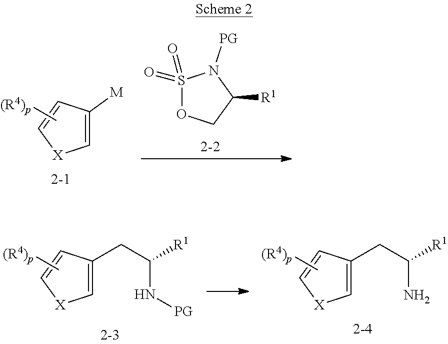

Referring to Scheme 2, compound 2-1 is coupled to compound 2-2 under standard coupling conditions to produce compound 2-3. The reaction is typically conducted in the presence of suitable catalyst (e.g., CuI) using suitable solvents/solvent mixtures. Deprotection of compound 2-3 provides compound 2-4. Upon reaction completion, each intermediate can be recovered by conventional techniques such as neutralization, extraction, precipitation, chromatography, filtration, and the like.

In some embodiments of the methods of Scheme 1, Scheme 1-a, and Scheme 2, the various substituents on the starting compound (e.g., compound 1-1 and compound 1-2, (e.g., ring A, R¹, R², R³, etc.)) are as defined for Formula I or Formula I-a. However, it should also be appreciated that chemical derivatization and/or functional group interconversion, can be used to further modify of any of the compounds of Scheme 1, Scheme 1-a, or Scheme 2 in order to provide the various compounds of Formula I or Formula I-a.

In some embodiments, compounds according to Formula II can be prepared according to the general syntheses outlined below in Scheme 3, where suitable reagents can be purchased form commercial sources or synthesized via known methods or methods adapted from the examples provided herein. In Scheme 3, each of ring A, ring B, L, X, R¹, R², R³, R⁴, R²⁰, p, q, and s are independently as defined herein.

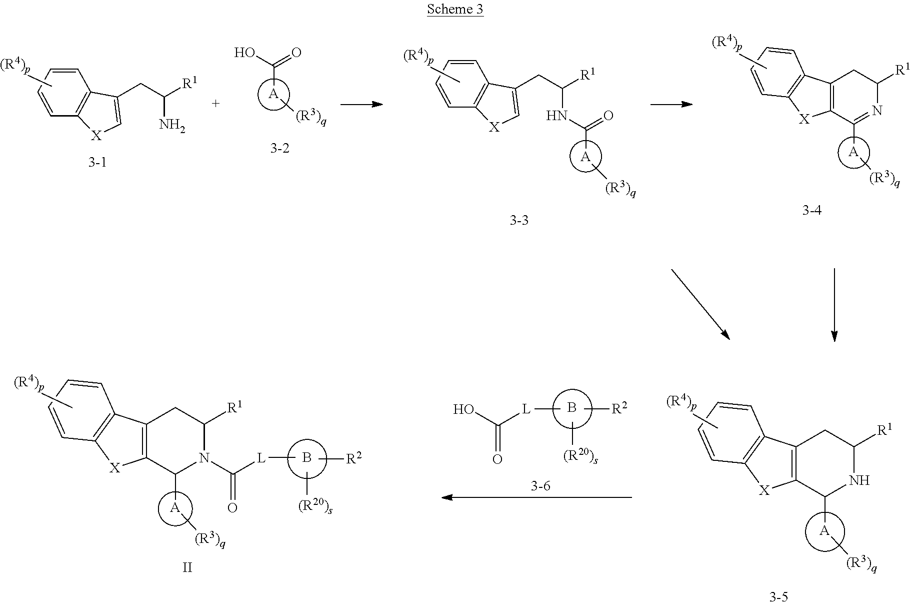

In Scheme 3, compound 3-3 can be provided by coupling amine 3-1 with acid 3-2 under standard amide bond forming reaction conditions. Cyclization of compound 3-3 to provide compound 3-5 can be achieved by first forming compound 3-4 followed by reduction using a hydride (e.g., NaBH₄, LiAlH₄, etc.). Alternatively, compound 3-5 can be provided directly from compound 3-3 under suitable conditions, such as an aprotic solvent in the presence of an acid catalyst. Compounds of Formula I can then be provided by coupling compound 3-5 with compound 3-6 under reaction conditions suitable to provide compounds of Formula II. Upon each reaction completion, each of the intermediate or final compounds can be recovered, and optionally purified, by conventional techniques such as neutralization, extraction, precipitation, chromatography, filtration, and the like.

Appropriate starting materials and reagents for use in Scheme 3 can be purchased or prepared by methods known to one of skill in the art. As shown in Scheme 4, chiral or enantiomerically enriched starting materials can be provided for use in the method of Scheme 3 by converting a chiral or enantiomerically enriched amino alcohol to an oxathiazolidine dioxide 4-2. In Scheme 4, X, R¹, R⁴, and p are independently as defined herein, M is a metal halide (e.g., MgBr), and PG is a protecting group (e.g., Boc).

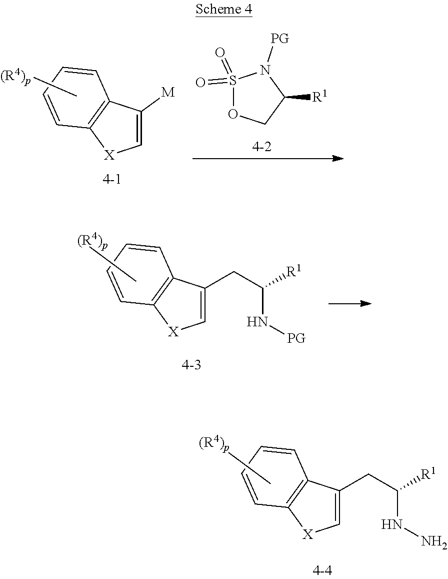

Referring to Scheme 4, compound 4-1 is coupled to compound 4-2 under standard coupling conditions to produce compound 4-3. The reaction is typically conducted in the presence of suitable catalyst (e.g., CuI) using suitable solvents/solvent mixtures. Deprotection of compound 2-3 provides compound 4-4. Upon reaction completion, each intermediate can be recovered by conventional techniques such as neutralization, extraction, precipitation, chromatography, filtration, and the like.

In some embodiments of the methods of Scheme 3 and Scheme 4, the various substituents on the starting compound (e.g., compound 3-1 and compound 3-2, (e.g., ring A, R¹, R², R³, etc.)) are as defined for Formula I. However, it should also be appreciated that chemical derivatization and/or functional group interconversion, can be used to further modify of any of the compounds of Scheme 3 or Scheme 4 in order to provide the various compounds of Formula II.

Other compounds of the disclosure can be synthesized using the synthetic routes above and adapting chemical synthetic procedures available to the skilled artisan. Exemplary methods of synthesis are provided in the Examples. It is to be understood that each of the procedures describing synthesis of exemplary compounds are part of the specification, and thus incorporated herein into the Detailed Description of this disclosure. Related synthetic schema and examples are included in International Patent Applications Nos. PCT/US2019/019854, filed Feb. 27, 2019, and PCT/US2020/020150, filed Feb. 27, 2020, which are herein incorporated by reference in their entireties.

### Synthetic Examples

### Synthesis of Compound 1a—Version 1

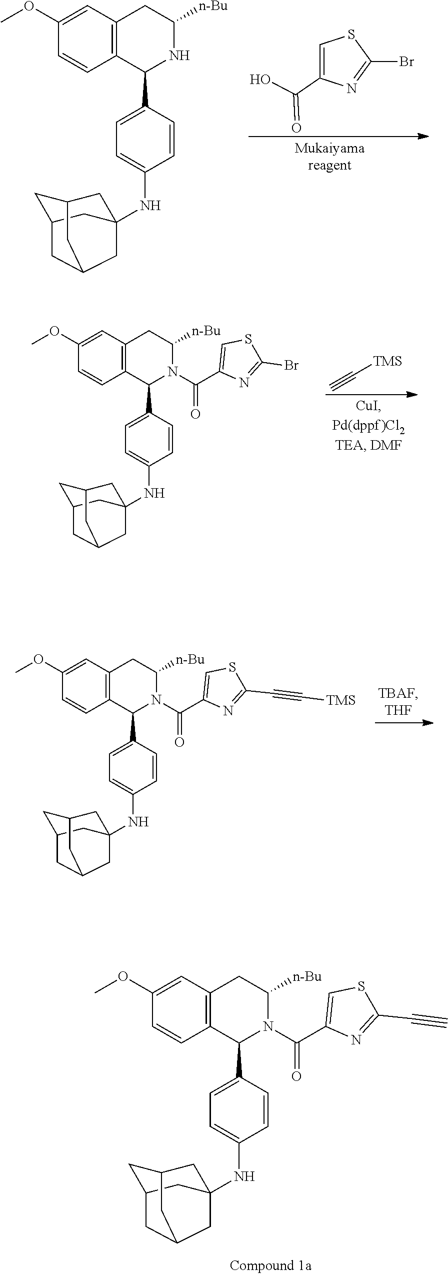

((1S,3S)-1-(4-(((3R,5R,7R)-adamantan-1-yl)amino)phenyl)-3-butyl-6-methoxy-3,4-dihydroisoquinolin-2(1H)-yl)(2-bromothiazol-4-yl)methanone: A solution of 2-bromothiazole-4-carboxylic acid (93.57 mg, 449.79 umol, 1 eq) in DCM (3 mL) was added 2-chloro-1-methyl-pyridin-1-ium;iodide (114.91 mg, 449.79 umol, 1 eq) at 25° C. The reaction was stirred at 25° C. for 0.5 h. Then it was added to the solution of (3R,5R,7R)—N-(4-((1S,3S)-3-butyl-6-methoxy-1,2,3,4-tetrahydroisoquinolin-1-yl)phenyl)adamantan-1-amine (200 mg, 449.79 umol, 1 eq) and Et₃N (91.03 mg, 899.58 umol, 125.21 uL, 2 eq) in DCM (3 mL) at 0° C. The reaction mixture was stirred at 0° C. for 1 h to give a yellow solution. LCMS showed a new peak give the desired MS. Water (50 mL) was added, the reaction was extracted with EtOAc (50 mL*2). The combined organic layers dried over Na₂SO₄, concentrated in vacuum to give ((1S,3S)-1-(4-(((3R,5R,7R)-adamantan-1-yl)amino)phenyl)-3-butyl-6-methoxy-3,4-dihydroisoquinolin-2(1H)-yl)(2-bromothiazol-4-yl)methanone (290 mg, crude) as yellow oil.

((1S,3S)-1-(4-(((3R,5R,7R)-adamantan-1-yl)amino)phenyl)-3-butyl-6-methoxy-3,4-dihydroisoquinolin-2(1H)-yl)(2-((trimethylsilyl)ethynyl)thiazol-4-yl)methanone: A 100 mL flask was charged with ((1S,3S)-1-(4-(((3R,5R,7R)-adamantan-1-yl)amino)phenyl)-3-butyl-6-methoxy-3,4-dihydroisoquinolin-2(1H)-yl)(2-bromothiazol-4-yl)methanone (250 mg, 393.91 umol, 1 eq), Pd(PPh₃)₂Cl₂ (27.65 mg, 39.39 umol, 0.1 eq), CuI (7.50 mg, 39.39 umol, 0.1 eq), ethynyl(trimethyl)silane (154.76 mg, 1.58 mmol, 218.27 uL, 4 eq), Et₃N (398.59 mg, 3.94 mmol, 548.27 uL, 10 eq) and THF (6 mL). The reaction was heated at 60° C. for 8 h under N₂ protection. LCMS showed a new peak give the desired MS. Water (50 mL) was added, the reaction was extracted with EtOAc (50 mL*2). The combined organic layers dried over Na₂SO₄, concentrated in vacuum to give ((1S,3S)-1-(4-(((3R,5R,7R)-adamantan-1-yl)amino)phenyl)-3-butyl-6-methoxy-3,4-dihydroisoquinolin-2(1H)-yl)(2-((trimethylsilyl)ethynyl)thiazol-4-yl)methanone (250 mg, crude) as black oil.

((3S)-1-(4-(((3R,5R,7R)-adamantan-1-yl)amino)phenyl)-3-butyl-6-methoxy-3,4-dihydroisoquinolin-2(1H)-yl)(2-ethynylthiazol-4-yl)methanone: A solution of ((1S,3S)-1-(4-(((3R,5R,7R)-adamantan-1-yl)amino)phenyl)-3-butyl-6-methoxy-3,4-dihydroisoquinolin-2(1H)-yl)(2-((trimethylsilyl)ethynyl)thiazol-4-yl)methanone (310 mg, 475.48 umol, 1 eq) in THF (15 mL) cooled to 0° C. was added TBAF (1 M in THF, 570.58 uL, 1.2 eq). The reaction was stirred at 0° C. for 1 h to give a black solution. LCMS showed a new peak give the desired MS. Water (100 mL) was added, the reaction was extracted with EtOAc (100 mL*2). The combined organic layers dried over Na₂SO₄, concentrated in vacuum to give a black oil. The crude was purified by flash column chromatography (SiO₂, PE to 20% PE in EA). The compound ((3S)-1-(4-(((3R,5R,7R)-adamantan-1-yl)amino)phenyl)-3-butyl-6-methoxy-3,4-dihydroisoquinolin-2(1H)-yl)(2-ethynylthiazol-4-yl)methanone (55.76 mg, 96.17 umol, 20.23% yield) was obtained as white solid. LC-MS (m/z): 580.6 [M+H]⁺; ¹H NMR (400 MHz, CHLOROFORM-d) δ=8.00 (br s, 0.39H), 7.45 (br s, 0.49H), 7.19 (br s, 1H), 7.09-6.96 (m, 1H), 6.86-6.73 (m, 2H), 6.68 (d, J=2.3 Hz, 1H), 6.66-6.46 (m, 3H), 5.40 (br s, 0.46H), 4.84 (br s, 0.52H), 3.79 (s, 3H), 3.49 (br s, 1H), 3.23-3.00 (m, 1H), 2.85-2.65 (m, 1H), 2.06 (br s, 3H), 1.78 (br s, 6H), 1.70-1.54 (m, 6H), 1.27-1.19 (m, 2H), 1.17-0.89 (m, 4H), 0.88-0.66 (m, 3H)

### Synthesis of Compound 1a—Version 2

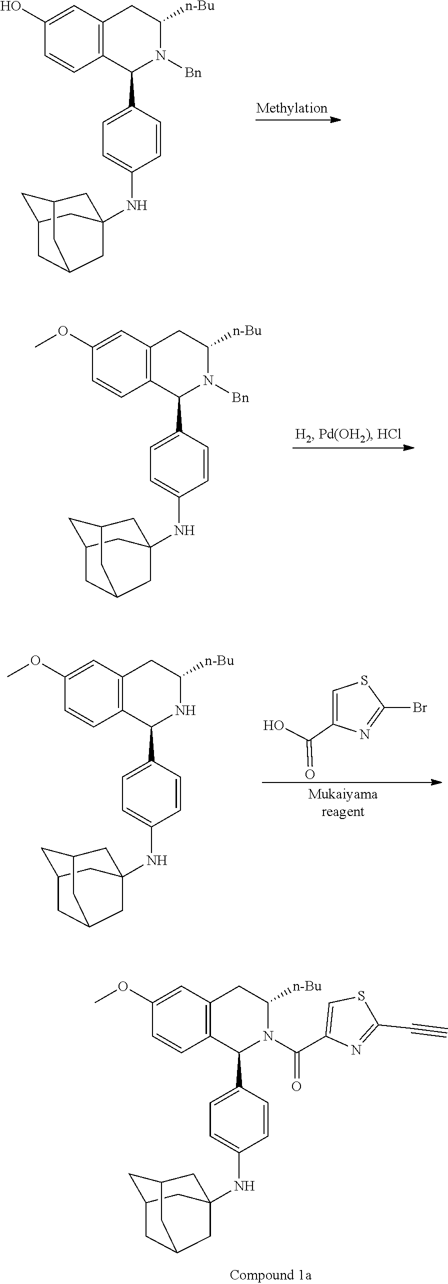

(3R,5R,7R)—N-(4-((1S,3S)-2-benzyl-3-butyl-6-methoxy-1,2,3,4-tetrahydroisoquinolin-1-yl)phenyl)adamantan-1-amine: To a solution of (1S,3S)-1-(4-(((3R,5R,7R)-adamantan-1-yl)amino)phenyl)-2-benzyl-3-butyl-1,2,3,4-tetrahydroisoquinolin-6-ol (4 g, 7.68 mmol, 1 eq) in DMF (40 mL) was added 50% NaOH·aq (1.23 g, 15.36 mmol, 50% purity, 2 eq) and Mel (1.09 g, 7.68 mmol, 478.19 uL. 1 eq) in DMF (4 mL) at 0-5° C. The reaction mixture was stirred at 0-5° C. for 30 min to give a yellow solution. LCMS showed the reaction was completed. The reaction mixture was poured into water (150 mL) and extracted with EtOAc (100 mL*3). The combined organic phases were washed with water (100 mL*3), sat. brine (100 mL), dried over Na₂SO₄, filtered and concentrated to dryness. The crude product on notebook page ES20635-283 was combined for further purification. The organic layers were concentrated under vacuum. The residue was purified by column chromatography (SiO₂, Petroleum ether/Ethyl acetate=100/0 to 90/10) to give (3R,5R,7R)—N-(4-((1S,3S)-2-benzyl-3-butyl-6-methoxy-1,2,3,4-tetrahydroisoquinolin-1-yl)phenyl)adamantan-1-amine (3.8 g) as colorless oil.

(3R,5R,7R)—N-(4-((1S,3S)-3-butyl-6-methoxy-1,2,3,4-tetrahydroisoquinolin-1-yl)phenyl)adamantan-1-amine: To a solution of (3R,5R,7R)—N-(4-((1S,3S)-2-benzyl-3-butyl-6-methoxy-1,2,3,4-tetrahydroisoquinolin-1-yl)phenyl)adamantan-1-amine (3.8 g, 7.11 mmol, 1 eq) in MeOH (100 mL) and HCl (12 M, 1.78 mL, 3 eq) was added Pd(OH)₂ (0.5 g, 7.11 mmol, 20% purity, 1 eq) at 25° C. The reaction mixture was purged with H₂ for 3 times and stirred at 25° C. under H₂ (15 Psi) for 16 hr to give a black suspension. The reaction mixture was filtered by a pad of celite and the filtrate was concentrated to dryness. The residue was diluted with EtOAc (80 mL) and washed with sat. NaHCO₃ aq. (80 mL×3), sat. brine (30 mL), dried over Na₂SO₄, filtered and concentrated to dryness. The residue was purified by column chromatography (SiO₂, Petroleum ether/Ethyl acetate=100/0 to 70/30) to give (3R,5R,7R)—N-(4-((1S,3S)-3-butyl-6-methoxy-1,2,3,4-tetrahydroisoquinolin-1-yl)phenyl)adamantan-1-amine (2.1 g, 4.72 mmol, 66.46% yield, N/A purity) as light yellow oil.

((1S,3S)-1-(4-(((3R,5R,7R)-adamantan-1-yl)amino)phenyl)-3-butyl-6-methoxy-3,4-dihydroisoquinolin-2(1H)-yl)(2-ethynylthiazol-4-yl)methanone: A solution of 2-ethynylthiazole-4-carboxylic acid (344.45 mg, 2.25 mmol, 1 eq) in DCM (10 mL) was added 2-chloro-1-methyl-pyridin-1-ium;iodide (574.57 mg, 2.25 mmol, 1 eq) at 25° C. The reaction was stirred at 25° C. for 0.5 hr. Then it was added to the solution of (3R,5R,7R)—N-(4-((1S,3S)-3-butyl-6-methoxy-1,2,3,4-tetrahydroisoquinolin-1-yl)phenyl)adamantan-1-amine (1 g, 2.25 mmol, 1 eq) and TEA (455.14 mg, 4.50 mmol, 626.05 uL, 2 eq) in DCM (10 mL) 0° C. The reaction mixture was stirred at 0° C. for 2 hr to give a yellow solution. The crude reaction mixture on notebook page ES20635-286 was combined for workup. The reaction was quenched with saturated sat. NH₄Cl aq. (20 mL) and extracted with DCM (20 mL*3). The combined organic layers were washed with brine (10 mL) and dried over anhydrous sodium sulfate, filtered and concentrated under reduced pressure. The crude product was purified by flash column (SiO₂, Petroleum ether/Ethyl acetate=100/0 to 70/30) to give ((1S,3S)-1-(4-(((3R,5R,7R)-adamantan-1-yl)amino)phenyl)-3-butyl-6-methoxy-3,4-dihydroisoquinolin-2(1H)-yl)(2-ethynylthiazol-4-yl)methanone (935.2 mg, 1.59 mmol, 70.72% yield, 98.6% purity) as white solid. LC-MS (m/z): 580.2 [M+H]⁺; ¹H NMR (400 MHz, CHLOROFORM-d) δ ppm 8.00 (br s, 1H) 7.45 (br s, 1H) 7.19 (br s, 1H) 7.04 (br s, 1H) 6.87-6.73 (m, 2H) 6.68 (d, J=2.26 Hz, 1H) 6.65-6.46 (m, 2H) 6.64-6.46 (m, 1H) 5.49-4.74 (m, 1H) 3.79 (s, 3H) 3.49 (br s, 1H) 3.24-2.98 (m, 1H) 2.85-2.61 (m, 1H) 2.06 (br s, 3H) 1.84-1.52 (m, 14H) 1.41-0.94 (m, 6H) 0.92-0.59 (m, 3H)

### Synthesis of Compound 2a

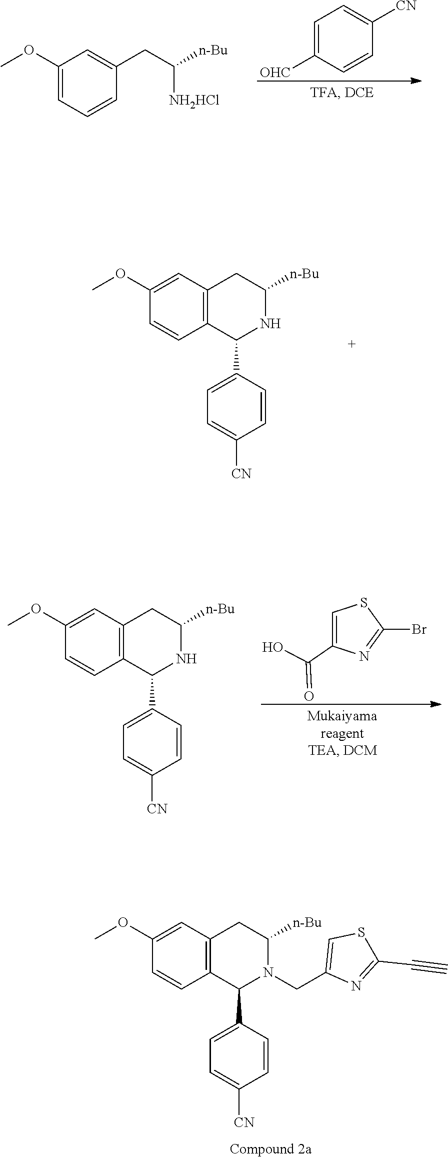

4-((1S,3S)-3-butyl-6-methoxy-1,2,3,4-tetrahydroisoquinolin-1-yl)benzonitrile: To a stirred solution of (S)-1-(3-methoxyphenyl)hexan-2-amine (650 mg, 3.14 mmol, 1 eq) in DCE (20 mL) was added 4-formylbenzonitrile (411.15 mg, 3.14 mmol, 1 eq) and TFA (715.01 mg, 6.27 mmol, 464.29 uL, 2 eq) at 20° C. The mixture was stirred at 80° C. for 4 hr. LCMS showed the reaction was completed. The solution was basified by NaHCO₃ to pH=7.5. Then the reaction was extracted by DCE (30 ml*2) and concentrated to give the crude product. The mixture was purified by flash column (SiO₂, DCE:McOH=1:0 to 9:1) and prep-TLC(SiO₂, DCE:MeOH=10:1) to give the 4-((1S,3S)-3-butyl-6-methoxy-1,2,3,4-tetrahydroisoquinolin-1-yl)benzonitrile (more polarity spot) (200 mg, 624.17 umol, 19.91% yield) as yellow oil and 4-((1R,3S)-3-butyl-6-methoxy-1,2,3,4-tetrahydroisoquinolin-1-yl)benzonitrile (less polarity spot) (200 mg, 624.17 umol, 19.91% yield) as yellow oil.

4-((1S,3S)-3-butyl-2-(2-ethynylthiazole-4-carbonyl)-6-methoxy-1,2,3,4-tetrahydroisoquinolin-1-yl)benzonitrile: To a solution of 4-((1S,3S)-3-butyl-6-methoxy-1,2,3,4-tetrahydroisoquinolin-1-yl)benzonitrile (65 mg, 202.85 umol, 1 eq) in DCE (5 mL) was added TEA (41.05 mg, 405.71 umol, 56.47 uL, 2 eq). The mixture was stirred at 20° C. for 0.1 h. Then 2-ethynylthiazole-4-carboxylic acid (31.07 mg, 202.85 umol, 1 eq) and 2-chloro-1-methyl-pyridin-1-ium;iodide (62.19 mg, 243.43 umol, 1.2 eq) was added at 20° C. The mixture was stirred at 20° C. for 1 h to give a yellow solution. LCMS showed the desired MS. The mixture was washed by H₂O (150 ml) and extracted by DCM (100 ml*2), then the mixture was concentrated to give the crude product. The mixture was purified by prep-HPLC (Welch Xtimate C18 100*40 mm*3 um; mobile phase: [water(HCl)-ACN]; B %: 60%-90%, 8 min) to give 4-((1S,3S)-3-butyl-2-(2-ethynylthiazole-4-carbonyl)-6-methoxy-1,2,3,4-tetrahydroisoquinolin-1-yl)benzonitrile (14.67 mg, 32.20 umol, 15.87% yield) as brown solid. LC-MS (m/z): 456.0[M+Na]⁺; ¹H NMR (400 MHz, CHLOROFORM-d) 6=8.06-7.68 (m, 1H), 7.58-7.38 (m, 3H), 7.23 (br d, J=8.5 Hz, 1H), 7.09 (br s, 1H), 6.82-6.76 (m, 1H), 6.70 (br s, 1H), 6.40 (s, 1H), 5.50 (br s, 1H), 3.79 (s, 3H), 3.60-3.47 (m, 1H), 3.23 (br dd, J=3.9, 15.3 Hz, 1H), 3.01-2.70 (m, 1H), 1.37-0.96 (m, 7H), 0.90-0.64 (m, 3H)

### Synthesis of Compound 4a

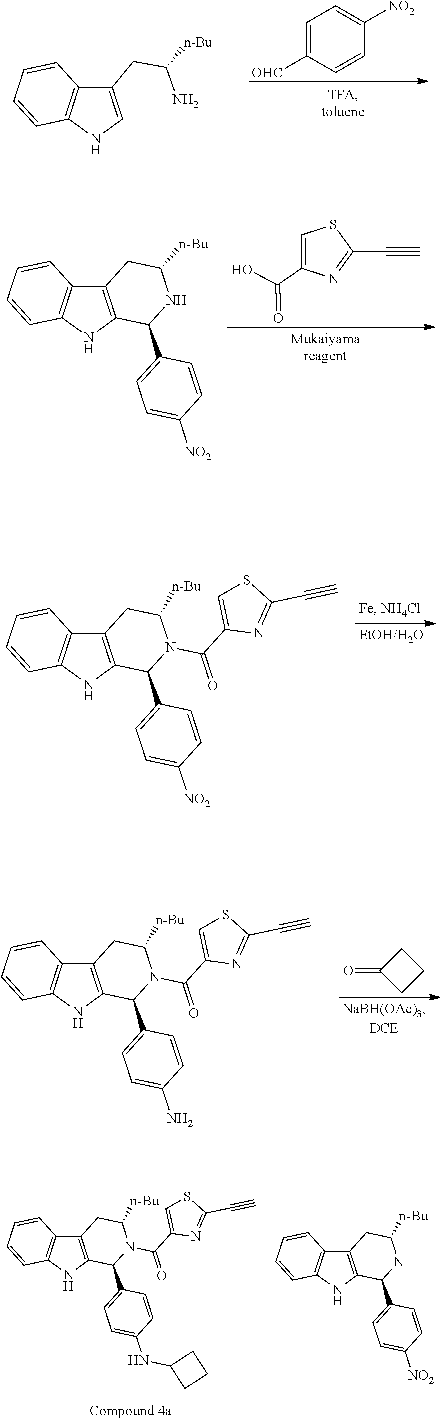

(1S,3S)-3-butyl-1-(4-nitrophenyl)-2,3,4,9-tetrahydro-1H-pyrido[3,4-b]indole: To a solution of (S)-1-(1H-indol-3-yl)hexan-2-amine (1 g, 4.62 mmol, 1 eq) in toluene (50 mL) was added 4-nitrobenzaldehyde (698.59 mg, 4.62 mmol, 154.53 uL, 1 eq) and TFA (527.09 mg, 4.62 mmol, 342.26 uL, 1 eq) at 20° C. The reaction mixture was stirred at 100° C. for 12 h to give a black green solution. The reaction mixture was monitored by TLC (PE/EA=2/1). TLC showed 4-nitrobenzaldehyde remained and some new major spots. The reaction mixture was concentrated to dryness. Then it was diluted with DCM (5 mL) and adjust pH=8 with TEA.

The residue was directly purified by flash column (SiO₂, PE to 15-30% EtOAc in PE). The combined flows were concentrated to afford (1S,3S)-3-butyl-1-(4-nitrophenyl)-2,3,4,9-tetrahydro-1H-pyrido[3,4-b]indole (340 mg, 973.03 umol, 21.05% yield) as a brown solid and (1R,3S)-3-butyl-1-(4-nitrophenyl)-2,3,4,9-tetrahydro-1H-pyrido[3,4-b]indole (1 g, 2.86 mmol, 61.91% yield) as brown oil.

((1S,3S)-3-butyl-1-(4-nitrophenyl)-1,3,4,9-tetrahydro-2H-pyrido[3,4-b]indol-2-yl)(2-ethynylthiazol-4-yl)methanone: To a solution of 2-ethynylthiazole-4-carboxylic acid (70 mg, 457.04 umol, 8.77 uL, 1 eq) in DCM (2 mL) was added 2-chloro-1-methyl-pyridin-1-ium;iodide (116.77 mg, 457.04 umol, 1 eq) at 20° C. The reaction mixture was stirred at 20° C. for 0.5 h. Then it was added into a solution of (1S,3S)-3-butyl-1-(4-nitrophenyl)-2,3,4,9-tetrahydro-1H-pyrido[3,4-b]indole: To a solution of (S)-1-(1H-indol-3-yl)hexan-2-amine (159.70 mg, 457.04 umol, 1 eq) and TEA (92.50 mg, 914.09 umol, 127.23 uL, 2 eq) in DCM (2 mL) at 0° C. The reaction mixture was stirred at 20° C. for 1 h to give a yellow solution. The reaction mixture was monitored by TLC (PE/EA=3/1). TLC showed (1S,3S)-3-butyl-1-(4-nitrophenyl)-2,3,4,9-tetrahydro-1H-pyrido[3,4-b]indole: To a solution of (S)-1-(1H-indol-3-yl)hexan-2-amine disappeared and a new major spot. The reaction mixture was poured into water (10 mL) and extracted with DCM (10 mL×3). The combined organic phases were washed with sat. brine (10 mL), dried over Na₂SO₄, filtered and concentrated to dryness. The residue was directly purified by flash column (SiO₂, PE to 20% EtOAc in PE). The combined flows were concentrated to afford ((1S,3S)-3-butyl-1-(4-nitrophenyl)-1,3,4,9-tetrahydro-2H-pyrido[3,4-b]indol-2-yl)(2-ethynylthiazol-4-yl)methanone (180 mg, 348.40 umol, 76.23% yield, 93.79% purity) as yellow oil.

((1S,3S)-1-(4-aminophenyl)-3-butyl-1,3,4,9-tetrahydro-2H-pyrido[3,4-b]indol-2-yl)(2-ethynylthiazol-4-yl)methanone: To a mixture of ((1S,3S)-3-butyl-1-(4-nitrophenyl)-1,3,4,9-tetrahydro-2H-pyrido[3,4-b]indol-2-yl)(2-ethynylthiazol-4-yl)methanone (180 mg, 371.46 umol, 1 eq) in EtOH (5 mL) and H₂O (1 mL) was added NH₄Cl (397.39 mg, 7.43 mmol, 20 eq) and Fe (207.46 mg, 3.71 mmol, 10 eq) at 25° C. The reaction mixture was stirred at 25° C. for 14 h to give a yellow suspension. The reaction mixture was monitored by TLC (PE/EA=3/1). TLC showed the reaction was completed. The reaction mixture was filtered by a pad of celite and the filtrate was concentrated to remove most of solvent. Then it was poured into water (10 mL) and extracted with EtOAc (10 mL×3). The combined organic phases were washed with sat. brine (10 mL), dried over Na₂SO₄, filtered and concentrated to dryness. No purification and used for the next step directly. Compound ((1S,3S)-1-(4-aminophenyl)-3-butyl-1,3,4,9-tetrahydro-2H-pyrido[3,4-b]indol-2-yl)(2-ethynylthiazol-4-yl)methanone (140 mg, 213.24 umol, 57.41% yield, 69.24% purity) was obtained as a yellow foam. LC-MS (m/z): 477.1 [M+Na]⁺; ¹H NMR (400 MHz, DMSO-d₆) δ=10.58 (br s, 1H), 8.02 (s, 1H), 7.45 (d, J=7.6 Hz, 1H), 7.25 (d, J=8.0 Hz, 1H), 7.08-6.94 (m, 4H), 6.48 (d, J=8.8 Hz, 2H), 6.21 (s, 1H), 4.88-4.68 (m, 2H), 4.41-4.23 (m, 1H), 3.08-2.92 (m, 2H), 1.94-1.72 (m, 1H), 1.55 (br d, J=6.0 Hz, 1H), 1.20-1.01 (m, 4H), 0.78-0.70 (m, 3H)

((1S,3S)-3-butyl-1-(4-(cyclobutylamino)phenyl)-1,3,4,9-tetrahydro-2H-pyrido[3,4-b]indol-2-yl)(2-ethynylthiazol-4-yl)methanone: To a mixture of ((1S,3S)-1-(4-aminophenyl)-3-butyl-1,3,4,9-tetrahydro-2H-pyrido[3,4-b]indol-2-yl)(2-ethynylthiazol-4-yl)methanone (40 mg, 87.99 umol, 1 eq) and cyclobutanone (7.40 mg, 105.59 umol, 7.89 uL, 1.2 eq) in DCE (2 mL) was added HOAc (5.28 mg, 87.99 umol, 5.03 uL, 1 eq) at 25° C. The mixture was stirred at 25° C. for 0.5 h. Then NaBH(OAc)₃ (37.30 mg, 175.98 umol, 2 eq) was added in the mixture. The reaction mixture was stirred at 25° C. for 14.5 h to give a yellow suspension. The reaction mixture was monitored by TLC (PE/EA=2/1). TLC showed ((1S,3S)-1-(4-aminophenyl)-3-butyl-1,3,4,9-tetrahydro-2H-pyrido[3,4-b]indol-2-yl)(2-ethynylthiazol-4-yl)methanone remained and a new major spot. The reaction mixture was poured into sat·NaHCO₃·aq (5 mL) and extracted with DCM (10 mL×3). The combined organic phases were dried over Na₂SO₄, filtered and concentrated to dryness. The residue was purified by prep-TLC (PE/EA=3/1, Rf=0.4) and lyophilized to afford ((1S,3S)-3-butyl-1-(4-(cyclobutylamino)phenyl)-1,3,4,9-tetrahydro-2H-pyrido[3,4-b]indol-2-yl)(2-ethynylthiazol-4-yl)methanone (22.47 mg, 42.29 umol, 48.06% yield, 95.73% purity) as a white powder and for delivery. LC-MS (m/z): 509.1 [M+H]⁺; ¹H NMR (400 MHz, DMSO-d₆) δ=10.56 (br s, 1H), 8.01 (s, 1H), 7.43 (d, J=7.6 Hz, 1H), 7.23 (d, J=7.6 Hz, 1H), 7.08-6.91 (m, 4H), 6.39 (d, J=8.8 Hz, 2H), 6.19 (s, 1H), 5.52 (br s, 1H), 4.84 (d, J=2.4 Hz, 1H), 4.34 (br s, 1H), 3.78 (sxt, J=7.2 Hz, 1H), 3.06-2.93 (m, 2H), 2.35-2.23 (m, 2H), 1.87-1.44 (m, 6H), 1.22-0.97 (m, 4H), 0.78-0.67 (m, 3H)

### BIOLOGICAL EXAMPLES

### Example 1: Cell Proliferation (Alamar Blue) Assay

A cell viability assay is performed to assess the potency of the compounds in human cancer cell lines 786-O (renal cell carcinoma), SJSA-1 (osteosarcoma), and/or A431 (epidermoid carcinoma). Additional cell lines, such as pancreatic cancer cell lines (e.g., Panc 02.13, BxPC-3, Panc 12, Panc 02.03, Panc 6.03, PSN-1, HPAC, and Capan-1), prostate cancer cell lines (e.g., PC-3, DU145, 22Rv1, NCI-H660, BPH1, LNCaP, BM-1604, and MDA PCa 2b), etc., can be tested in a similar method.

Cells (SJSA-1, 786-0 and/or A431) are seeded (5000 cells/100 μL/well) in 96-well tissue culture plate and incubated at 37° C./5% CO2 for 16-24 hours. The cells are then treated with compounds (25 μL of 5×). The compound concentrations are 10-0.0005 μM prepared in 3-fold serial dilutions with final DMSO concentration of 1%. The plates are then incubated for 24 h at 37° C./5% CO₂ in a moist environment. Then Alamar Blue™ reagent (final concentration 1×-12.5 μL) is added to each well and incubated for 1.5 hours at 37° C./5% CO2. The plates are read on fluorescence reader at 540 nanometer (nm) excitation and 590 nm emission wavelengths. The IC₅₀ values are subsequently determined using a sigmoidal dose-response curve (variable slope) in GraphPad Prism® 5 software.

Selected compounds are also assayed (counter-screened) in a human lung cancer cell line, A549, a less sensitive cell line to GPX4 inhibitors, as a control to assess differential activity. Cells at a density of 800-2,000 cells/well are seeded in 96-well plates and incubated at 37° C. overnight. A series of nine different concentrations of compound stocks (500×) are created by 3-fold serial dilution in DMSO. These compounds are further diluted in culture media and then added to cells so that the final DMSO concentration is equal to 0.25% or less. After 96 hours of incubation, 50 μL of CellTiter Glo reagent (Promega) is added to each well and luminescence is measured after 10 minutes using EnVision (PerkinElmer). RSL3 (a prototype GPX4 inhibitor, also known as RSL-3) is used as a reference compound titrated from top concentration of up to 30 M. All compounds are tested initially from 30 μM as the top concentration in duplicates (range of 4.6 nM-30 μM). The top concentration is then adjusted to higher (from up to 1000 M) or lower for compounds that showed potency out of the initial range. Luminescence from cells treated with DMSO alone is set as Max and % of inhibition is calculated as follows: Inhibition %=(Max-Sample value)/Max*100. Data is analyzed using XL-fit software (ID Business Solutions Ltd.). IC₅₀, relative IC₅₀, or % of top inhibition is calculated.

### Example 2: GPX4 Inhibition Assay

Studies have shown that lipophilic antioxidants, such as Ferrostatin, can rescue cells from GPX4 inhibition-induced ferroptosis. For instance, mesenchymal state GPX4-knockout cells can survive in the presence of Ferrostatin, however, when the supply of Ferrostatin is terminated, these cells undergo ferroptosis (see, e.g., Viswanathan et al., Nature 547:453-7, 2017). It has also been experimentally determined that that GPX4i can be rescued by blocking other components of the ferroptosis pathways, such as lipid ROS scavengers (Ferrostatin, Liproxstatin), lipoxygenase inhibitors, iron chelators and caspase inhibitors, which an apoptotic inhibitor does not rescue. These findings are suggestive of non-apoptotic, iron-dependent, oxidative cell death (i.e., ferroptosis). Accordingly, the ability of a molecule to induce ferroptotic cancer cell death, and that such ability is admonished by the addition of Ferrostatin, is clear indication that the molecule is an GPX4 inhibitor.

### Example 3: Method and Results of Western Blot—Gel Mobility Shift of GPX4

A mobility shift of GPX4 Western blot assay is established to assess target engagement directly in cell-based assay after incubation with compounds and in tumors from mice treated with compounds. Mobility shift can be used as a pharmacodynamic marker for GPX4 irreversible inhibitors. For cell-based assay, cells that are sensitive to GPX4 inhibitors (e.g., MiaPaCa-2) are seeded in 10 cm (2-8×10⁶ cells) and grown overnight. Cell seeding number can be adjusted proportionally based on the surface area if smaller dishes are used. Next day, cells are treated with DMSO and various compounds at indicated concentrations for a period of time (e.g., 0.5, 1, 2, 4, 6, or up to 72 hours). Cells are then lysed in 0.3-0.5 mL of RIPA buffer (Sigma) supplemented with protease inhibitors (Roche) and phosphatase inhibitors (Sigma). Lysates are assayed for protein concentration using BCA kit (Pierce). Normalized amount of lysates (20-40 jg protein/lane) are run on 4-12% or 12% NuPage gel (Life Technologies) and the proteins are transferred to the polyvinylidene fluoride (PVDF) or nitrocellulose membrane using iBlot® Transfer Stack (Life Technologies). The membranes are probed with primary antibodies shown in Table 2 at 4° C. overnight after blocking with 1×TBST containing 5% non-fat milk for one hour at room temperature. Similar antibodies from other vendors could also be used in Western blot analysis. After ishing 5 times with 1×TBS containing 0.1% Tween20, the membranes are probed with a second application of antibody (e.g., Anti-mouse-HRP, Anti-rabbit-HRP, Anti-Goat-HRP, Anti-mouse IgG Dylight 800 conjugate or Anti-rabbit IgG DyLight 680 conjugate) (1:10000; Cell signaling or similar IR to the antibodies from different vendors) at room temperature for one hour. After washing 5 times, the membranes are scanned using ImageQuant-LAS-4010 (chemiluminiscence) (GE Healthcare) if HRP-conjugated secondary antibodies are used or Odyssey@Imaging System (Licor Biosciences) if infrared conjugated secondary antibodies are used.

TABLE 2
Primary antibodies used for Western blot analysis

| Antibody Name | Vendor | Cat No. | Species | MW | | Dilution |
|---|---|---|---|---|---|---|
| β-Actin (loading | Sigma | A5441 | Mouse | 43 | kd | 1:10000 |
| control) | | | | | | |
| Vinculin (loading | Sigma | V9131 | Mouse | 116 | KD | 1:2000 |
| control) | | | | | | |
| GPX4 | Abcam | ab125066 | Rabbit | 22 | kd | 1:1000 |
| GPX4 | Abcam | ab41787 | Rabbit | 22 | kd | 1:1000 |

In DMSO treated sample, GPX4 ran as doublet—the major lower free or unbound GPX4 band and the minor upper band (likely glutathione-bound GPX4 (Cozza et al., Free Radical Biology and Medicine, Vol 112, pages 1-11, 2017)). The amount of upper band can be reduced if samples were boiled in excess amount of reducing agent dithiothreitol (DTT). GPX4 in SDS-PAGE reducing gel moved slower (appear as a larger molecular weight protein) when treated with covalent, irreversible inhibitors of GPX4 (e.g., RSL-3 and ML162) but not reversible inhibitors (e.g., ML210), presumably due to addition of the covalently linked small molecule to GPX4. Unlike glutathione-bound GPX4, the irreversible inhibitor bound GPX4 upper band cannot be reduced by excess amount of DTT. Further, distance of the GPX4 mobility shift is correlated with the molecular weight of the irreversible GPX4 inhibitor—shifted distance is bigger with larger irreversible inhibitors. Thus, this simple mobility shift of GPX4 Western blot can be used to conveniently assess direct target engagement in vitro, in cells and in tumors by irreversible inhibitors.

### Example 4: Kinact/Ki Determination for GPX4 Inhibitors

The following example shows that target engagement with GPX4 is very rapid.

Day 1—seed cells: Cells are seeded with 5×10⁵ Calu6 cells/well into 5×6-well plates.

Day 2—treat cells with Cmpd, prepare samples for gels: Cells are treated with 1, 0.75, 0.5, 0.25 and 0.1 μM inhibitor+2 μM Ferrostatin-1 for 0, 10, 20, 30, 45, 60 minutes. 10 μL of 1000×DMSO stock solutions are prepared for each compound dilution (1, 0.75, 0.5, 0.25, 0.1 mM). Complete cell culture media (EMEM+10% FBS) is prepared with 2 μM Ferrostatin-1 final conc. Drug solutions are prepared by adding 1000× inhibitors to Ferrostatin-1-supplemented media at 1× final concentration (1, 0.75, 0.5, 0.25, 0.1 μM) plus DMSO for use as a negative control.

Cell lysis buffer is prepared by diluting 5× cell lysis buffer (Cell Signaling Technology #9803) and 100× protease/phosphatase inhibitor cocktail (Cell Signaling Technology #5872) to 1× with deionized water.

Cells are treated with drug solutions in 1-hour time course. One concentration of drug added to each 6-well plate at t=60, 45, 30, 20, 10, 0 minutes. Media is aspirated from cells in 1 well of each 6-well plate and add 1 mL of media w/drug+Ferrostatin (t=60 min). Cells are returned to incubator between time points. Media is aspirated and drug added to cells at each subsequent time point. At t=10 min DMSO is added negative control to additional well.

At t=0 media is aspirated from cells, cells are ished with ice cold PBS and aspirated, 75 μL of 1× cell lysis buffer is added per well, bottom of plates scraped with cell scraper, and lysates transferred to 1.5 mL Eppendorf tubes at store at −20° C.

SDS-PAGE running buffer is prepared (2 L of 1×MES Bolt running buffer (ThermoFisher Scientific #B0002), Store at 4° C. overnight for use the next day).

Day 3—perform BCA assay and run gels: Lysates are thawed on ice, centrifuged at 18,000×g at 4° C. for 10 minutes, and BCA assay is performed on supernatant following manufacturer protocol (ThermoFisher Scientific #23225). 3.6×LDS/BME sample buffer is prepared by mixing Bolt 4×LDS sample buffer (ThermoFisher Scientific #B0008) with 2-mercaptoethanol at a 10:1 ratio. In 96-well PCR plate 19 μL 3.6×LDS/BME sample buffer is added and 50 μL lysate samples. Lysates diluted to 1 mg/mL with 1×LDS/BME, plates heated at 95° C. for 10 min in PCR machine, loaded 15 μL/well (15 μg total lysate) into 12% Bis-Tris Bolt gels, and gels are run at 200V for −35 minutes (until dye front reaches bottom of gel) with cold 1×MES running buffer. After which time, gels are ished 5 minutes in water, 10 minutes in 20% Ethanol/water, and transferred to membrane with iBlot2 (ThermoFisher Scientific). Membrane is blocked 1 h at RT with Licor TBS blocking buffer (Licor #927-60001) and incubated with 1:1000 dilution of anti-GPX4 antibody (Abcam #ab125066) in Licor TBS blocking buffer at 4° C. overnight with gentle rocking.

Day 4—develop blots, quantify gel shift: Membrane is ished with 1×TBST for 30 minutes (change ish buffer 3-4 times), incubated with Licor secondary antibody (Licor #926-68021) 1:40,000 in Licor TBS blocking buffer for 1 h at RT with gentle rocking, ished with 1×TBST for 30 minutes, scraped with Licor imager and bands are quantized with Image studio.

### Example 5: Pharmacokinetics Studies

Male Balb/c mice (˜6-8 weeks old with body weight range of 22-25 g) and male SD rats (6-8 weeks old with body weight range of 200-250 g) are procured from Vivo Biotech, Hyderabad, India. Animals are quarantined in Jubilant Biosys Animal House for a period of 7 days with a 12:12 h light: dark cycles, and prior to the study the animals are stratified as per body weight.

Housing: The animals are group housed in standard polycarbonate cages, with stainless steel top grill where pelleted food and drinking water bottle are placed; corn cob is used as bedding material and changed at least twice a week or as required.

Diet ad libitum: Rodent feed manufactured by Altromin Spezialfutter GmbH & Co. KG., ImSeelenkamp20. D-32791 Lage, is provided.

Water ad libitum: Purified water is provided ad libitum to animals in polycarbonate bottles with stainless steel sipper tubes.

A) Procedure for Mice: Intravenous, oral, and intraperitoneal pharmacokinetics study is done at doses of 5, 20 and 10 mg/kg respectively at dose volume of 10 mL/Kg for PO and IP while 5 mL/kg for IV route. Sparse sampling is done and at each time point three mice are used for blood sampling (˜100 μL) are collected from retro-orbital plexus at 0.083 (Only for IV), 0.25, 0.5, 1, 2, 4, 8, 10 (only for PO) and 24 h. Blood samples collected in tubes containing K₂·EDTA as anticoagulant and centrifuged for 5 min at 10,000 rpm in a refrigerated centrifuge (Biofuge, Heraeus, Germany) maintained at 4° C. for plasma separation.

Group I (IV) received test compound intravenously by tail vein at 5 mg/Kg in solution formulation prepared using 30% Kolliphore EL in WFI; dose volume: 5 mL/Kg; strength: 1 mg/mL.

Group II (PO) received test compound by per oral route using oral gavage needle at 20 mg/Kg in solution formulation prepared using 30% Kolliphore EL in WFI; dose volume: 10 mL/Kg; strength: 2 mg/mL.

Group III (IP) received test compound by intraperitoneal route at 10 mg/Kg in solution formulation prepared using 30% Kolliphore EL in WFI; dose volume: 10 mL/Kg; strength: 1 mg/mL.

B) Procedure for rat: Intravenous and oral pharmacokinetics study is done at a dose 2 and 10 mg/kg at dose volume of 2 and 10 mL/Kg. Serial blood sampling is done and at each time point (˜200 μL) are collected from retro-orbital plexus at 0.083 (only for IV), 0.25, 0.5, 1, 2, 4, 8, 10 (only for PO) and 24 h. Blood samples collected in tubes containing K₂·EDTA as anticoagulant and centrifuged for 5 min at 10,000 rpm in a refrigerated centrifuge (Biofuge, Heraeus, Germany) maintained at 4° C. for plasma separation.

Group I (IV) received test compound intravenously by tail vein at 2 mg/Kg in solution formulation prepared using 30% Kolliphore EL in WFI; dose volume: 2 mL/Kg; strength: 1 mg/mL.

Group II (PO) received test compound using oral gavage needle at 10 mg/Kg (solution formulation prepared using 30% Kolliphore EL in WFI; dose volume: 10 mL/Kg: strength: 1 mg/mL.

### Example 6: Potency and Stability of Selected Compounds

Selected compounds were evaluated according to, e.g., procedures set forth in Examples 1-5.

Table 3 summarizes the 24-hour 2D viability for Compound 1a in cell lines 786-0 and A431. Table 4 summarizes the whole blood stability for Compound 1a in several species.

TABLE 3
Viability of selected compounds.

| | EC50 (nM) | | |
|---|---|---|---|
| | 786-0 | | A431 |
| Compound | 10% FBS | 10% FBS + 2 μM Fer-1 | 10% FBS |
| 1a | 43.7 | >10000 | >10000 |
| 1a | 43.7 | >10000 | >10000 |
| 2a | 29.9 | 5000 | >10000 |
| 3a | 70.2 | 3690 | >10000 |
| 4a | 43.3 | >10000 | >10000 |
| 5a | 48.9 | 3680 | 3980 |
| 6a | 36.9 | >10000 | >10000 |
| 7a | 16 | >10000 | 8410 |
| 8a | 386 | >10000 | >10000 |
| 9a | 60 | >10000 | 5160 |
| 10aA | 118 | 7530 | >10000 |
| 10aB | 380 | 5890 | >10000 |
| 11a | 78.6 | >10000 | >10000 |
| 12a | 7.6 | 8950 | NT |
| 13a | 68.3 | 4830 | 5120 |
| 14a | 72.2 | >10000 | >10000 |
| 15a | 73.1 | 4140 | >10000 |
| 16a | 85.4 | >10000 | >10000 |
| 17a | 43 | NT | NT |
| 18a | 57.1 | 8284 | NT |
| 19a | 40 | NT | NT |
| 20a | 117 | >10000 | >10000 |
| 21a | 91.5 | >10000 | >10000 |
| 22a | 20.6 | 8490 | 3270 |
| 23a | 21.1 | >10000 | 3720 |
| 24a | >10000 | >10000 | >10000 |
| 26a | 41.3 | 8760 | >10000 |
| 27a | >10000 | >10000 | 7790 |
| 28a | 44.3 | 2100 | >10000 |

TABLE 4
Whole blood stability of selected compounds.

| | T_{1/2} (min) | | |
|---|---|---|---|
| Compound | Mouse | Rat | Dog |
| 1a | 210.8 | >289.1 | 249.3 |

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

What is claimed is:

1. A compound of Formula I: 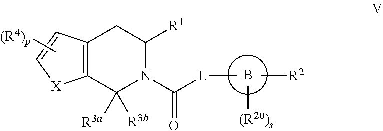
or a tautomer, stereoisomer, mixture of stereoisomers, isotopically enriched analog, or pharmaceutically acceptable salt thereof, wherein:
ring B is a heteroaryl;
L is absent or —NR²¹—;
X is —O—, —S—, —NR⁹—, —CR⁵═CR⁵—, or —CR⁵═N—;
p is 0, 1, or 2;
s is 0, 1, 2, or 3;
R¹ is C₁-C₆alkyl, C₂-C₆alkenyl, C₂-C₆alkynyl, C₁-C₆haloalkyl, C₃-C₁₀cycloalkyl, heteroaryl, —CN, —OR⁷, —C(O)OR⁶, —C(O)N(R⁷)₂, —OC(O)R⁶, —S(O)₂R⁸, —S(O)₂N(R⁷)₂, —S(O)N(R⁷)₂, —S(O)R⁸, —N(R⁷)₂, —NO₂, —C₁-C₆alkyl-OR⁷, or —Si(R¹⁵)₃; wherein the heteroaryl is unsubstituted or substituted with C₁-C₆alkyl;
R² is C₂alkynyl that is unsubstituted or substituted with C_{G}-C₆alkyl, C₂-C₆alkenyl, C₂-C₆alkynyl, C₃-C₁₀cycloalkyl, C₆-C₁₄aryl, C₇-C₁₅aralkyl, heteroaryl, or heterocyclyl;
R^{3a} is hydrogen and R^{3b} is 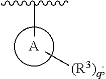
wherein q is 0, 1, 2, or 3; and ring A is C₄-C₁₀cycloalkyl, heterocyclyl, aryl, or heteroaryl; or
R^{3a} and R^{3b} together with the carbon atom to which they are attached form a C₄-C₁₀cycloalkyl or heterocyclyl; wherein the C₄-C₁₀cycloalkyl or heterocyclyl is unsubstituted or substituted with one to three R³;
each R³ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)N(R⁷)₂, —OC(O)R⁸, —C(O)R⁶, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R³ is independently unsubstituted or substituted with one to three R¹⁰;
each R⁴ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R⁴ is independently unsubstituted or substituted with one to three R¹⁰;
each R⁵ is independently hydrogen, halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, or —C₂-C₆alkenylheteroaryl of R⁵ is independently unsubstituted or substituted with one to three R¹⁰;
each R⁶ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, or —C₂-C₆alkenylheteroaryl; wherein each R⁶ is independently unsubstituted or substituted with one to three R¹¹;
each R⁷ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₆cycloalkyl, —C₂-C₆alkenylC₃-C₆cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, —C₂-C₆alkenylheteroaryl, or two R⁷ together with the nitrogen atom to which they are attached, form a 4 to 7 membered heterocyclyl; wherein each R⁷ or ring formed thereby is independently unsubstituted or substituted with one to three R¹¹;
each R⁸ is independently C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, or —C₂-C₆alkenylheteroaryl; wherein each R⁸ is independently unsubstituted or substituted with one to three R¹¹;
R⁹ is hydrogen or C₁-C₆alkyl;
each R¹⁰ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, —P(O)(OR¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl, wherein each C₁-C₆alkyl, C₁-C₆haloalkyl, C₂-C₆alkenyl, C₂-C₆alkynyl, C₃-C₁₀cycloalkyl, heterocyclyl, aryl, or heteroaryl of R¹⁰ is independently unsubstituted or substituted with one to three R¹¹;
each R¹¹ is independently halo, —CN, —OR¹², —NO₂, —N(R¹²)₂, —S(O)R³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl;
each R¹² is independently hydrogen, C₁-C₆alkyl or C₃-C₁₀cycloalkyl;
each R¹³ is independently C₁-C₆alkyl or C₃-C₁₀cycloalkyl;
each R¹⁵ is independently C₁-C₆alkyl, C₂-C₆alkenyl, aryl, heteroaryl, —C₁-C₆alkylaryl, —C₂-C₆alkenylaryl, —C₁-C₆alkylheteroaryl, or —C₂-C₆alkenylheteroaryl;
each R²⁰ is independently halogen, C₁-C₆alkyl, or C₂-C₆alkenyl; and
R²¹ is hydrogen or C₁-C₆alkyl.

2. The compound of claim 1, wherein the compound is a compound according to Formula IA or IB: 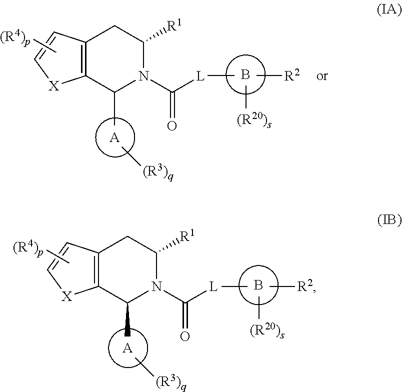
or a tautomer, stereoisomer, mixture of stereoisomers, isotopically enriched analog, or pharmaceutically acceptable salt thereof.

3. The compound of claim 1 or 2, wherein ring B includes at least one heteroatom selected from S, O, and N.

4. The compound of any one of claims 1-3, wherein ring B has the structure: 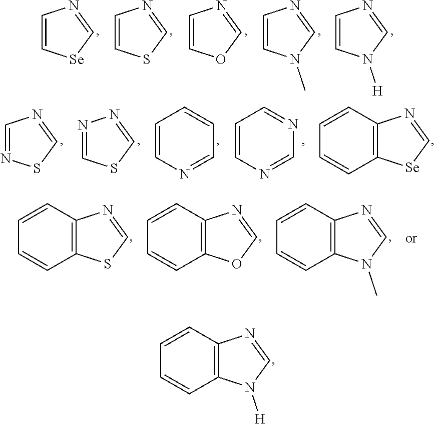
which structure is substituted with R² and s R²⁰s.

5. The compound of claim 4, wherein ring B has the structure 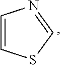
which structure is substituted with R² and s R²⁰s.

6. The compound of any one of claims 1-5, wherein the compound is a compound according to Formula IC: 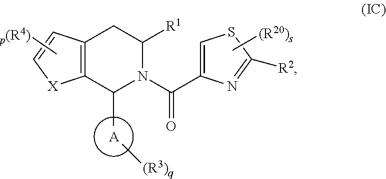
or a tautomer, stereoisomer, mixture of stereoisomers, isotopically enriched analog, or pharmaceutically acceptable salt thereof.

7. The compound of claim 6, wherein the compound is a compound according to Formula ID: 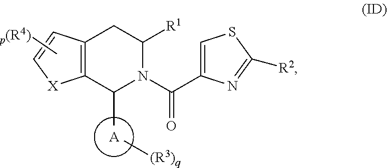
or a tautomer, stereoisomer, mixture of stereoisomers, isotopically enriched analog, or pharmaceutically acceptable salt thereof.

8. The compound of claim 7, wherein the compound is a compound according to Formula IE: 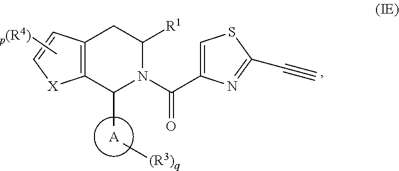
or a tautomer, stereoisomer, mixture of stereoisomers, isotopically enriched analog, or pharmaceutically acceptable salt thereof.

9. The compound of any one of claims 1-5, wherein the compound is a compound according to Formula IF: 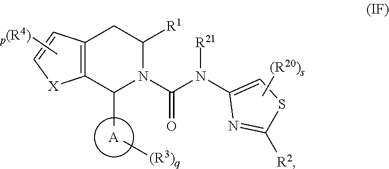
or a tautomer, stereoisomer, mixture of stereoisomers, isotopically enriched analog, or pharmaceutically acceptable salt thereof.

10. The compound of claim 9, wherein the compound is a compound according to Formula IG: 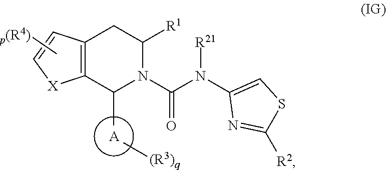
or a tautomer, stereoisomer, mixture of stereoisomers, isotopically enriched analog, or pharmaceutically acceptable salt thereof.

11. The compound of claim 10, wherein the compound is a compound according to Formula IH: 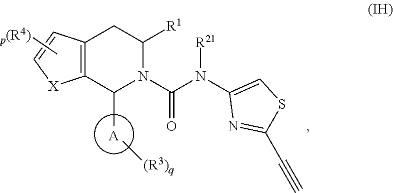
or a tautomer, stereoisomer, mixture of stereoisomers, isotopically enriched analog, or pharmaceutically acceptable salt thereof.

12. The compound of any one of claims 1-11, wherein X is —CH═CH—.

13. The compound of any one of claims 1-5, wherein the compound is a compound according to Formula IJ: 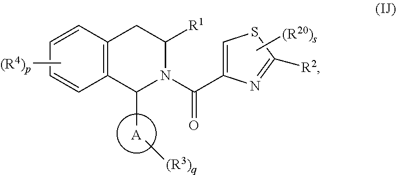
or a tautomer, stereoisomer, mixture of stereoisomers, isotopically enriched analog, or pharmaceutically acceptable salt thereof.

14. The compound of claim 13, wherein the compound is a compound according to Formula IK: 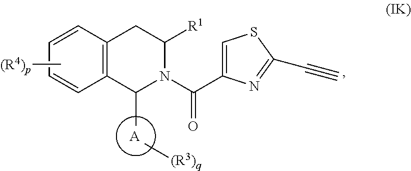
or a tautomer, stereoisomer, mixture of stereoisomers, isotopically enriched analog, or pharmaceutically acceptable salt thereof.

15. The compound of any one of claims 1-5, wherein the compound is a compound according to Formula IL: 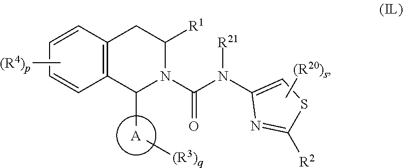
or a tautomer, stereoisomer, mixture of stereoisomers, isotopically enriched analog, or pharmaceutically acceptable salt thereof.

16. The compound of claim 15, wherein the compound is a compound according to Formula IM: 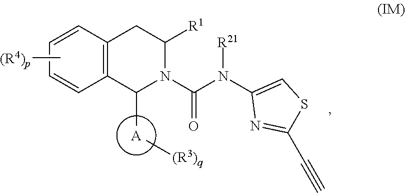
or a tautomer, stereoisomer, mixture of stereoisomers, isotopically enriched analog, or pharmaceutically acceptable salt thereof.

17. The compound of any one of claims 9-11, 15, or 16, wherein R²¹ is H.

18. The compound of any one of claims 1-17, wherein ring A is C₄-C₁₀cycloalkyl or heterocyclyl.

19. The compound of any one of claims 1-17, wherein ring A is aryl or heteroaryl.

20. The compound of claim 19, wherein ring A is phenyl or quinoline.

21. The compound of claim 20, wherein ring A is phenyl.

22. The compound of claim 21, wherein the compound is a compound according to Formula IN: 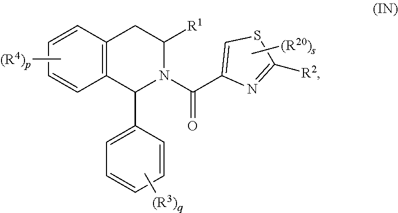
or a tautomer, stereoisomer, mixture of stereoisomers, isotopically enriched analog, or pharmaceutically acceptable salt thereof.

23. The compound of claim 22, wherein the compound is a compound according to Formula IP: 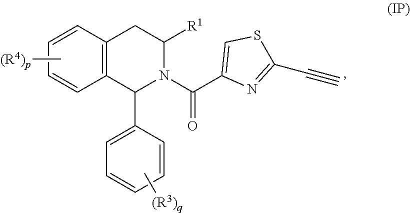
or a tautomer, stereoisomer, mixture of stereoisomers, isotopically enriched analog, or pharmaceutically acceptable salt thereof.

24. The compound of claim 21, wherein the compound is a compound according to Formula IQ: 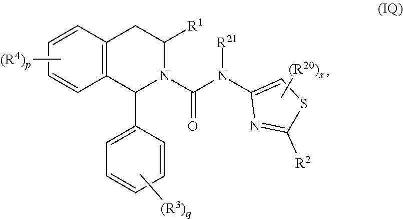
or a tautomer, stereoisomer, mixture of stereoisomers, isotopically enriched analog, or pharmaceutically acceptable salt thereof.

25. The compound of claim 24, wherein the compound is a compound according to Formula IR: 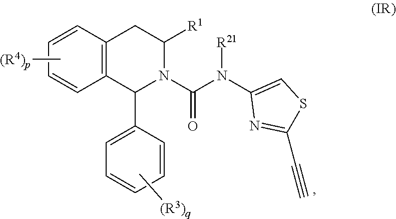
or a tautomer, stereoisomer, mixture of stereoisomers, isotopically enriched analog, or pharmaceutically acceptable salt thereof.

26. The compound of any one of claims 1-25, wherein q is 0.

27. The compound of any one of claims 1-25, wherein q is 1, 2, or 3.

28. The compound of claim 27, wherein q is 1.

29. The compound of claim 27, wherein q is 2 or 3.

30. The compound of any one of claims 27-29, wherein at least one R³ is halo, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)R⁸, —C(O)R⁶, or —OC(O)CHR⁸N(R¹²)₂.

31. The compound of claim 30, wherein at least one R³ is halo.

32. The compound of claim 30 or 31, wherein at least one R³ is —NHR⁸.

33. The compound of claim 32, wherein at least one R³ is —NH(adamantyl) or —NH(adamantyl-(C₁-C₆aliphatic)).

34. The compound of any one of claims 27-33, wherein each R³ is independently —NH₂, fluoro, methyl, pyridine-4-carboxamido, pyridin-3-amino, pentyloxycarbonylamino, N-(3-aminobicyclo[1.1.1]pentan-1-yl)amino, morpholin-4-yl, methoxycarbonyl, dimethylcarbamoyl, cyclopropylcarbamoyl, cyclohexyl, cyclobutylcarbamoyl, cyclobutylaminosulfonyl, adamantylamino, (adamantan-1-ylamino)methyl, 3-methyl-1,2,4-oxadiazol-5-yl, 2-methylpyridine-4-carboxamido, (bicyclo[1.1.1]pentan-1-ylamino)methyl, (adamantan-1-yl)carbamoyl, or (2-methoxyethyl)carbamoyl.

35. The compound of any one of claims 1-34, wherein R¹ is C₁-C₆alkyl, C₂-C₆alkenyl, C₂-C₆alkynyl, C₁-C₆haloalkyl, C₃-C₁₀cycloalkyl, —CN, —C(O)OR⁶, —C(O)N(R⁷)₂, —N(R⁷)₂, —OR⁷, or —C₁-C₆alkyl-OR⁷.

36. The compound of claim 35, wherein R¹ is C₁-C₆alkyl.

37. The compound of claim 36, wherein R¹ is n-butyl.

38. The compound of any one of claims 1-37, wherein p is 0.

39. The compound of any one of claims 1-37, wherein p is 1, 2, or 3.

40. The compound of claim 39, wherein p is 1.

41. The compound of claim 39, wherein p is 2 or 3.

42. The compound of any one of claims 39-41, wherein each R⁴ is independently halo, —CN, —OH, —OR⁸, —NH₂, —NHR⁸, —N(R⁸)₂, —S(O)₂R⁸, —S(O)R⁵, —S(O)₂N(R′)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, C₁-C₆alkyl, C₂-C₆alkenyl, C₂-C₆alkynyl, or C₃-C₁₀cycloalkyl; wherein each C₁-C₆alkyl, C₂-C₆alkenyl, C₂-C₆alkynyl, or C₃-C₁₀cycloalkyl of R⁴ is independently optionally substituted with one to three R¹⁰.

43. The compound of claim 42, wherein each R⁴ is independently halo, —CN, —OR⁷, C₁-C₆alkyl, C₂-C₆alkynyl, or C₃-C₁₀cycloalkyl; wherein each C₁-C₆alkyl, C₂-C₆alkynyl, or C₃-C₁₀cycloalkyl of R⁴ is independently optionally substituted with one to three R¹⁰.

44. The compound of claim 43, wherein each R⁴ is independently halo, —CN, —OH, —OR⁸, C₁-C₆alkyl, or C₂-C₆alkynyl; wherein the C₁-C₆alkyl of R⁴ is unsubstituted or substituted with one to three R¹⁰.

45. The compound of any one of claims 1-44, wherein s is 0.

46. The compound of any one of claims 1-44, wherein s is 1, 2, or 3.

47. A compound of Formula II: 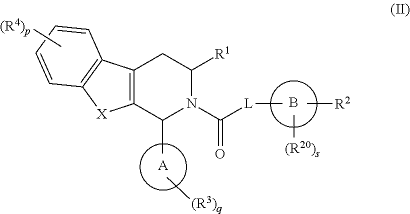
or a tautomer, stereoisomer, mixture of stereoisomers, isotopically enriched analog, or pharmaceutically acceptable salt thereof, wherein:
ring A is C₄-C₁₀cycloalkyl, heterocyclyl, aryl, or heteroaryl;
ring B is a heteroaryl;
X is —NR⁵—, —O—, or —S—;
L is absent or —NR⁹—;
p is 0, 1, 2, or 3;
q is 0, 1, 2, or 3;
s is 0, 1, 2, or 3;
R¹ is C₁-C₆alkyl, C₂-C₆alkenyl, C₂-C₆alkynyl, C₁-C₆haloalkyl, C₃-C₁₀cycloalkyl, —CN, —OH, —C(O)OR⁶, —C(O)N(R⁷)₂, —OC(O)R⁶, —S(O)₂R⁸, —S(O)₂N(R⁷)₂, —S(O)N(R⁷)₂, —S(O)R⁸, —NH₂, —NHR⁸, —N(R⁸)₂, —NO₂, —OR⁸, —C₁-C₆alkyl-OH, —C₁-C₆alkyl-OR⁸, or —Si(R¹⁵)₃;
R² is C₂alkynyl that is unsubstituted or substituted with C₁-C₆alkyl, C₂-C₆alkenyl, C₂-C₆alkynyl, C₃-C₁₀cycloalkyl, C₆-C₁₄aryl, C₇-C₁₅aralkyl, heteroaryl, or heterocyclyl;
each R³ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)N(R⁷)₂, —OC(O)R⁸, —C(O)R⁶, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R³ is independently unsubstituted or substituted with one to three R¹⁰;
each R⁴ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, —NR¹²C(O)OR⁸, —OC(O)N(R⁷)₂, —OC(O)CHR⁸N(R¹²)₂, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl of R⁴ is independently unsubstituted or substituted with one to three R¹⁰;
R⁵ is hydrogen or C₁-C₆alkyl;
each R⁶ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, C₁-C₆alkylheteroaryl, or —C₂-C₆alkenylheteroaryl; wherein each R⁶ is independently unsubstituted or substituted with one to three R¹¹;
each R⁷ is independently hydrogen, C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₆cycloalkyl, —C₂-C₆alkenylC₃-C₆cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, —C₂-C₆alkenylheteroaryl, or two R⁷ together with the nitrogen atom to which they are attached, form a 4 to 7 membered heterocyclyl; wherein each R⁷ or ring formed thereby is independently unsubstituted or substituted with one to three R¹¹;
each R⁸ is independently C₁-C₆alkyl, C₂-C₆alkenyl, C₂-C₆alkynyl, C₃-C₁₀cycloalkyl, heterocyclyl, aryl, heteroaryl, —C₁-C₆alkylC₃-C₁₀cycloalkyl, —C₂-C₆alkenylC₃-C₁₀cycloalkyl, —C₁-C₆alkylheterocyclyl, —C₂-C₆alkenylheterocyclyl, —C₁-C₆alkylaryl, —C₂-C₆alkenylaryl, —C₁-C₆alkylheteroaryl, or —C₂-C₆alkenylheteroaryl; wherein each R⁸ is independently unsubstituted or substituted with one to three R¹¹;
R⁹ is hydrogen or C₁-C₆alkyl;
each R¹⁰ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, —P(O)(OR¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl, wherein each C₁-C₆alkyl, C₁-C₆haloalkyl, C₂-C₆alkenyl, C₂-C₆alkynyl, C₃-C₁₀cycloalkyl, heterocyclyl, aryl, or heteroaryl of R¹⁰ is independently unsubstituted or substituted with one to three R¹¹;
each R¹¹ is independently halo, —CN, —OR¹², —NO₂, —N(R¹²)₂, —S(O)R¹³, —S(O)₂R¹³, —S(O)N(R¹²)₂, —S(O)₂N(R¹²)₂, —Si(R¹²)₃, —C(O)R¹², —C(O)OR¹², —C(O)N(R¹²)₂, —NR¹²C(O)R¹², —OC(O)R¹², —OC(O)OR¹², —OC(O)N(R¹²)₂, —NR¹²C(O)OR¹², —OC(O)CHR¹²N(R¹²)₂, C₁-C₆alkyl, C₁-C₆haloalkyl, C₂-C₆alkenyl, C₂-C₆alkynyl, C₃-C₁₀cycloalkyl, heterocyclyl, aryl, or heteroaryl;
each R¹² is independently hydrogen, C₁-C₆alkyl, or C₃-C₁₀cycloalkyl;
each R¹³ is independently C₁-C₆alkyl, or C₃-C₁₀cycloalkyl;
each R¹⁵ is independently C—C₆alkyl, C₂-C₆alkenyl, aryl, heteroaryl, —C₁-C₆alkylaryl, —C₂-C₆alkenylaryl, —C₁-C₆alkylheteroaryl, and —C₂-C₆alkenylheteroaryl; and
each R²⁰ is independently halogen, C₁-C₆alkyl, or C₂-C₆alkenyl.

48. The compound of claim 47, wherein ring B includes at least one heteroatom selected from S, O, and N.

49. The compound of claim 47 or 48, wherein ring B has the structure: 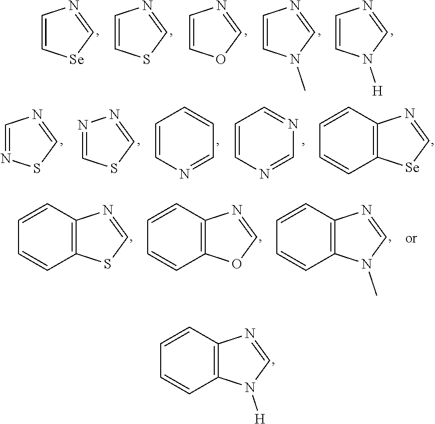
which structure is substituted with R² and s R²⁰s.

50. The compound of claim 49, wherein ring B has the structure 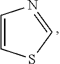
which structure is substituted with R² and s R²⁰s.

51. The compound of any one of claims 47-50, wherein the compound is a compound according to Formula IIA: 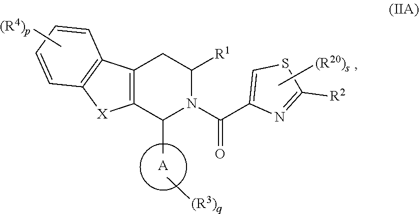
or a tautomer, stereoisomer, mixture of stereoisomers, isotopically enriched analog, or pharmaceutically acceptable salt thereof.

52. The compound of claim 51, wherein the compound is a compound according to Formula IIA: 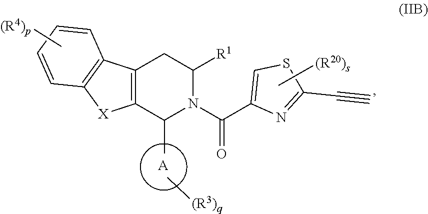
or a tautomer, stereoisomer, mixture of stereoisomers, isotopically enriched analog, or pharmaceutically acceptable salt thereof.

53. The compound of any one of claims 47-50, wherein the compound is a compound according to Formula IIC: 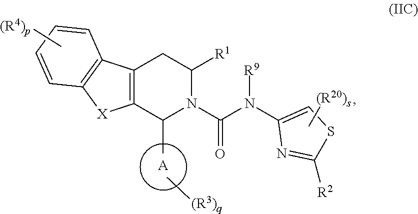
or a tautomer, stereoisomer, mixture of stereoisomers, isotopically enriched analog, or pharmaceutically acceptable salt thereof.

54. The compound of claim 53, wherein the compound is a compound according to Formula IID: 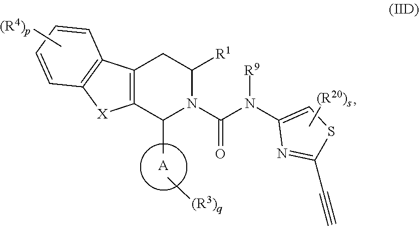
or a tautomer, stereoisomer, mixture of stereoisomers, isotopically enriched analog, or pharmaceutically acceptable salt thereof.

55. The compound of any one of claims 47-50, wherein the compound is a compound according to Formula IIE: 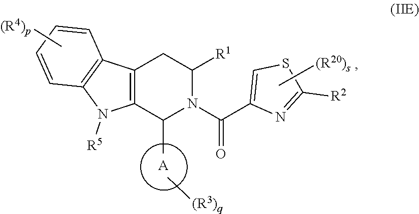
or a tautomer, stereoisomer, mixture of stereoisomers, isotopically enriched analog, or pharmaceutically acceptable salt thereof.

56. The compound of claim 55, wherein the compound is a compound according to Formula IIF: 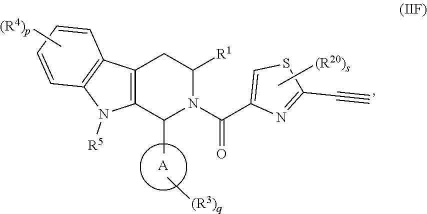
or a tautomer, stereoisomer, mixture of stereoisomers, isotopically enriched analog, or pharmaceutically acceptable salt thereof.

57. The compound of any one of claims 47-50, wherein the compound is a compound according to Formula IIG: 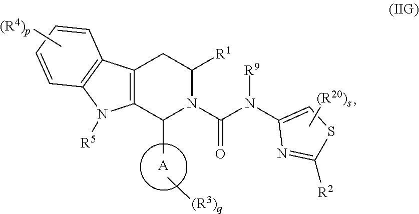
or a tautomer, stereoisomer, mixture of stereoisomers, isotopically enriched analog, or pharmaceutically acceptable salt thereof.

58. The compound of claim 57, wherein the compound is a compound according to Formula IIF: 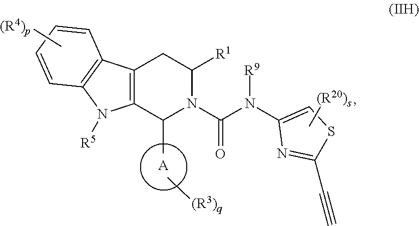
or a tautomer, stereoisomer, mixture of stereoisomers, isotopically enriched analog, or pharmaceutically acceptable salt thereof.

59. The compound of any one of claims 47-58, wherein ring A is C₄-C₁₀cycloalkyl or heterocyclyl.

60. The compound of any one of claims 47-58, wherein ring A is aryl or heteroaryl.

61. The compound of claim 60, wherein ring A is phenyl or quinoline.

62. The compound of claim 61, wherein ring A is phenyl.

63. The compound of claim 62, wherein the compound is a compound according to Formula IIJ: 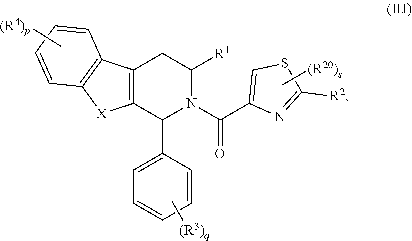
or a tautomer, stereoisomer, mixture of stereoisomers, isotopically enriched analog, or pharmaceutically acceptable salt thereof.

64. The compound of claim 63, wherein the compound is a compound according to Formula IIK: 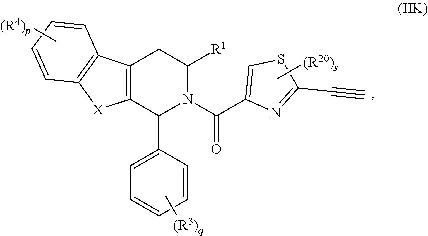
or a tautomer, stereoisomer, mixture of stereoisomers, isotopically enriched analog, or pharmaceutically acceptable salt thereof.

65. The compound of claim 64, wherein the compound is a compound according to Formula IIL: 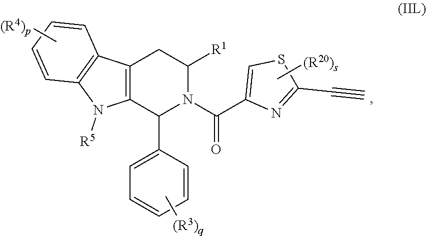
or a tautomer, stereoisomer, mixture of stereoisomers, isotopically enriched analog, or pharmaceutically acceptable salt thereof.

66. The compound of claim 62, wherein the compound is a compound according to Formula IIM: 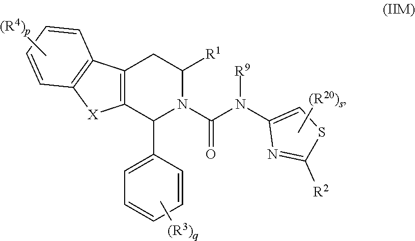
or a tautomer, stereoisomer, mixture of stereoisomers, isotopically enriched analog, or pharmaceutically acceptable salt thereof.

67. The compound of claim 66, wherein the compound is a compound according to Formula IIN: 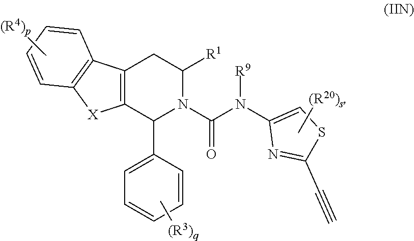
or a tautomer, stereoisomer, mixture of stereoisomers, isotopically enriched analog, or pharmaceutically acceptable salt thereof.

68. The compound of claim 67, wherein the compound is a compound according to Formula IP: 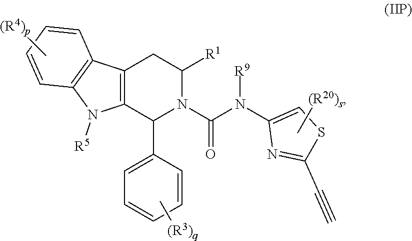
or a tautomer, stereoisomer, mixture of stereoisomers, isotopically enriched analog, or pharmaceutically acceptable salt thereof.

69. The compound of any one of claims 47-68, wherein R⁹ is H.

70. The compound of any one of claims 47-69, wherein R⁵ is H.

71. The compound of any one of claims 47-70, wherein q is 0.

72. The compound of any one of claims 47-70, wherein q is 1, 2, or 3.

73. The compound of claim 72, wherein q is 1.

74. The compound of claim 72, wherein q is 2 or 3.

75. The compound of any one of claims 72-74, wherein at least one R³ is halo, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹²)₃, —SF₅, —C(O)OR⁶, —C(O)N(R⁷)₂, —NR¹²C(O)R⁸, —NR¹²C(O)OR⁸, —OC(O)R⁸, —C(O)R⁶, or —OC(O)CHR⁸N(R¹²)₂.

76. The compound of claim 75, wherein at least one R³ is halo.

77. The compound of claim 75 or 76, wherein at least one R³ is —NHR⁸.

78. The compound of claim 77, wherein at least one R³ is —NH(adamantyl) or —NH(adamantyl-(C₁-C₆aliphatic)).

79. The compound of any one of claims 72-78, wherein each R³ is independently —NH₂, fluoro, methyl, pyridine-4-carboxamido, pyridin-3-amino, pentyloxycarbonylamino, N-(3-aminobicyclo[1.1.1]pentan-1-yl)amino, morpholin-4-yl, methoxycarbonyl, dimethylcarbamoyl, cyclopropylcarbamoyl, cyclohexyl, cyclobutylcarbamoyl, cyclobutylaminosulfonyl, adamantylamino, (adamantan-1-ylamino)methyl, 3-methyl-1,2,4-oxadiazol-5-yl, 2-methylpyridine-4-carboxamido, (bicyclo[1.1.1]pentan-1-ylamino)methyl, (adamantan-1-yl)carbamoyl, or (2-methoxyethyl)carbamoyl.

80. The compound of any one of claims 47-79, wherein R¹ is C₁-C₆alkyl, C₂-C₆alkenyl, C₂-C₆alkynyl, C₁-C₆haloalkyl, C₃-C₁₀cycloalkyl, —CN, —C(O)OR⁶, —C(O)N(R⁷)₂, —NH₂, —NHR⁸, —N(R⁸)₂, —OH, —OR⁸, —C₁-C₆alkyl-OH, or —C₁-C₆alkyl-OR⁸.

81. The compound of claim 80, wherein R¹ is C₁-C₆alkyl.

82. The compound of claim 81, wherein R¹ is n-butyl.

83. The compound of any one of claims 47-82, wherein p is 0.

84. The compound of any one of claims 47-82, wherein p is 1, 2, or 3.

85. The compound of claim 84, wherein p is 1.

86. The compound of claim 85, wherein p is 2 or 3.

87. The compound of any one of claims 84-86, wherein each R⁴ is independently halo, —CN, —OH, —OR⁸, —NH₂, —NHR⁸, —N(R⁸)₂, —S(O)₂R⁸, —S(O)R⁸, —S(O)₂N(R⁷)₂, —S(O)N(R⁷)₂, —NO₂, —Si(R¹⁵)₃, —C(O)OR⁶, —C(O)N(R⁷)₂, —NR¹²C(O)R⁸, —OC(O)R⁸, —C(O)R⁶, C₁-C₆alkyl, C₂-C₆alkenyl, C₂-C₆alkynyl, or C₃-C₁₀cycloalkyl; wherein each C₁-C₆alkyl, C₂-C₆alkenyl, C₂-C₆alkynyl, or C₃-C₁₀cycloalkyl of R⁴ is independently optionally substituted with one to three R¹⁰.

88. The compound of claim 87, wherein each R⁴ is independently halo, —CN, —OR⁷, C₁-C₆alkyl, C₂-C₆alkynyl, or C₃-C₁₀cycloalkyl; wherein each C₁-C₆alkyl, C₂-C₆alkynyl, or C₃-C₁₀cycloalkyl of R⁴ is independently optionally substituted with one to three R¹⁰.

89. The compound of claim 88, wherein each R⁴ is independently halo, —CN, —OH, —OR⁸, C₁-C₆alkyl, or C₂-C₆alkynyl; wherein the C₁-C₆alkyl of R⁴ is unsubstituted or substituted with one to three R¹⁰.

90. The compound of any one of claims 47-89, wherein s is 0.

91. The compound of any one of claims 47-89, wherein s is 1, 2, or 3.

92. A compound selected from the group consisting of the compounds listed in Table 1, or a tautomer, stereoisomer, mixture of stereoisomers, isotopically enriched analog, or pharmaceutically acceptable salt thereof.

93. A pharmaceutical composition comprising a compound, or a tautomer, stereoisomer, mixture of stereoisomers, isotopically enriched analog, or pharmaceutically acceptable salt thereof, of any one of claims 1-92, and a pharmaceutically acceptable carrier.

94. A method of inhibiting GPX4 in a cell, comprising contacting a cell with an effective amount of a compound, or a tautomer, stereoisomer, mixture of stereoisomers, isotopically enriched analog, or pharmaceutically acceptable salt thereof, of any one of claims 1-92.

95. The method of claim 94, wherein the cell is located within a subject.

96. The method of claim 95, wherein the subject is a human.

97. The method of any one of claims 94-96, wherein the cell is a cancer cell.

98. A method of inducing ferroptosis in a cell, comprising contacting a cell with an effective amount of a compound, or a tautomer, stereoisomer, mixture of stereoisomers, isotopically enriched analog, or pharmaceutically acceptable salt thereof, of any one of claims 1-92.

99. The method of claim 98, wherein the cell is located within a subject.

100. The method of claim 99, wherein the subject is a human.

101. The method of any one of claims 98-100, wherein the cell is a cancer cell.

102. A method of treating a disease, disorder, or condition in a subject in need thereof, comprising administering to the subject a therapeutically effective amount of a compound, or a tautomer, stereoisomer, mixture of stereoisomers, isotopically enriched analog, or pharmaceutically acceptable salt thereof, of any one of claims 1-92.

103. The method of claim 102, wherein the disease, disorder, or condition is a cancer.

104. The method of claim 103, wherein the cancer is adrenocortical cancer, anal cancer, biliary cancer, bladder cancer, bone cancer, brain cancer, breast cancer, cervical cancer, colon cancer, endometrial cancer, esophageal cancer, head and neck cancer, intestinal cancer, liver cancer, lung cancer, oral cancer, ovarian cancer, pancreatic cancer, renal cancer, prostate cancer, salivary gland cancer, skin cancer, stomach cancer, testicular cancer, throat cancer, thyroid cancer, uterine cancer, vaginal cancer, sarcoma, or a soft tissue carcinoma.

105. The method of claim 104, wherein the cancer is osteosarcoma, glioma, astrocytoma, neuroblastoma, cancer of the small intestine, bronchial cancer, small cell lung cancer, non-small cell lung cancer, basal cell carcinoma, or melanoma.

106. The method of claim 103, wherein the cancer is a hematologic cancer.

107. The method of claim 106, wherein the hematologic cancer is acute lymphoblastic leukemia (ALL), acute myeloid leukemia (AML), lymphoma (e.g., Hodgkin's lymphoma, Non-Hodgkin's lymphoma, Burkitt's lymphoma), chronic lymphocytic leukemia (CLL), chronic myelogenous leukemia (CML), Hairy Cell chronic myelogenous leukemia (CML), or multiple myeloma.

108. The method of any one of claims 102-107, further comprising administering a therapeutically effective amount of additional therapeutic agent.

109. The method of claim 108, wherein the therapeutic agent is a platinating agent, an alkylating agent, an anti-cancer antibiotic, an antimetabolite, a topoisomerase I inhibitor, a topoisomerase II inhibitor, or an antimicrotubule agent.
